(19) United States
(12) Patent Application Publication
(10) Pub. No.: US 2025/0095770 A1
(43) Pub. Date: Mar. 20, 2025

(54) METHODS OF DESIGNING PRODUCTION OF MICROBIAL COMMUNITIES, METHODS OF PRODUCING MICROBIAL COMMUNITIES, AND MICROBIAL COMMUNITIES PRODUCED THEREBY

(71) Applicant: Wisconsin Alumni Research Foundation, Madison, WI (US)

(72) Inventors: Ophelia Venturelli, Madison, WI (US); Bryce Connors, Madison, WI (US); Brian Pfleger, Madison, WI (US)

(73) Assignee: Wisconsin Alumni Research Foundation, Madison, WI (US)

(21) Appl. No.: 18/729,316

(22) PCT Filed: Feb. 3, 2023
(86) PCT No.: PCT/US2023/012259

§ 371 Date: Jul. 16, 2024

Related U.S. Application Data
(60) Provisional application No. 63/306,691, filed on Feb. 4, 2022.

(51) Int. Cl. G16B 5/00; C12N 1/20; C12Q 1/02

(52) U.S. Cl. CPC G16B 5/00; C12N 1/20; C12Q 1/025

(57) ABSTRACT

The invention is directed to designing the production of microbial communities, methods of producing the microbial communities, and microbial communities produced thereby. The design of the microbial communities can include designing media compositions and/or inoculum ratios to control the makeup of consortia of microbes and thereby result in a defined microbial community composition. The designed media and/or inoculum ratios can be used to generate a microbial community having a defined composition of different microbes.

### FIELD OF THE INVENTION

The invention is directed to designing the production of microbial communities, methods of producing the microbial communities, and microbial communities produced thereby.

### BACKGROUND

Understanding and manipulating the microbiome has immense potential in human and animal health. Extensive research over the last decade has elucidated several beneficial associations between microbiomes and their human hosts. For example, communities of bacteria could be assembled to counter pathogenic invasion or subtractive technologies (e.g., phages) could be used to delete specific undesirable members of human-associated microbiomes.

Perhaps the most common example of manipulating human-associated microbiomes is through fecal matter transplants (FMT) for treating recurrent C. difficile colitis. While successful, FMT suffers from supply chain and safety issues, highlighting the need for improved processes for producing microbiomes at scale and for indications beyond colitis. As a result, several therapeutic companies are pursuing manufactured microbial communities comprising a defined and reproducible mixture of microorganisms. The current state of the art is to grow individual monocultures, which are then blended into a final product. While effective, this approach requires significant investments in equipment, skilled labor, consumables, documentation, and validation of many individual processes for FDA approval.

Single-vessel community culture poses an opportunity for cost and labor reduction. Methods for designing the production of microbial communities in, for example, single-vessel community culture and methods of producing such microbial communities are needed.

### SUMMARY OF THE INVENTION

The invention is directed, in part, to designing media and/or inoculum ratios to control the makeup of consortia of microbes and thereby result in a defined microbial community composition. The invention is also directed, in part, to employing the designed media and/or inoculum ratios to generate a microbial community having a defined composition of different microbes. The invention is also directed, in part to microbial communities produced with the designed media and/or inoculum ratios.

One aspect of the invention is directed to methods of designing production of a microbial community comprising different microbe subsets of a set of microbes in a proportion approximating a target proportion. The methods can comprise designing a target medium and/or designing a target-inoculum-density profile of the microbe subsets.

Designing the target medium can comprise growing each of the microbe subsets separately to carrying capacity in each of multiple test media comprising different test-concentration profiles, determining proportion of microbe subset carrying capacities in each test medium to thereby obtain test-medium proportions corresponding to the test-concentration profiles, and determining from the test-medium proportions and the corresponding test-concentration profiles a target-concentration profile yielding or predicting to yield a target-medium proportion of microbe subset carrying capacities that differs from at least one of the test-medium proportions. The target medium can then be defined as comprising the target-concentration profile.

Designing the target-inoculum-density profile can comprise growing to stationary phase the microbe subsets together in each of multiple inoculum test cultures inoculated with different test-inoculum-density profiles of the microbe subsets, determining proportion of the microbe subsets at stationary phase in each inoculum test culture to thereby obtain test-inoculum-density proportions corresponding to the test-inoculum-density profiles, and determining the target-inoculum-density profile from the test-inoculum-density proportions and the corresponding test-inoculum-density profiles. The target-inoculum-density profile preferably yields or is predicted to yield a target-inoculum-density proportion of the microbe subsets that differs from at least one of the test-inoculum-density proportions.

In some versions, the methods can further comprise designing initial-test-inoculum-density profiles for testing in the design of the target-inoculum-density profile as outlined above. Designing the initial-test-inoculum-density profile can comprise growing each of the microbe subsets separately in a set of first inoculum test cultures and measuring kinetic growth of each microbe subset in its first inoculum test culture, growing each of the microbe subsets together in a second inoculum test culture and measuring total growth limit of the microbe subsets in the second inoculum test culture, modeling the kinetic growth of each microbe subset and the total growth limit to solve for a reference-inoculum-density profile, and determining the initial-test-inoculum-density profiles as points encompassing the reference-inoculum-density profile in a multivariate design space.

Another aspect of the invention is directed to methods of producing a microbial community. The methods can comprise inoculating a microbial community medium with the microbe subsets and growing the microbe subsets. In some versions, the microbial community medium is the target medium. In some versions, the microbial community medium is inoculated with the microbe subsets in the target-inoculum-density profile. In some versions, the microbial community medium is the target medium and is inoculated with the microbe subsets in the target-inoculum-density profile.

Another aspect of the invention is directed to microbial communities produced by any method described herein.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1E. High-throughput monoculture workflow for improving community diversity. FIG. 1A. (a) Sugar mixture (glucose, arabinose, and maltose), yeast extract (Y.E.), defined amino acid mixture (Aminos), and pH are varied in a common base medium (methods) according to a half-factorial experimental design (1-8) with a center-point (9). Grayscale shading indicates scaled design levels (black indicates high, white indicates low), with corresponding concentrations labeled on the heatmap in units of g/L or pH. (b) Monoculture growth response of the ten species in each of nine media conditions is shown as the carrying capacities of logistic differential equation fits to timeseries data (equation 3, FIGS. 2A-2G). Species are ordered alphabetically left to right, with labels and shading per panel e. Carrying capacities (bars indicate mean, diamonds indicate replicates, n=3 to 4) plotted on the y-axis. (c) “Monoculture-diversity” of the mean of carrying capacities of the 10 species on each medium is shown as gray horizontal bars. Diamonds indicate monoculture-diversities calculated from like replicates. Dashed line indicates maximum possible monoculture-diversity for ten species. (d) Computationally optimized medium recipe. An optimization algorithm calls the regression models (FIG. 1B) to solve for the unique set of media component concentrations that maximizes the monoculture-diversity of predicted carrying capacities (equation 7). (e) Measured carrying capacities of each species on the optimized medium (d). FIG. 1B. A linear regression model is fit to predict each species' carrying capacity (b) from the media component setpoints (a). Bipartite network visualization of linear regression model parameters, which maps growth effects to media components (equation 6). Species are shown as nodes in the left column, media component predictors shown as nodes in the right column. Edge thicknesses indicate scaled median parameter values (methods). For visual simplification, parameters with a value less than 0.05 are not shown, and quadratic main effects are summed with first-order main effects. FIG. 1C. Scatter plot of measured vs. predicted monoculture-diversity. Predicted monoculture-diversity is calculated from validation/test predictions (of carrying capacities), which are comprised of out-of-fold predictions plus the new, optimized condition. FIG. 1D. Benchmarking endpoint community compositions, cultured from an even inoculum proportion in the baseline medium (condition 7), the highest monoculture-diversity screened medium (4), and the optimized medium (10). Species indicated by the partitions in the stacked bars (from bottom to top) are Blautia hydrogenotrophica (BH), Bacteroides unformis (BU), Dorea longicatena (DL), Eubacterium rectale (ER), Prevotella copri (PC), and Parabacteroides johnsonii (PJ) in the baseline medium (7); BU, Collinsella aerofaciens (CA), DL, ER, PC, and PJ in the highest monoculture-diversity screened medium (4); and BH, BU, CA, DL, Egerthella lenta (EL), ER, PC, and PJ in the optimized medium (10). Shannon diversity of average community composition (n=3 replicates, equation 1) is indicated as gray solid bars, righthand axis. Shannon diversities calculated from like sets of replicates are overlaid as diamonds. FIG. 1E. pH change in best screened vs. optimized medium, as resulting from the lowered sugar concentration. Both fermentations start at an initial pH of 5.7 (bar plot baseline). The final pH is indicated by the bar height.

FIGS. 2A-2G. Timeseries measurements and logistic fits for media screening experiment and optimized medium. FIGS. 2A-2E. Media component screening experiment (corresponding to FIGS. 1A-1E). Timeseries growth curves for each species on each media, n=4 replicates (circles) of experimental data are each fit with a logistic differential equation parameter set. Shading corresponds to the media condition as indicated in the heatmaps in the Media Legend and Scaled Design Matrix. The absolute concentrations of the components are labeled on the heatmap in FIG. 1A (panel a). FIGS. 2F and 2G. Growth curves and logistic fits on optimized medium. All cultures are inoculated at 0.01 OD600.

FIGS. 3A-3C. Media regression model parameters referenced in text for BH (FIG. 3A), BL (FIG. 3B), and DL (FIG. 3C). Boxplots indicate distribution of parameter value across leave-one-out parameter sets. Bar heigh indicates median value across leave-one-out parameter sets. Parameters with median values larger than 0.05 have the corresponding predictor labeled on the x-axis. The value on the y-axis indicates the amount of growth attributed to the corresponding predictor. For example, the BL model effectively includes only a sugar parameter. Thus, the expected growth is ŷ=/β_{sugar}*X_{sugar}, where ŷ is growth (OD), β_{sugar} is the plotted regression (OD per grams per liter), and X_{sugar} is the concentration of sugar in grams per liter.

FIGS. 4A-4C. Regression model predictions and statistics. For each species, a linear regression model is used to predict the carrying capacity parameters for the logistic fits shown in FIGS. 2F and 2G as a function of the four media component variables shown in the heatmap of FIGS. 2A-2E. FIG. 4A. Nested cross validation is used to fit nine “leave-one-out” parameter sets. The out-of-fold predictions are shown in the left panel and predictions on training data are shown on right panel. Goodness of fit statistics (Pearson rho, p) are calculated between the measured data and the average prediction across leave-one-out parameter sets, such that the number of predictions is equal to the number of datapoints and statistics are not biases by multiple predictions per experimental datapoint. FIG. 4B. Species-specific Pearson correlation of out-of-fold model predictions vs. measured. FIG. 4C. Validation/test predictions for monoculture-diversity (left panel), and training data (right panel). Goodness of fit is calculated between the average of the training data predictions across parameter sets (8 points of a similar shading) and the measured value. The “out-of-fold monoculture-diversity” is calculated with equation 2.

FIG. 5. Graphical illustration of cross-validation partitioning for a sparse, small, experimental design dataset vs. randomly sampled large dataset (a more typical data-science type approach). (a) Out-of-fold predictions for sparse experimental designs force the model to “extrapolate” into a design space that they have never been trained on, whereas (b) randomly sampled datasets are probably able to “interpolate” between training datapoints to a high degree. For this reason, we show both in-fold and out-of-fold predictions in FIGS. 4A-4C to build intuition about the models' predictivities and utility, and why this type of validation is not commonly done in the DoE literature. When the models are used for optimization, they are never forced to “extrapolate,” since the design samples the extrema of the design space, and all possible new conditions are constrained within this design space.

FIGS. 6A-6F. Forecasting community dynamics from monoculture kinetics and a total population constraint. FIG. 6A. A model is derived that allows prediction of major trends in community assembly using only monoculture kinetic data and an empirical total community growth threshold. The model comprises logistic equations that are coupled via a total growth constraint term. We refer to the model as a “constrained system of logistic equations” or “CSLE” (equation 9 and Table 1). FIG. 6B. Monoculture kinetic data (OD600, filled circles) over a wide range of inoculum densities (0.01 to 1e−7 OD600 by 10-fold serial dilution). Inoculum densities that did not yield reproducible growth are omitted (FIGS. 7A-7C). A single logistic differential equation parameter set (μᵢ, Kᵢ) is fit to each species' set of growth curves (solid lines, Methods). FIG. 6C. Endpoint total growth of a 10-member community culture (of unknown species composition) is six-fold lower than the sum of the 10 independent monocultures, indicating substantial negative interactions between species. This observation motivates the total population constraint term in the CSLE model. The value of the constraint parameter K_{comm} (“community carrying capacity”) is set as the measured optical density of the full 10 species community culture. FIG. 6D. Comparison of CSLE model predictions, (y-axis, left-hand stacked bar) with experimentally measured community relative abundance data (x-axis, right-hand stacked bar). Pearson correlation coefficient and p-value are indicated by “rho” and “p”, respectively. FIG. 6E. In comparison, modeling community assembly as the sum of independent logistic equations (y-axis, right-hand stacked bar) fails to predict community composition (relative abundance, x-axis, left-hand stacked bar). Species indicated by the partitions in the left-hand stacked bar (from bottom to top) are BH, BL, BU, CA, DL, EL, ER, FP, PC, and PJ. Species indicated by the partitions in the right-hand stacked bar (from bottom to top) are BH, BU, CA, DL, EL, ER, PC, and PJ. FIG. 6F. The steady state composition of the CSLE model is a function of initial conditions. We use optimization to solve for initial conditions (e.g. inoculum) that maximizes endpoint Shannon diversity (equation 10). This set of initial conditions is later used as a reference point to guide community experimental design (FIGS. 9A-9H).

FIGS. 7A-7C. Monoculture growth kinetics from high to low inoculum densities.

Inoculum levels from standard (0.01 OD600) to very low (10E−7 OD600) levels are evaluated by performing 10-fold vol/vol serial dilutions of the highest inoculated condition for each species. A logistic differential equation:

$$\frac{dx}{dt} = μ ( 1 - \frac{x}{K} ) x$$

with standard species specific parameter set of growth rate and carrying capacity (mu,K) is fit to each species' set of monoculture exponential phase growth data (yellow to purple indicate increasingly low dilution levels). As visualized on the semilog axes, the majority of cell doublings are predicted to occur below the limit of detection of the platereader (dotted horizontal line) for lower inoculum levels. The “true” inoculum cannot be directly measured at these levels, it is calculated from a measurable value in the linear range of the platereader divided by the serial dilution factor. To circumvent these measurement limitations, we fit the initial conditions of the ODE (X_{0,i}, or inoculum) as a parameter in our nonlinear regression (methods). The fitted initial condition parameters are mapped to the calculated initial conditions according to dilutions performed experimentally, and the inoculum levels that fall on a linear trendline in log-log space are displayed in column three (and used in subsequent inoculum optimization). This mapping accounts for density dependent growth effects, lag phase, and exponential growth below LOD.

FIGS. 8A and 8B. Motivating the fitting of initial conditions for the low inoculum density experiment. Sample goodness of fit plots of model vs. data for fitting a logistic differential equation to several species' sets of growth curves from inoculum densities of 0.01 (left-most curve, except in BL, in which the left-most curve was 1e−6) through 1e−7 (right-most curve). FIG. 8A shows model fits using known (target) inoculum density for initial conditions, while in FIG. 8B the initial conditions were omitted from training data and fitted as unknown parameters. This was performed because the serial dilutions of the 0.01 OD inoculum density were well below the LOD of a plate reader (approximately 0.001-0.01). As such, the majority of exponential cell-doubling occurred well below the limit of detection of the plate reader for lower inoculum densities, causing the readouts to appear to be in an extended “lag phase”, which was actually just a constant readout of the LOD of the plate reader. The fitting of initial conditions captures exponential growth below plate reader LOD, lag phase, and growth which may occur during experimental setup prior to collecting the time zero reading. However, given what we learned throughout the study, we suggest that that the accuracy improvement of this procedure may not merit the complexity, and would suggest just fitting the constrained system of logistic equations to a training dataset comprising all species' sets of exponential phase data, stationary phase data, and known initial conditions (e.g. omit all data below 0.05 OD600, omit death phase data, and append target inoculum densities as initial conditions). The suggested parameter fitting procedure would then be similar to how the gLV model is fit to all monoculture+community data, although in the CSLE case there would be no community data.

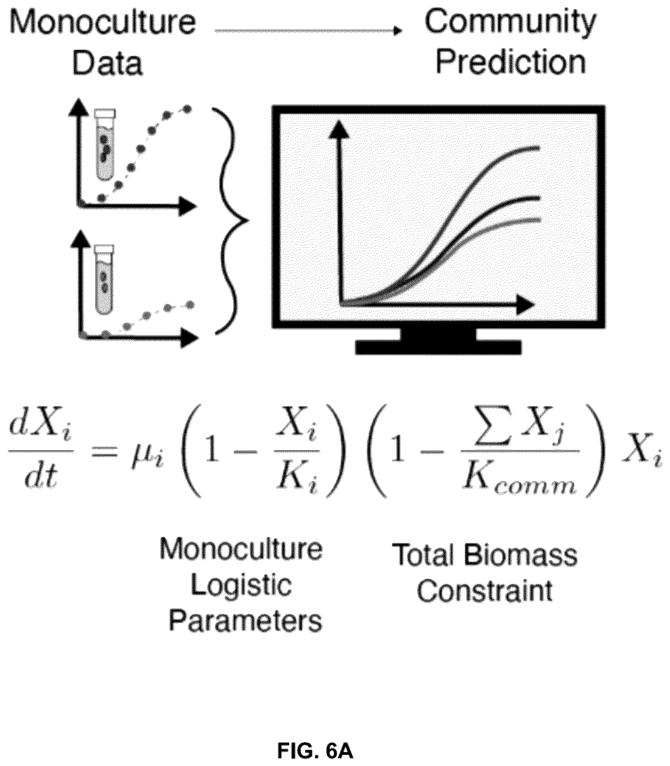

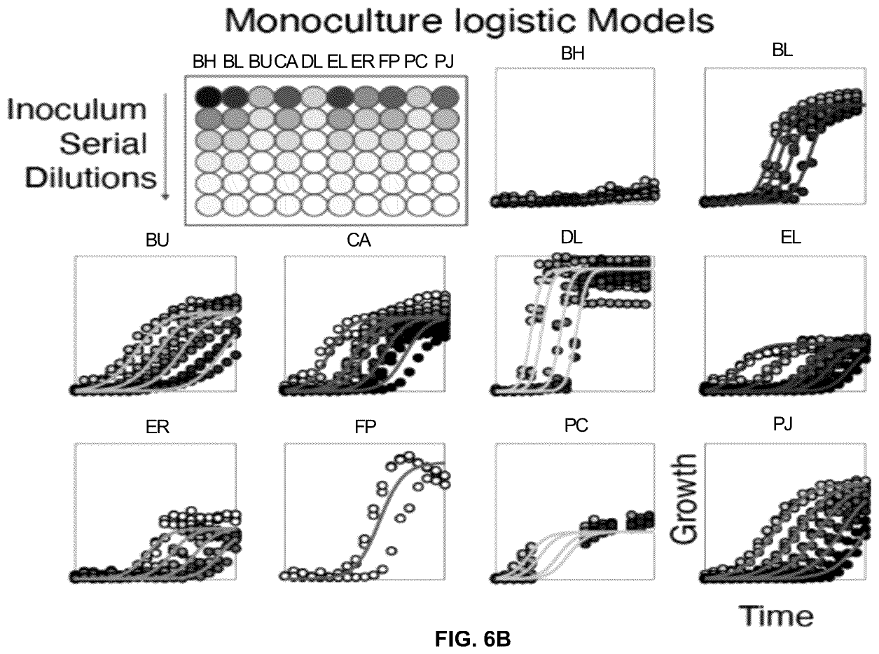

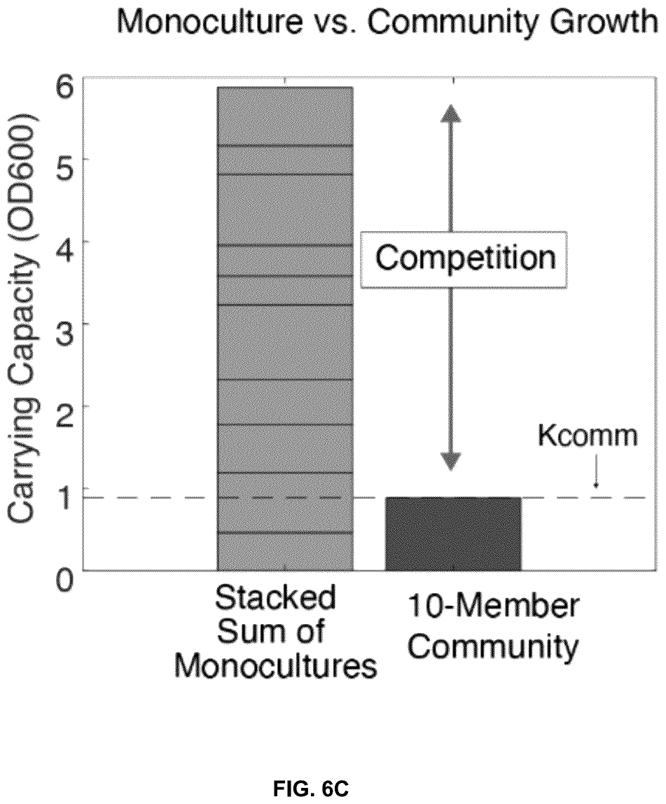

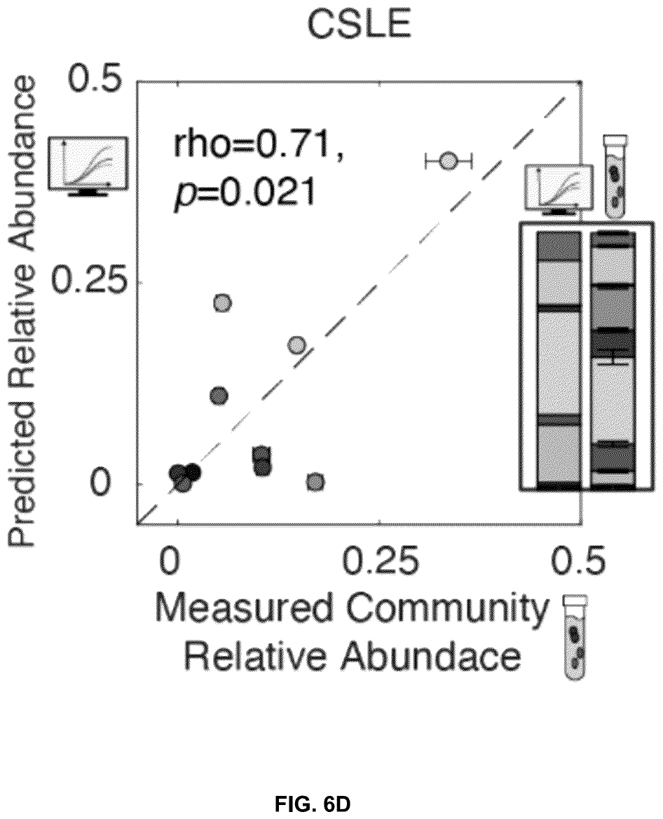

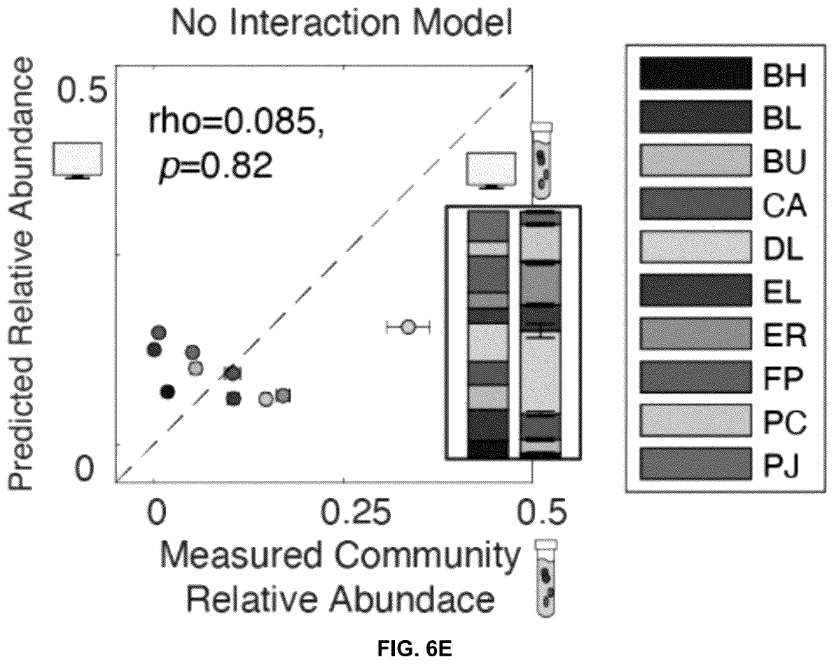

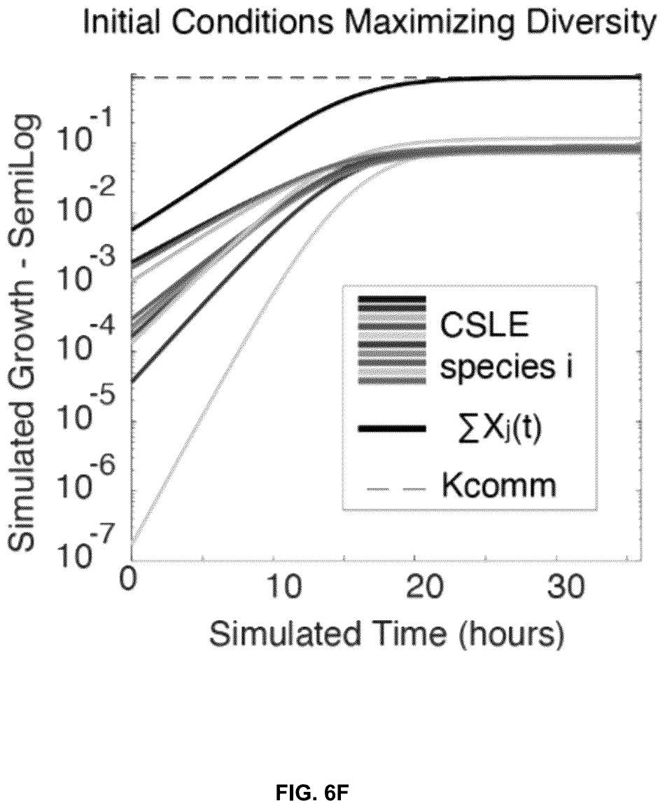

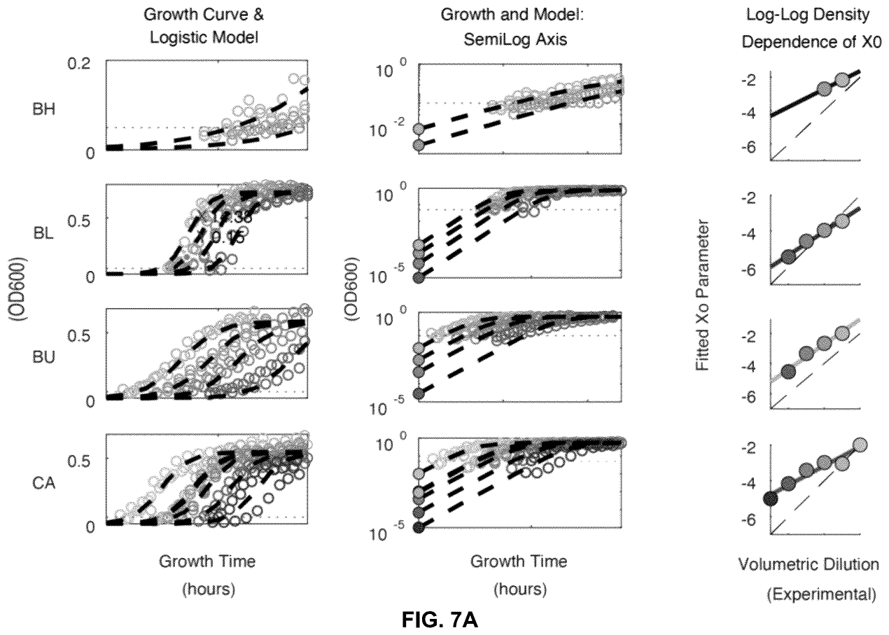

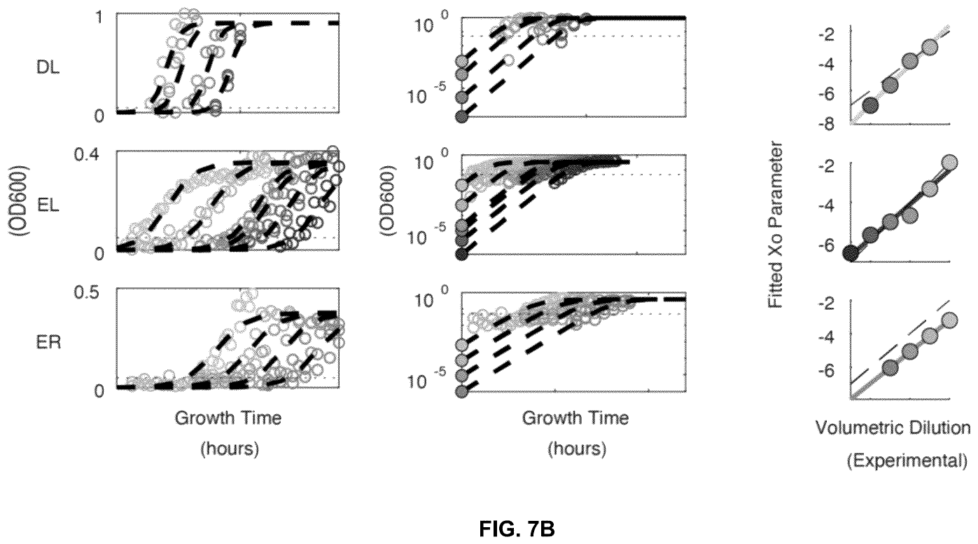

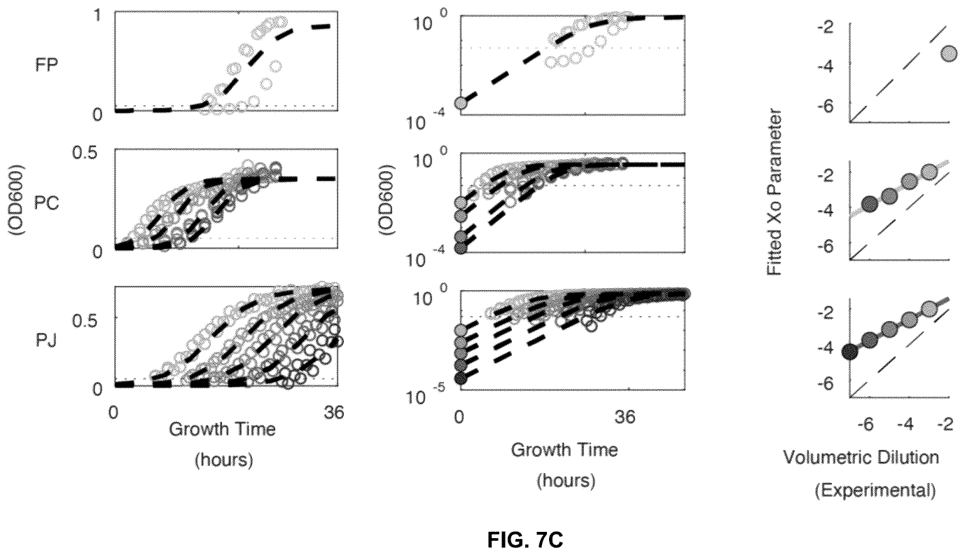

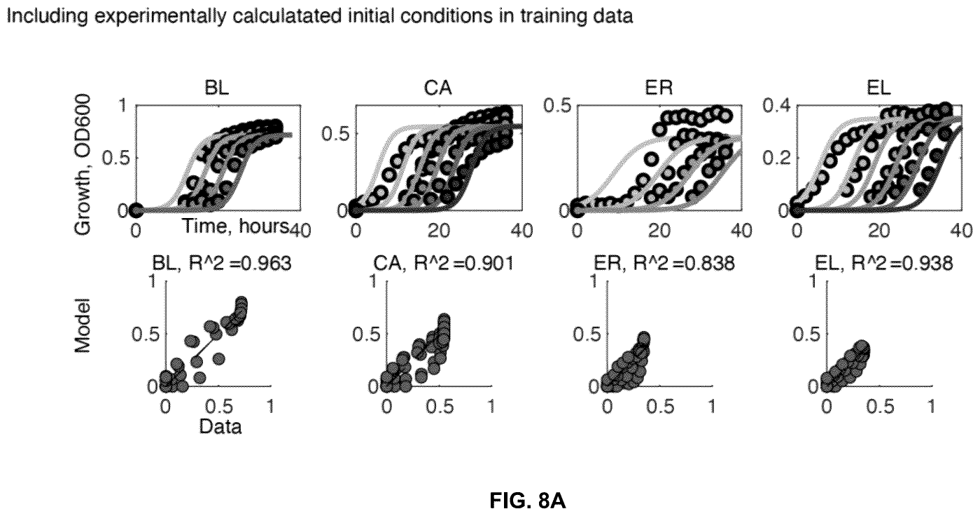

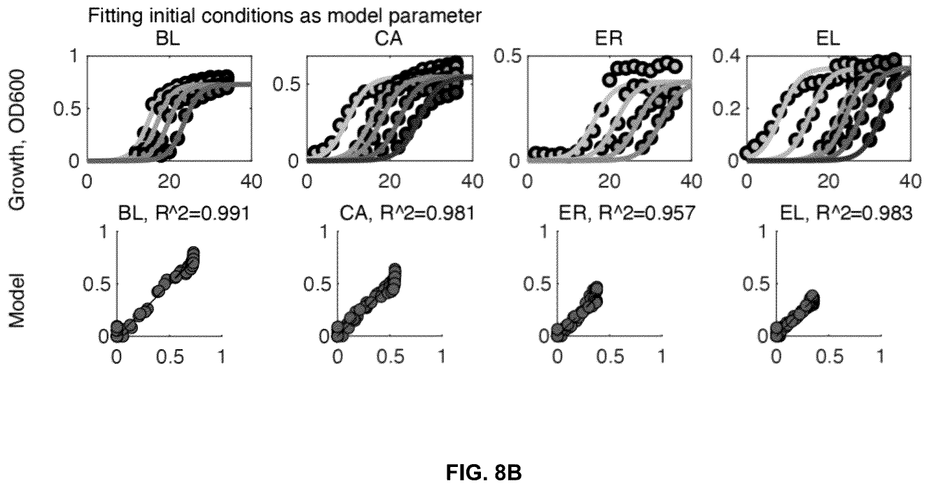

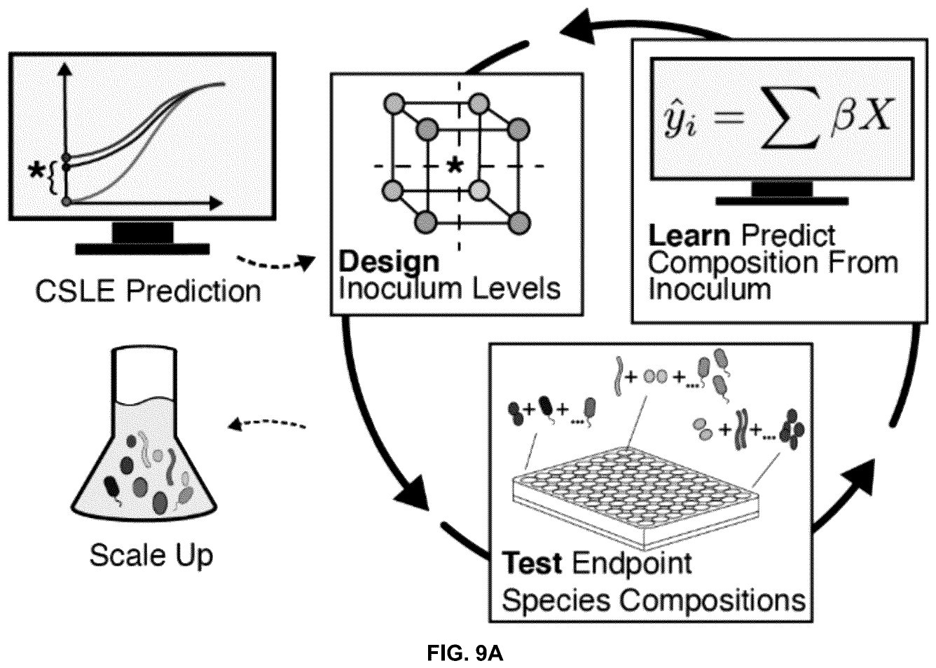

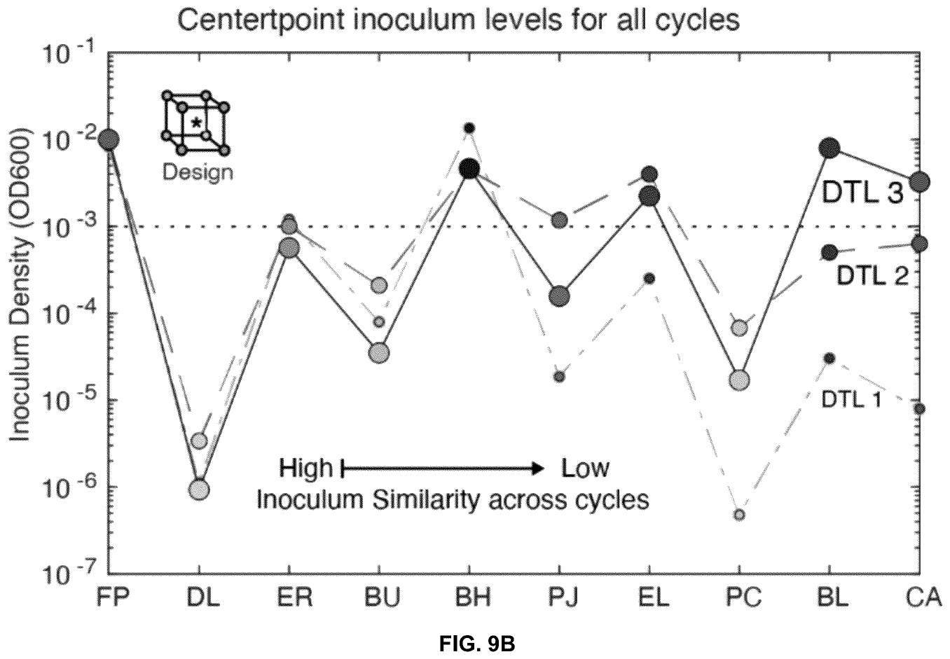

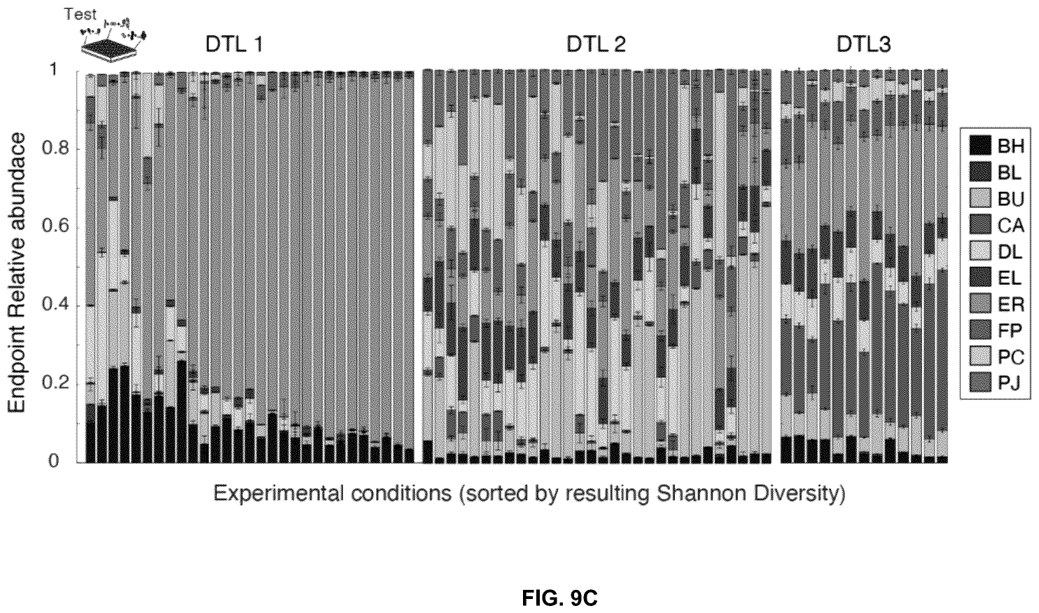

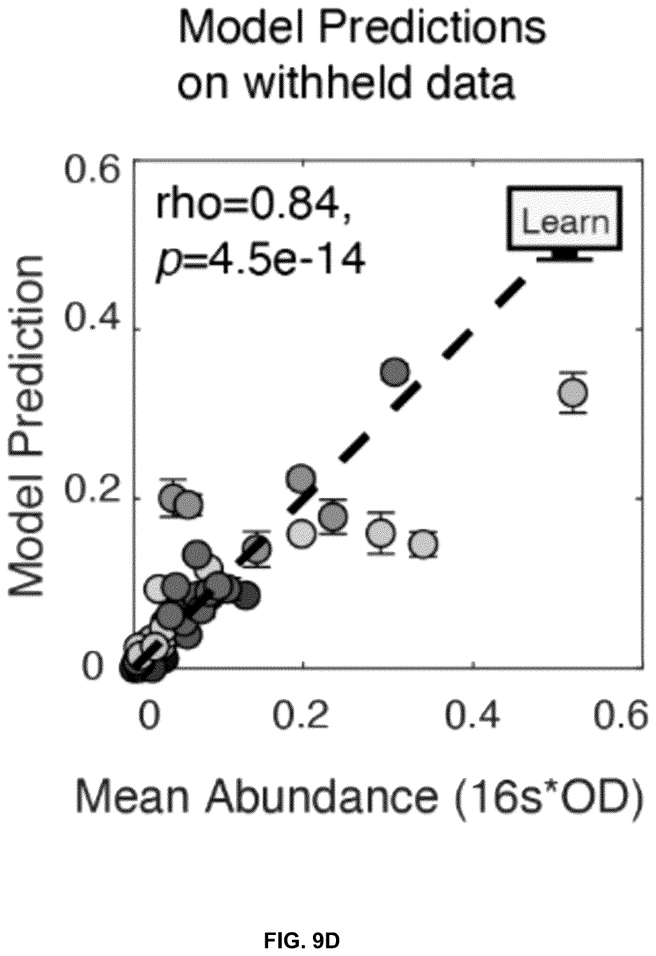

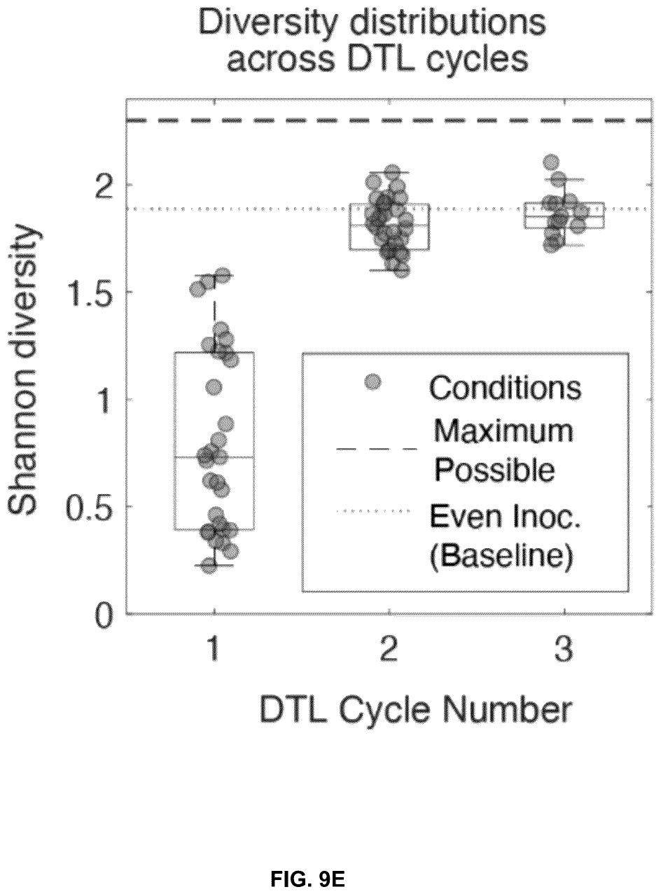

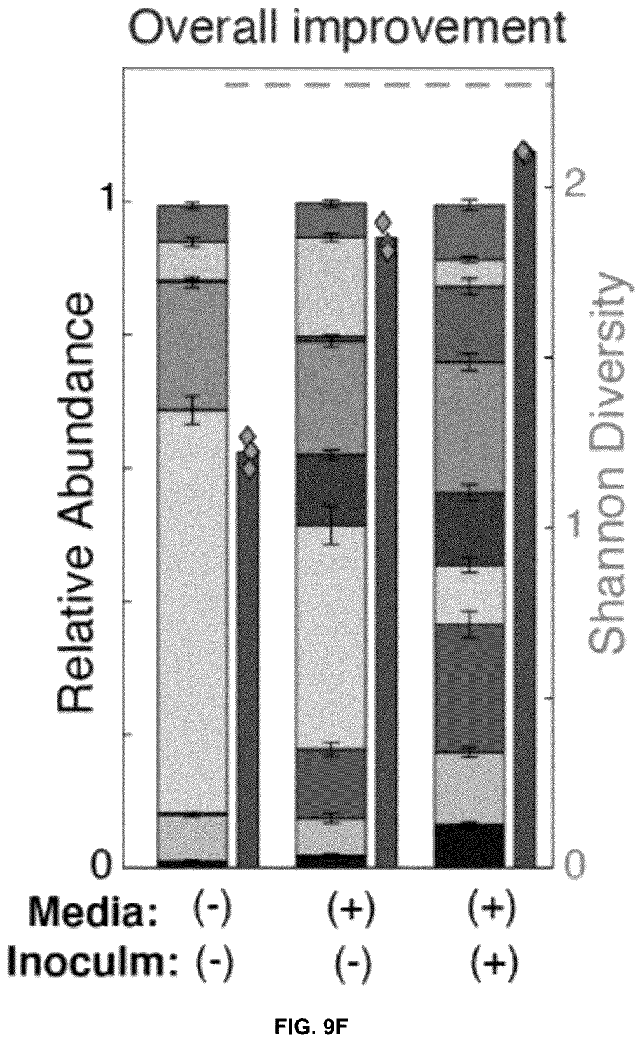

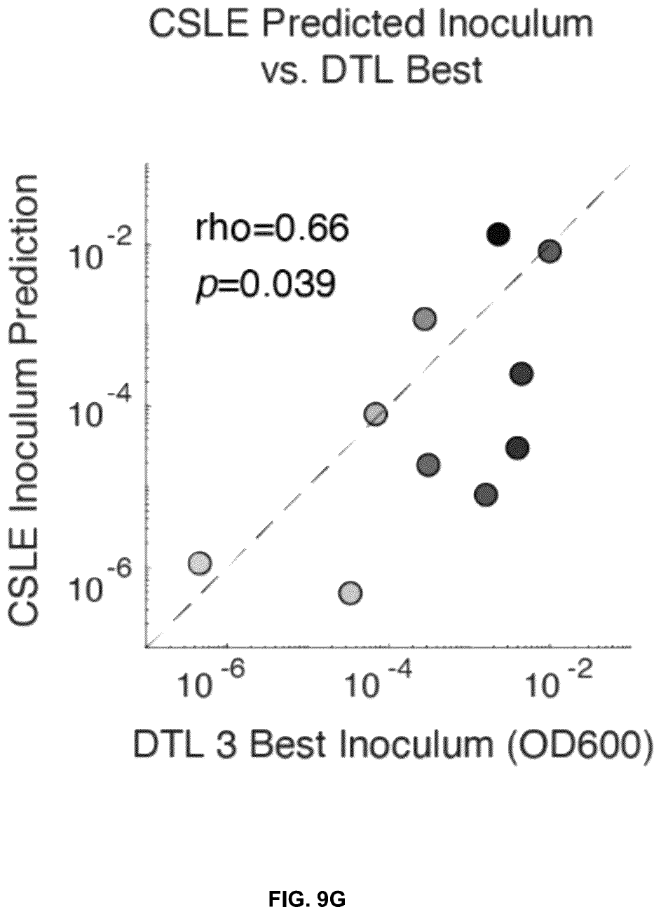

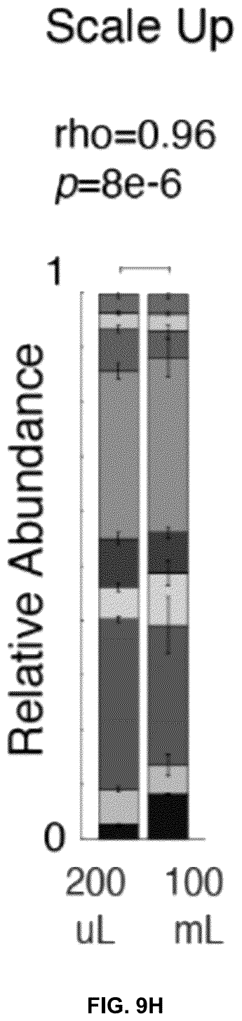

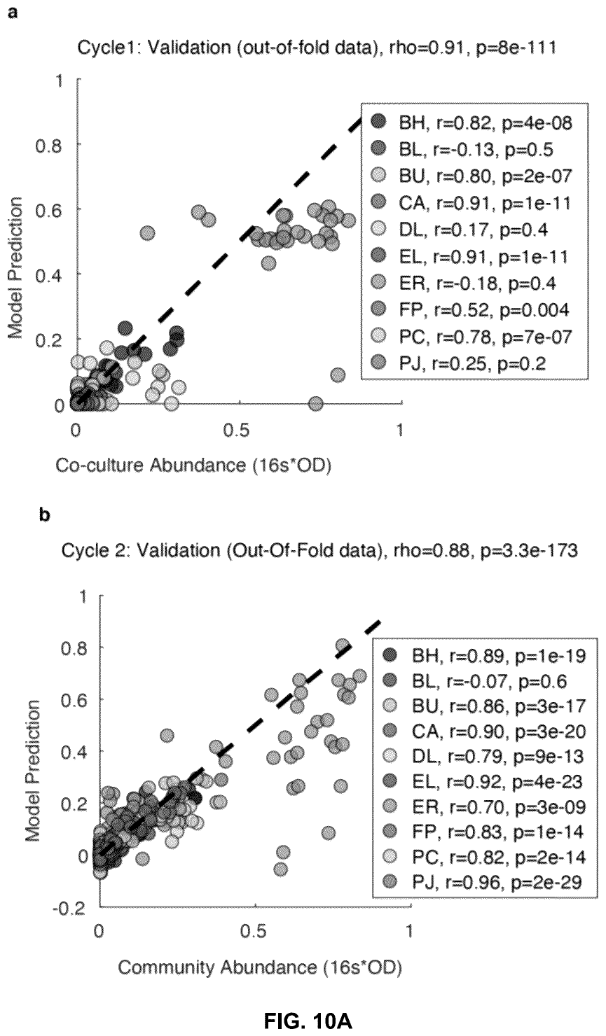

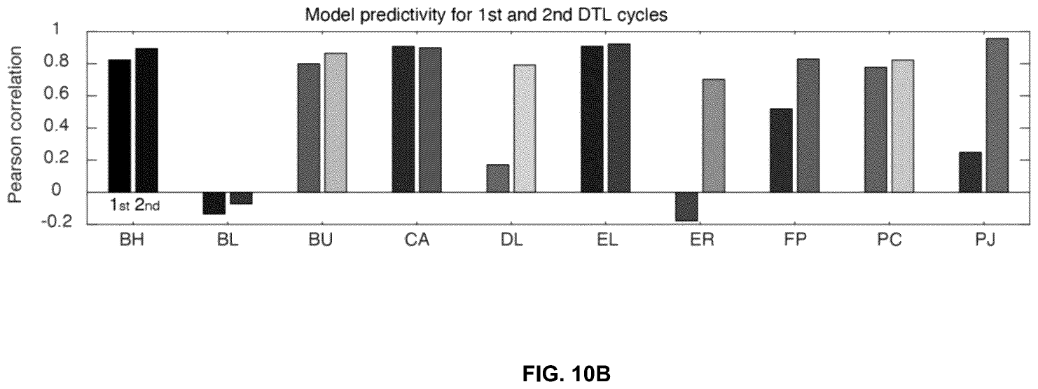

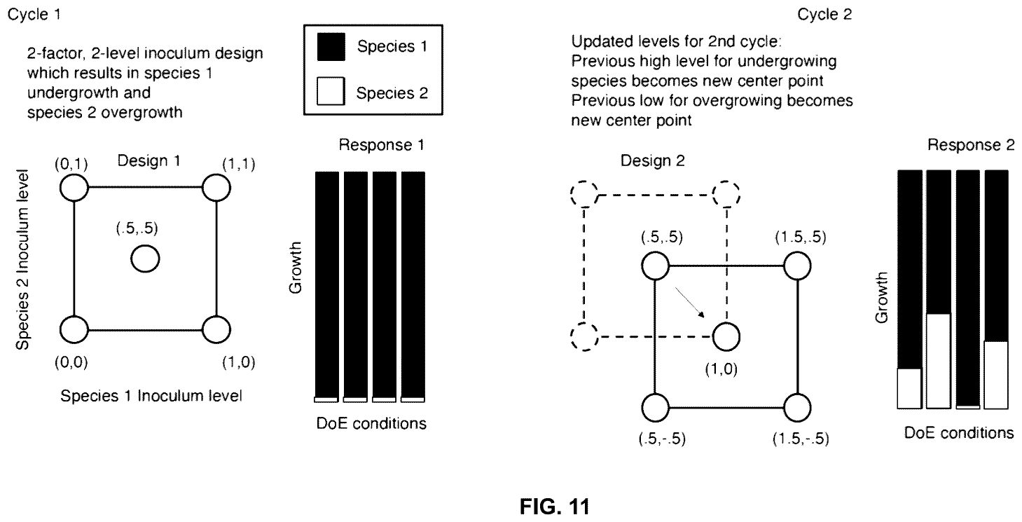

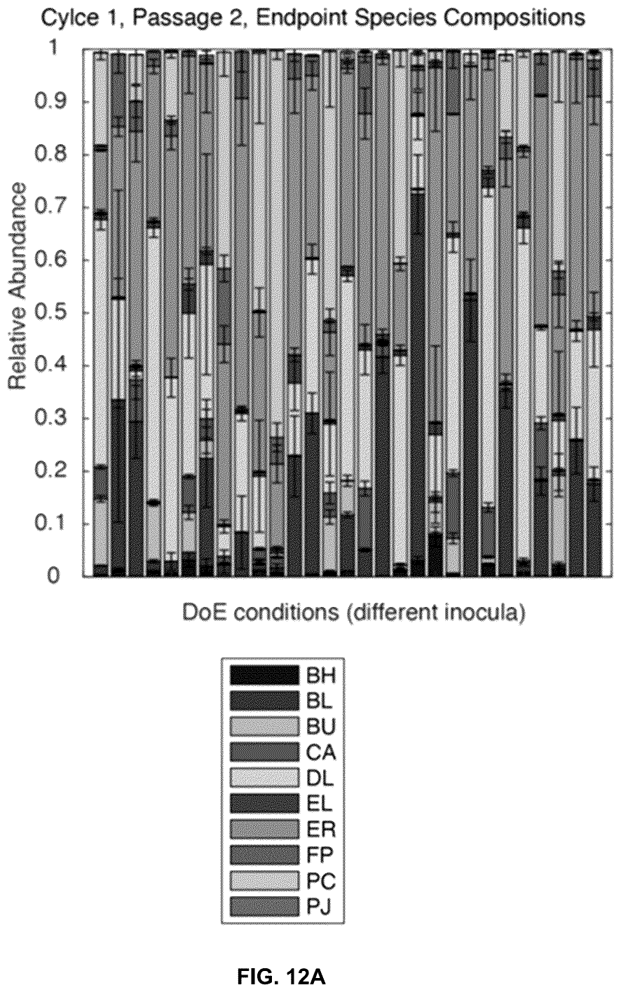

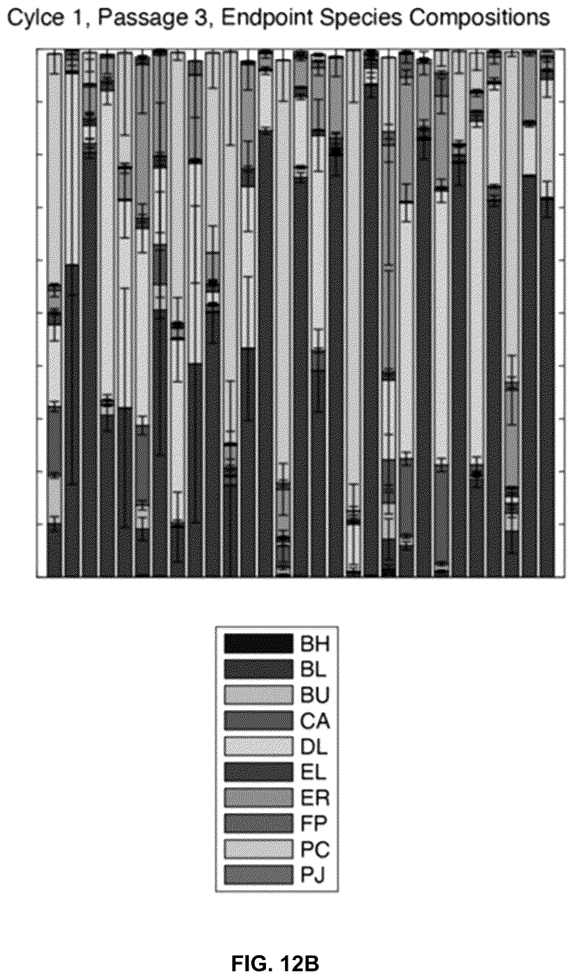

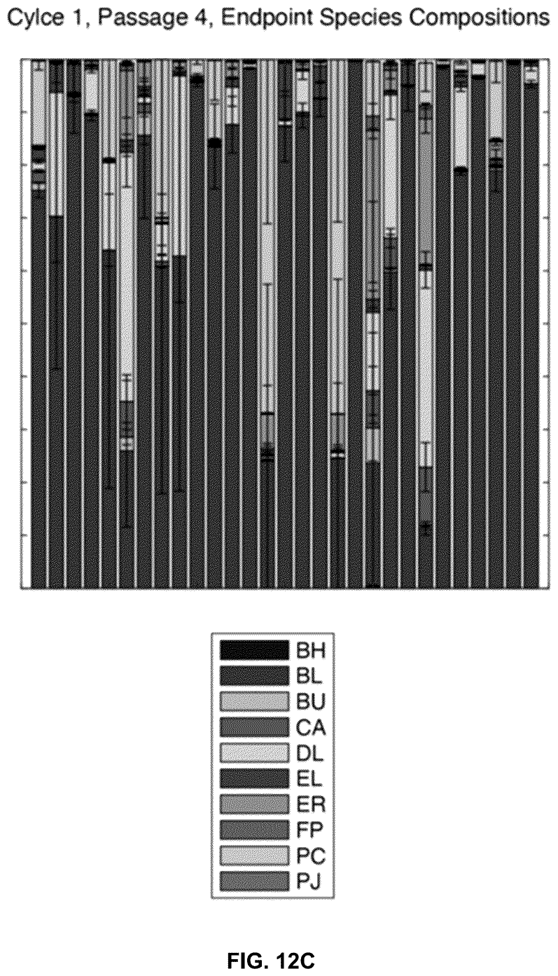

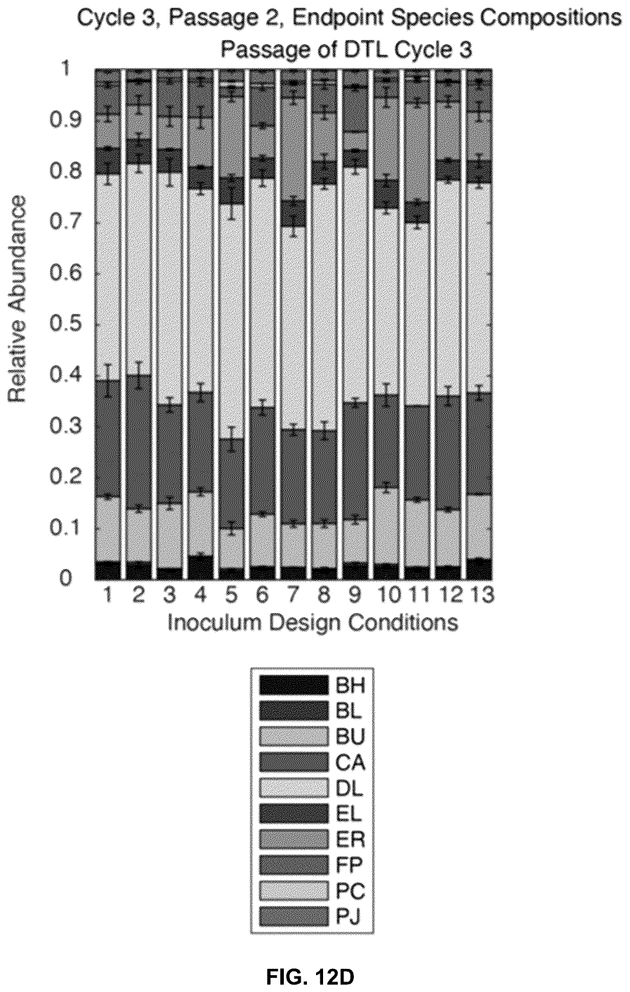

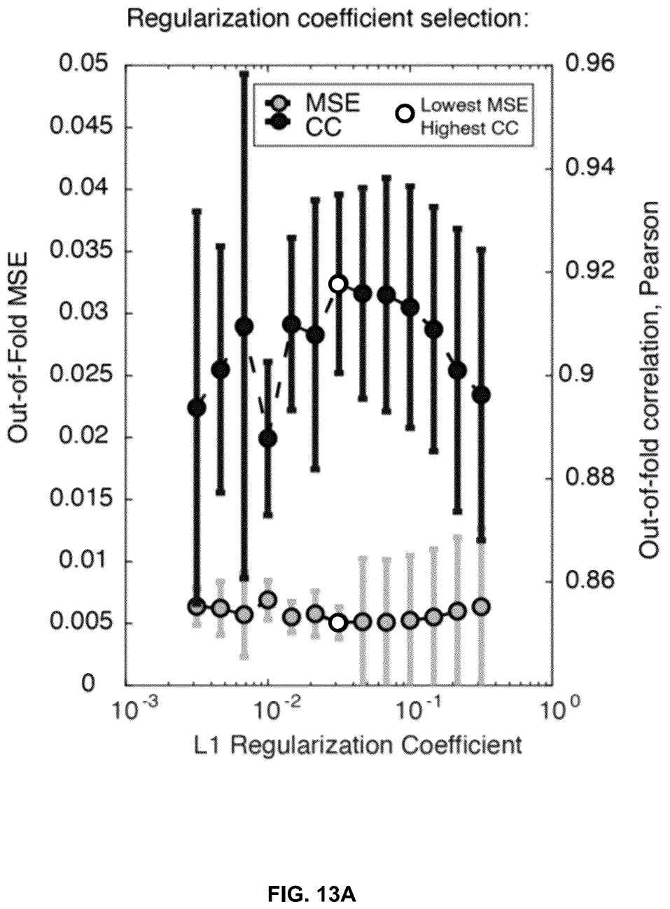

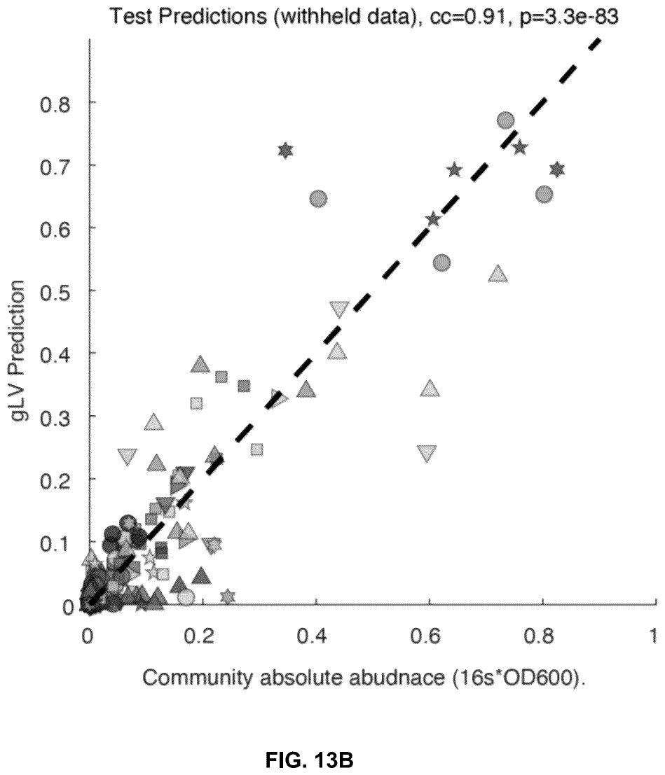

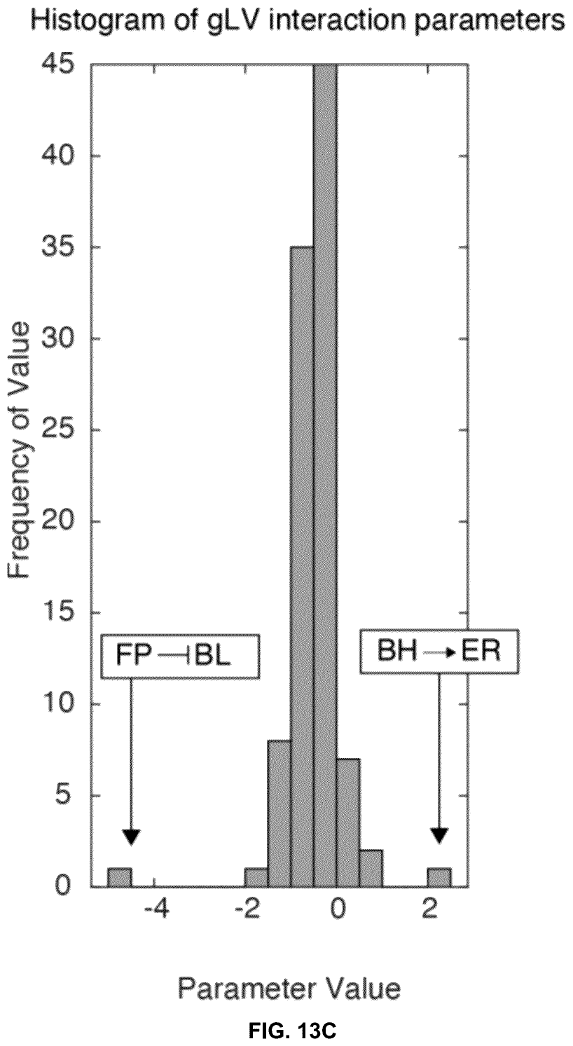

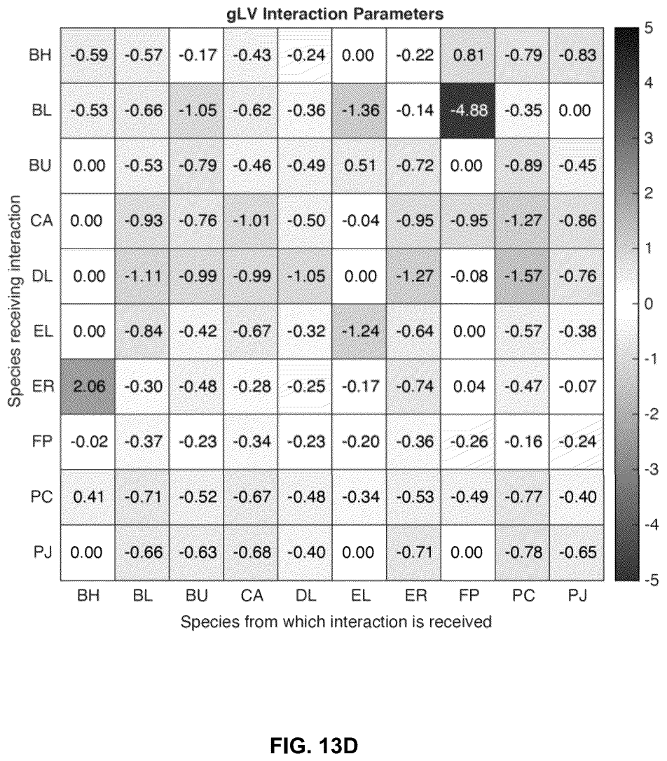

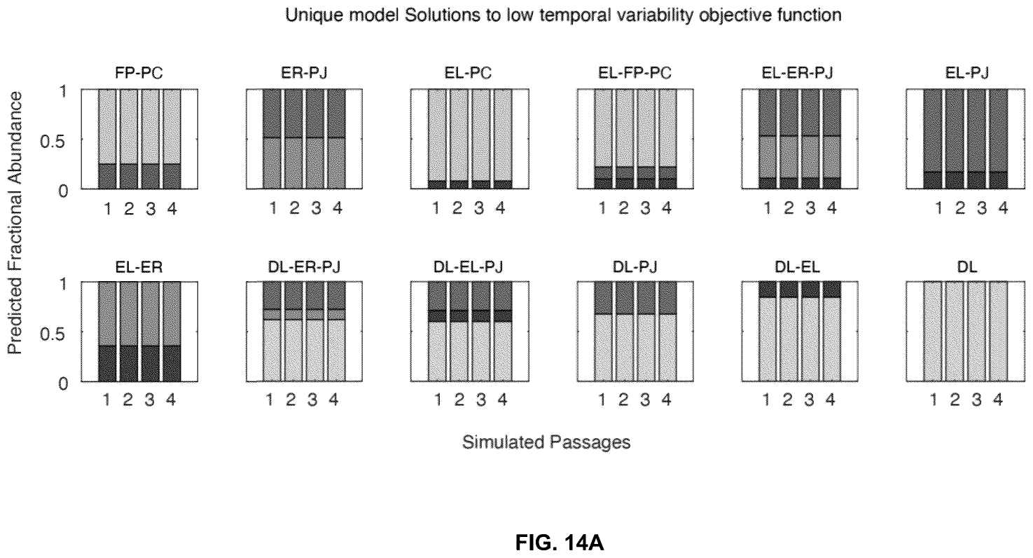

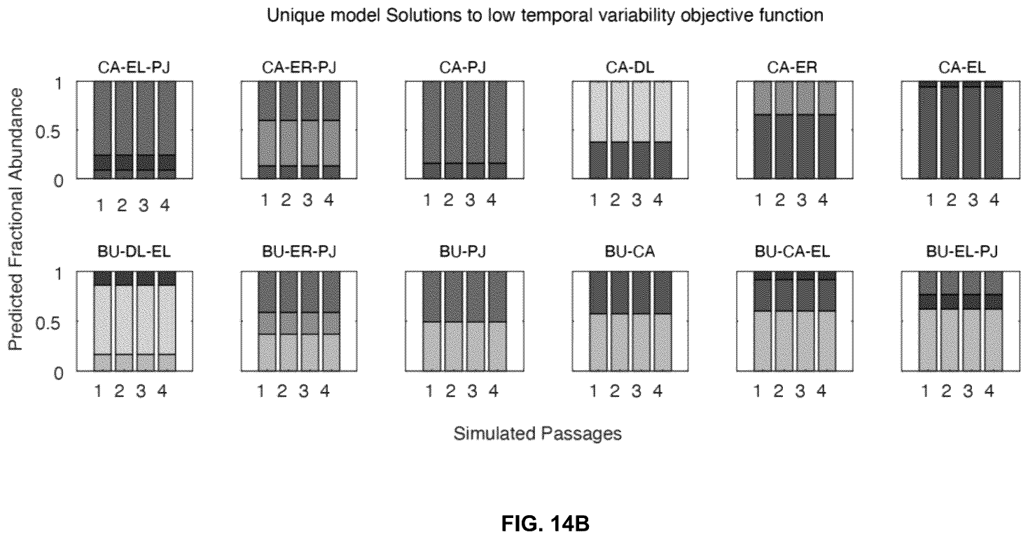

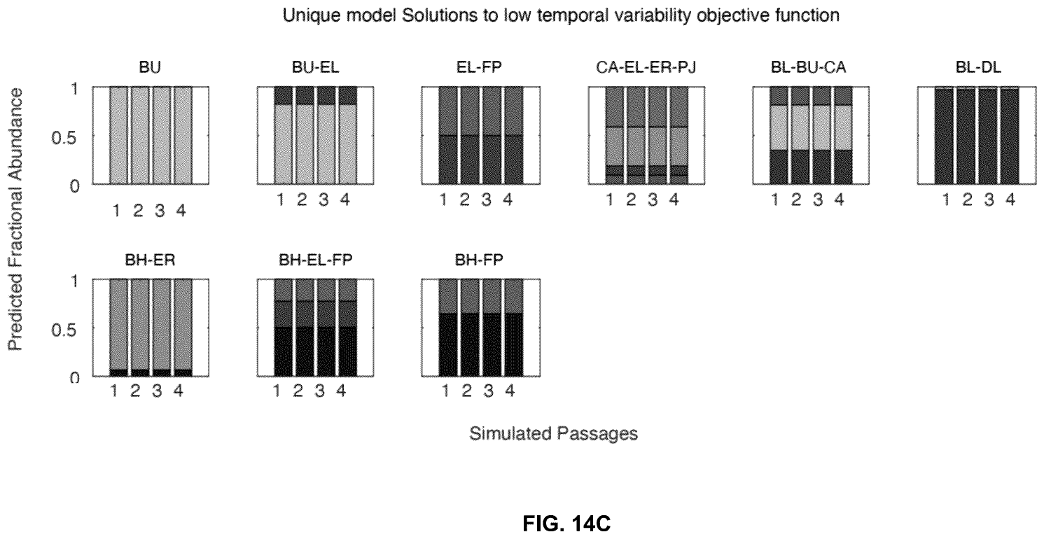

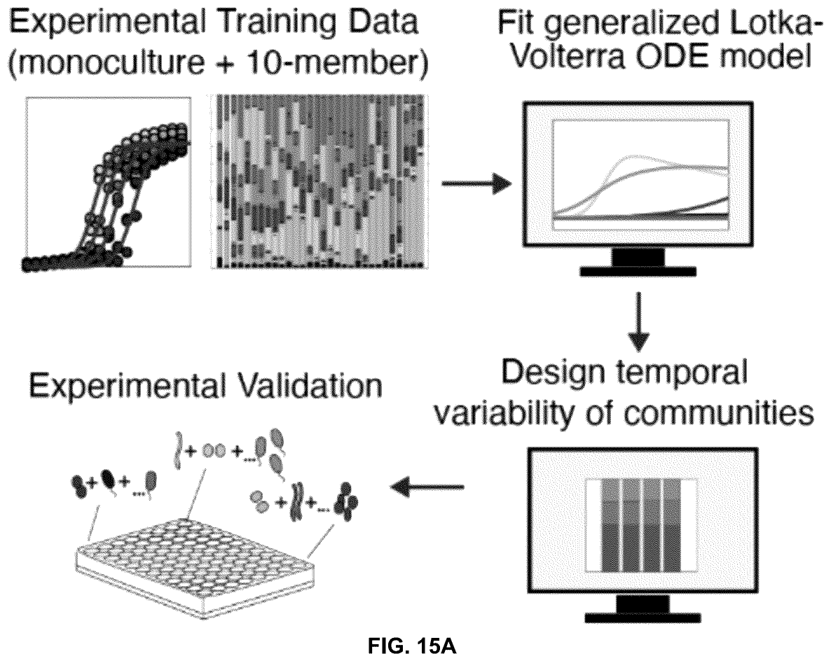

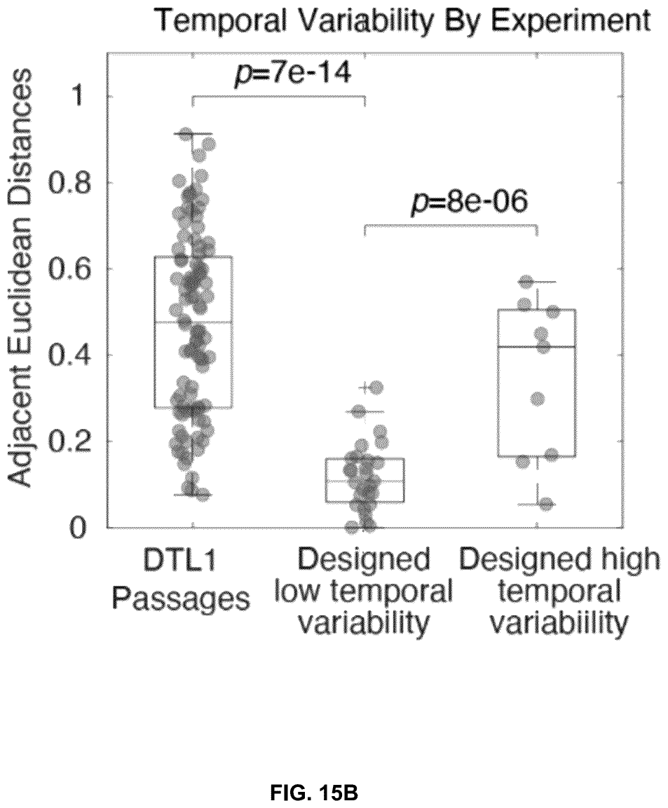

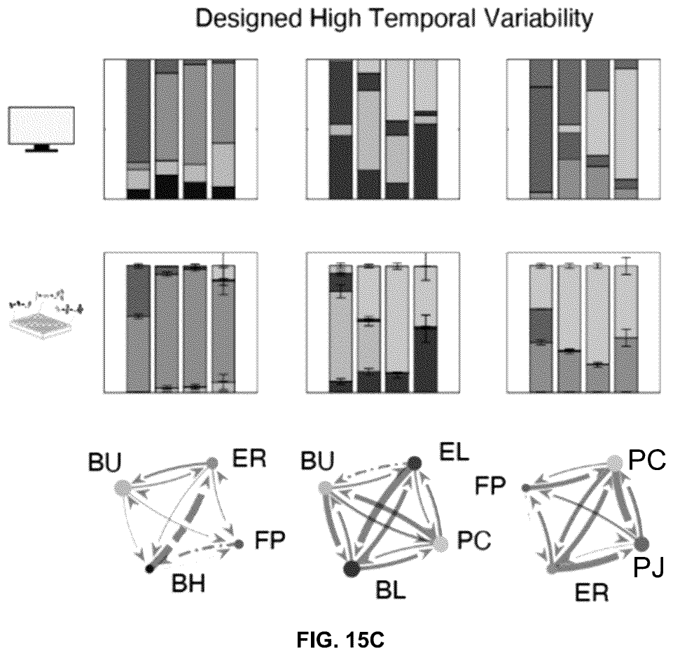

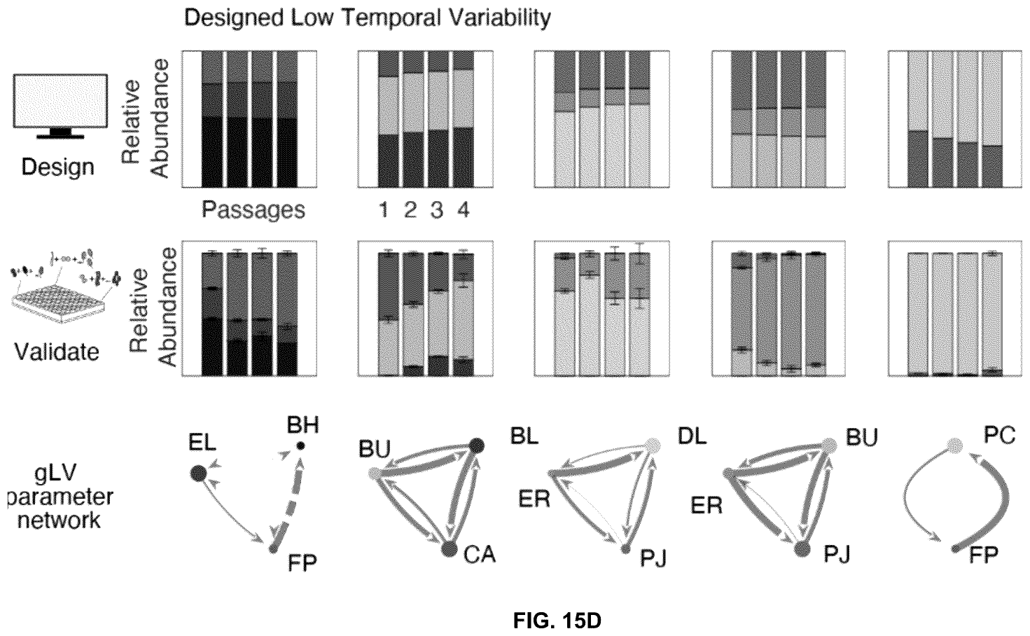

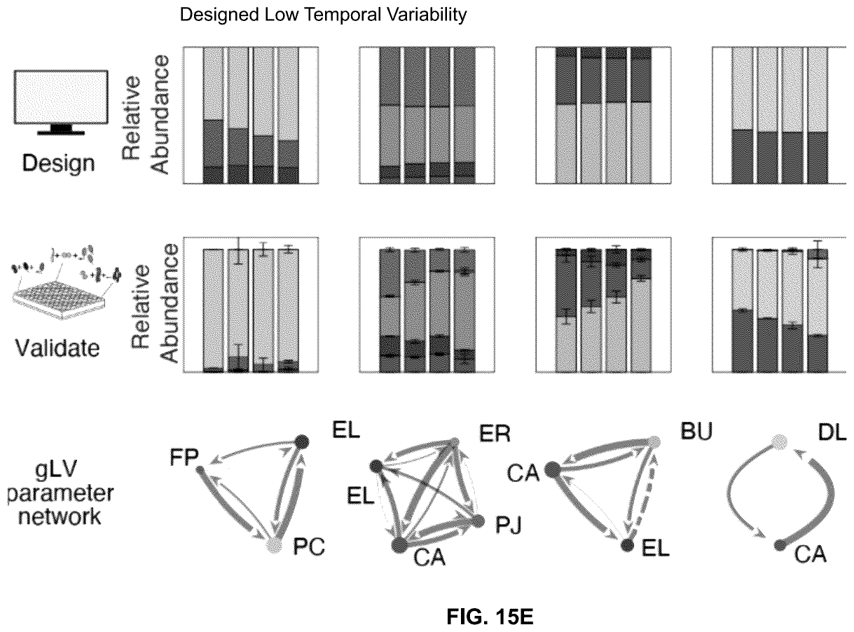

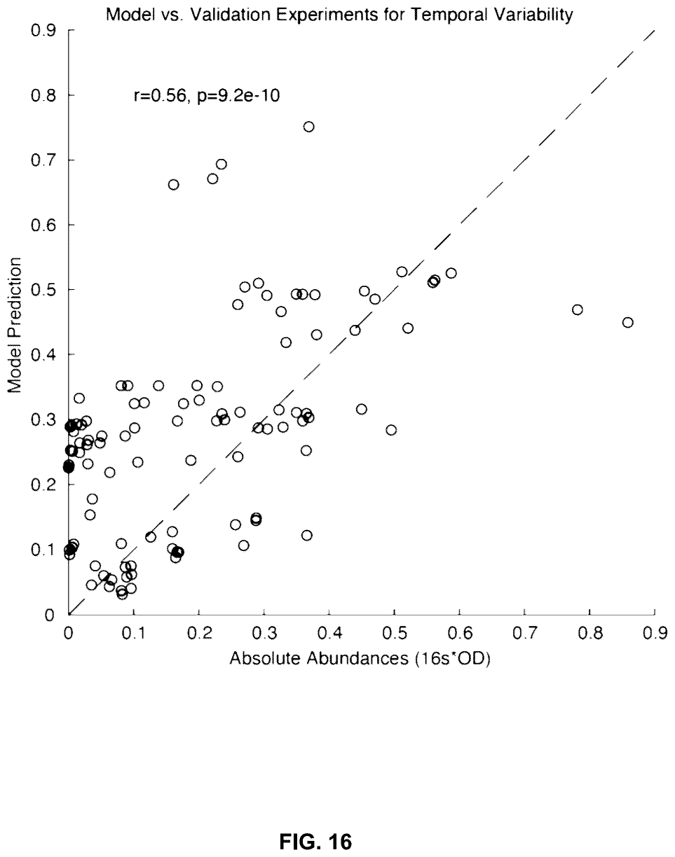

FIGS. 9A-9H. Tuning inoculum density as a control point for species composition. FIG. 9A. Overview: Design-test-learn cycle for maximizing community diversity as a function of inoculum densities. Model-guided design of inoculum densities (design), anaerobic coculture and DNA sequencing (test), and mapping endpoint growth to inoculum conditions (learn) are repeated to iteratively improve model accuracy and community diversity. FIG. 9B. Design: Inoculum density setpoints corresponding to experimental design center point levels across design-test-learn cycles. The center point of the experimental design represents the set of inoculum conditions expected to yield the highest diversity given the information that was available at the time. Species are sorted left to right by the magnitude of the difference between the log transformed inoculum densities of the first and third cycles. The dotted line indicates the even inoculum baseline condition. FIG. 9C. Test: Endpoint (28+/−1 hour) species compositions resulting from experimental designs of inoculum levels, sorted left to right by Shannon diversity. Stacked bars and error bars represent mean and standard deviation of three replicates, respectively, of a particular inoculum condition. Species indicated by the partitions in the stacked bars (from bottom to top) are BH, BL, BU, CA, DL, EL, ER, FP, PC, and PJ. FIG. 9D. Multivariate polynomial regression models (linear regression with interaction and quadratic terms) are trained to predict each species' community abundance from the inoculum design matrix. Test predictions (withheld data), Pearson correlation coefficient (rho), and p-value (p) are shown for the models trained on data from the first two DTL cycles. Validation (out-of-fold) predictions with species specific correlation coefficients are shown in FIGS. 10A-10B. FIG. 9E. Distributions of Shannon diversities (calculated from mean composition of three biological replicates) across design cycles. Dashed line indicates maximum possible Shannon diversity for a 10-member community. Dotted line indicates diversity from even inoculum on optimized medium. FIG. 9F. Benchmarking overall diversity improvements from media optimization (FIGS. 6A-6F) and inoculum optimization (FIGS. 9A-9H). Species composition (stacked bars, left-axis) and Shannon diversities as calculated from mean of species abundances (solid bar, right-axis); diamonds show diversities calculated from individual sets of replicates (right-axis). Even inoculum and baseline medium are indicated with (−), while (+) indicates that the community resulted from optimization efforts in this study. FIG. 9G. Log-Log scatter plot of the inoculum densities predicted by the CSLE model (DTL1 center point levels) vs. the experimentally identified best inoculum (condition yielding highest diversity after three DTL cycles). Pearson correlation is calculated between the logarithm of the inoculum densities, corresponding to log-log axis. FIG. 9H. Scale up of sample inoculum condition from 200 μL plate to 100 mL flask.

FIGS. 10A-10B. Regression models for DTL inoculum tuning cycles. FIG. 10A. (a) Validation (out-of-fold) predictions from regression models trained on data from DTL cycle one only. Species specific Pearson correlation and p-value are indicated in the legend. (b) Validation (out-of-fold) predictions from regression models trained on data from DTL cycles one and two. FIG. 10B. Bar plot indicates improvement of Pearson correlation coefficients after adding cycle two training data. Darker left-hand bars indicate cycle one correlations (as shown in panel a legend of FIG. 10A). Lighter, right-hand bars indicate cycle two correlations (as shown in panel b legend of FIG. 10A).

FIG. 11. Graphical Representation of the “frameshift” rational approach to assignment of new inoculum setpoints to the scaled high/center point/low design levels. The left panel shows a hypothetical two-species, two-inoculum level design (with a center point). The x-axis is the species 1 scaled level, the y-axis is the species two level, zeros and ones indicate the scaled levels (high and low). This hypothetical experiment results in a community composition in which neither species' growth response changes with the design levels: Species one because of lower than measurable growth, species two because of saturating overgrowth. This is qualitatively representative of the first DLT cycle, in which many species undergrew, and ER overgrew. Since the response is not measurably correlated with the design variables, a model cannot be fit. Rather, we update the levels by a half “frameshift” of the design levels: the new design uses the extremum of the old design as its new center point (low if overgrowth, high if undergrowth). The new design thus overlaps with the old design, and the new “center point” level that was just assigned can be used as an input to any regression model that was successfully trained, and used to predict interaction terms for the modeled species' optimal setpoint. The hypothetical updates design results in the species responses being correlated with design conditions, which is qualitatively representative of the second inoculum experiment. This approach flexibly integrates both experimental intuition and model-guided design. If modeling resources are not available, we expect this rational approach could be used to conduct a design-test-learn cycle without computational modeling.

FIGS. 12A-12D. Passaging training data for gLV model training. Relative abundance data for the three passages of DTL cycle 1 (FIGS. 12A-12C) and the one passage of cycle 3 (FIG. 12D). Inoculum design conditions are plotted on the x-axes. Species indicated by the partitions in the stacked bars (from bottom to top) are Blautia hydrogenotrophica (BH), Biffidobacterium longum (BL), Bacteroides unformis (BU), Collinsella aerofaciens (CA), Dorea longicatena (DL), Egerthella lenta (EL), Eubacterium rectale (ER), Faecalibacterium prausnitzii (FP), Prevotella copri (PC), and Parabacteroides johnsonii (PJ).

FIGS. 13A-13D. Hyperparameter selection, predictions, and parameters for the generalized Lotka-Volterra model. FIG. 13A. The L1 regularization coefficient is chosen as that which minimizes the average out-of-fold MSE and maximizes average out-of-fold Pearson correlation coefficient. This selection of regularization penalty suggests the best tradeoff between bias and variance. Small regularization parameters lead to overfitting of training data and poor prediction of test data, while large regularization coefficients identify solutions that prioritize small parameter magnitudes over data fits in order to minimize a lopsided cost function. FIG. 13B. Test predictions. Out-of-fold cross validation predictions are not shown, as they were only used to calculate mean MSE and correlation coefficient for hyperparameter selection. Test data is withheld from the entire fitting and hyperparameter selection process, meaning that predictions on this dataset are an unbiased metric for model predictivity. The species shading scheme is consistent with the rest of the drawings, the shapes of the markers represent the different experiments. FIG. 13C. Histogram of parameter values shown in heatmap in FIG. 13D. The two discussed parameters appear to be outliers. Many parameters are zero or near-zero because of the L1 regularization penalty. FIG. 13D. heatmap of interspecies interaction terms. The model's largest parameter explains ER's tendency to grow better in a community than in monoculture as a positive interspecies interaction with BH, while the smallest (largest negative) contributes to BL's tendency to undergrow in communities.

FIGS. 14A-C. The 33 unique solutions of the objective function for low temporal variability (methods). These result from all 967 simulated subcommunities of species number “10 choose k, k=3 through 9”. Many of the solutions converge to the same endpoint abundance, as a larger community where several strains are set to zero as initial conditions by the solver are equivalent to smaller communities that omit those species. The conditions with the asterisks were chosen for experimental validation. The “uniqetol” function in MATLAB is used to determine communities whose endpoint abundances were unique within a numerical tolerance. In each stacked bar, the listed microbes are shown in order from the bottom to the top of the bar.

FIGS. 15A-15D. Model-guided design of high and low temporal variability of species composition. FIG. 15A. Overview: a generalized Lotka-Volterra (gLV, equation 13) ordinary differential equation model is trained on monoculture timeseries and 10-member community initial conditions and endpoint data (methods). To design conditions with low temporal variability across the endpoint compositions of four simulated passages, an optimization algorithm maximizes the ratio of Shannon diversities to Euclidean distances as a function of inoculum density for all possible 3-to-9-member subcommunities. Nine low and three high temporal variability subcommunities are selected for experimental validation. FIG. 15B. Distribution of Euclidean distances between mean species compositions (n=3) of adjacent subcultures (1-2, 2-3, 3-4) for 10-member inoculum training data (passages of DTL cycle 1), designed low temporal variability subcommunities, and designed high temporal variability subcommunities. FIG. 15C. Computational predictions (first row), experimental validation results (second row), and gLV parameter networks (third row) for high temporal variability subcommunities. Endpoint relative abundances are plotted on the y-axis, passages 1 to 4 are plotted on the x-axis. Solid and dashed edges indicate negative and positive interaction parameters, respectively, scaled by magnitude. Node size is scaled by specific growth rate parameters. FIG. 15D. Computational predictions, experimental validation, and gLV parameter networks for low temporal variability subcommunities. In FIGS. 15C and 15D, the listed species in the third row are presented in the stacked bars of the first and second rows in alphabetical order from the bottom to the top of the bars.

FIG. 16. Scatter plot of model predictions vs. experimental validation for temporal variability communities. Y-axis is model predictions for the 9 low and 3 high variability communities, using the gLV model trained on monoculture data, 10-member community data (including 3 passages of the first DTL cycle and 1 passage of the third). Validation experiments contain 2-4 member communities over four passages.

Unless otherwise noted, the species represented in stacked bars in the drawings herein are presented in alphabetical order from the bottom to the top of the bar.

### DETAILED DESCRIPTION OF THE INVENTION

One aspect of the invention is directed to methods of designing production of a microbial community. The microbial community can comprise a set of microbes. The set of microbes can comprise any combination of microbes. Such microbes can include prokaryotes, eukaryotes, and combinations thereof. Exemplary prokaryotes include bacteria and archea. Exemplary eukaryotes include protozoa, fungi (such as yeasts), and algae.

The microbial community can be designed to comprise different microbe subsets of the set of microbes. “Different microbe subsets” refers to subsets of microbes within the set of microbes that can be detectably distinguished from each other. The microbe subsets can be defined by microbes being classified within any type of biological classification, including, for example, particular domains, kingdoms, phyla, classes, orders, families, genera, species, subspecies, strains, types of bacteria, types of eukaryotes, types of protozoa, types of fungi, types of yeast, types of algae, or any combination thereof. The differences between the microbe subsets can accordingly be defined as differences across any type of biological classification. In some cases, for example, the different microbe subsets may be defined as microbes from different domains. In some cases, the different microbe subsets may be defined as microbes from different kingdoms. In some cases, the different microbe subsets may be defined as microbes from different phyla. In some cases, the different microbe subsets may be defined as microbes from different classes. In some cases, the different microbe subsets may be defined as microbes from different orders. In some cases, the different microbe subsets may be defined as microbes from different families. In some cases, the different microbe subsets may be defined as microbes from different genera. In some cases, the different microbe subsets may be defined as microbes from different species. In some cases, the different microbe subsets may be defined as microbes from different subspecies. In some cases, the different microbe subsets may be defined as microbes from different strains. In some cases, the different microbe subsets may be defined as microbes from different types of bacteria. In some cases, the different microbe subsets may be defined as microbes from different types of eukaryotes. In some cases, the different microbe subsets may be defined as microbes from different types of protozoa. In some cases, the different microbe subsets may be defined as microbes from different types of fungi. In some cases, the different microbe subsets may be defined as microbes from different types of yeast. In some cases, the different microbe subsets may be defined as microbes from different types of algae. Any combination of the above-referenced definitions of the microbe subsets are also acceptable. For example, in some cases, one microbe subset may include eukaryotic microbes and the remaining microbe subsets may include different types of prokaryotic microbes.

In some versions, each microbe subset can comprise microbes from more than one classification type. In some versions, each microbe subset comprises microbes from no more than one classification type. For example, in some versions, each microbe subset comprises no more than one microbial family. In some versions, each microbe subset comprises no more than one microbial genus. In some versions, each microbe subset comprises no more than one microbial species.

The number of microbe subsets in the set can be any number greater than 1. In some versions, the set of microbes comprises from 2 to 1,000 microbe subsets, such as from 2 to 750, from 2 to 500, from 2 to 250, from 2 to 100, from 2 to 50, from 2 to 25, from 2 to 20, or from 2 to 15 microbe subsets.

The microbial community can be designed to comprise the different microbe subsets in a proportion approximating a target proportion. “Target proportion” refers to the relative abundance of each of the microbe subsets with respect to each other within the set of microbes. Exemplary methods of determining the relative abundance of different microbes are provided in the following examples. The relative abundances can be expressed by microbe number, OD 600, or other measurements of abundance. The term “approximating” in the phrase “a proportion approximating a target proportion” is used synonymously with “of about” or “of approximately” and encompasses the exact target proportion and slight differences therefrom, as the exact target proportion may be attainable in some but not all cases. In some versions, the target proportion is an equal relative abundance of each microbe subset. In other versions, the target proportion comprises an unequal relative abundance of at least some of the microbe subsets.

The method of designing production of the microbial community can comprise designing a target medium.

Designing the target medium can comprise a step of growing each of the microbe subsets separately to carrying capacity in each of multiple test media comprising different test-concentration profiles. “Carrying capacity” is used herein to refer to the population size of a given microbe subset at steady state and can be expressed as microbe number, OD 600, or any other suitable measure of the population size of a microbe. “Test-concentration profile” refers to a concentration profile to be tested. “Concentration profile” refers to a set of one or more medium components that is present in at least one of the test media at a different concentration than in at least one other of the test media. For the purposes herein, the absence of a particular medium component in a given test medium constitutes a concentration of the medium component so long as at least one other of the test media includes the particular medium component in at least some amount. The medium component(s) tested in a given set of test media can include any medium component desired to be tested, and can include carbohydrates (including sugars, oligosaccharides, polysaccharides), amino acids, polypeptides, lipids, salts, pH, small molecules, vitamins, minerals, animal digests, yeast digests, and/or any other agent that can be included in a medium. For the purposes herein, pH is considered to be a medium component insofar as it is a measure of proton concentration. In various versions of the invention, the concentration profile can comprise at least 1, at least 2, at least 3, at least 4, or at least 5 medium components and/or up to 6, up to 7, up to 8, up to 9, up to 10, up to 15, up to 20, up to 25, up to 30, up to 35, up to 40, up to 45, or up to 50, or more medium components.

Designing the target medium can further comprise determining the proportion of microbe subset carrying capacities in each test medium to thereby obtain test-medium proportions corresponding to the test-concentration profiles. “Test-medium proportion” refers to the relative abundances of the individual subsets at carrying capacity in a given test medium as proportions of the total sum of all the abundances of the individual subsets at carrying capacity in the given test medium. The correspondence of the test-medium proportions to the test-concentration profiles occurs by virtue of the test-concentration profiles comprised by a given test medium in which a given test-medium proportion is determined.

Designing the target medium can further comprise determining a target-concentration profile from the test-medium proportions and the corresponding test-concentration profiles. The target-concentration profile is preferably one that yields or is predicted to yield a target-medium proportion of microbe subset carrying capacities that differs from at least one of the test-medium proportions. The target medium can then be defined as comprising the target-concentration profile. “Target-concentration profile” refers to a particular concentration profile determined from the test-medium proportions and the corresponding test-concentration profiles. “Target-medium proportion” refers to an actual or predicted proportion of the microbe subsets at carrying capacity in a medium comprising the target-concentration profile. As outlined in further detail below, the determination of the target-concentration profile can occur through selecting a test-concentration profile that yields a favorable test-medium proportion or through extrapolating from the test-medium proportions and the corresponding test-concentration profiles to predict a target-concentration profile that yields a favorable predicted target-medium proportion.

For the purposes of obtaining a suitable target medium, it is preferred that the target-medium proportion is a proportion more similar to the target proportion than at least one of the test-medium proportions. In some versions, the target-medium proportion is a proportion more similar to the target proportion than each of the test-medium proportions.

Mathematical metrics for quantifying the similarity between various sets of relative abundances are well known in the art. A mathematical metric for determining similarity of a given set of relative abundances with respect to a target set of equal relative abundances, for example, is Shannon diversity. Metrics for determining similarity of a given set of relative abundances with respect to a target set of unequal relative abundances are also known and can be used in the present methods. Any mathematical relationship that relates the set of (regression) model outputs to the desired target proportion, such that when this mathematical relationship is maximized/minimized as a function of input variables (e.g. media components), can be used to predict a set of inputs yielding a target-profile that is most similar to the target-proportion. Shannon diversity of carrying capacities is just one example, wherein this function is maximized for an even species abundance of all species. The mean squared error (MSE) can also be minimized between a vector of target species proportions and a vector of predicted species proportions (see the following examples with regard to inoculum optimization). The Euclidean distance between a vector of target proportions and a vector of predicted species proportions can also be minimized. Any mathematical function that relates the predicted abundances of all species to desired abundances and can be minimized/maximized can be used. Exemplary off-the-shelf optimization solvers for use of such functions includes “fmincon” or “scipy minimize.”

In some versions, determining the target-concentration profile comprises selecting a test-concentration profile yielding a suitable test-medium proportion. In such a case, the target-concentration profile is one of the test-concentration profiles.

In other versions, determining the target-concentration profile comprises using the test-medium proportions and the corresponding test-concentration profiles to obtain a target-concentration profile that is not one of the test-concentration profiles. Such a version can comprise modeling the test-medium proportions and the corresponding test-concentration profiles to obtain predicted-carrying-capacity proportions of the microbe subsets and corresponding predicted-concentration profiles. The target-concentration profile can then be selected from one of the predicted-concentration profiles. The modeling in such versions can include regression modeling. Exemplary regression models are provided in the following examples. It is preferred that the target-concentration profile is one of the predicted-concentration profiles corresponding to one of the predicted-carrying-capacity proportions with maximized similarity to the target proportion. In versions in which the target proportion is an equal relative abundance of each microbe subset, the target-concentration profile can be one of the predicted-concentration profiles corresponding to one of the predicted-carrying-capacity proportions with maximized Shannon diversity.

The method of designing production of the microbial community can alternatively or additionally comprise designing a target-inoculum-density profile. “Target-inoculum-density profile” refers to a set of target inoculum densities of the microbe subsets in a given relative proportion that can be used for inoculating a given medium. “Inoculum density” refers to an amount of a microbe subset used to inoculate a medium.

Designing the target-inoculum-density profile can comprise growing to stationary phase the microbe subsets together in each of multiple inoculum test cultures inoculated with different test-inoculum-density profiles of the microbe subsets. “Test-inoculum-density profile” refers to a set of inoculum densities of the microbe subsets in a given relative proportion for testing.

Designing the target-inoculum-density profile can further comprise determining the proportion of the microbe subsets at stationary phase in each inoculum test culture to thereby obtain test-inoculum-density proportions corresponding to the test-inoculum-density profiles. “Test-inoculum-density proportion” refers to the relative abundances of the individual subsets at stationary phase when grown together in a given inoculum test culture. The correspondence of the test-inoculum-density proportions to the test-inoculum-density profiles occurs by virtue of the test-inoculum-density profiles comprised by a given inoculum test culture in which a given test-inoculum-density proportion is determined.

Designing the target-inoculum-density profile can further comprise determining the target-inoculum-density profile from the test-inoculum-density proportions and the corresponding test-inoculum-density profiles. The target-inoculum-density profile is preferably one that yields or is predicted to yield a target-inoculum-density proportion of the microbe subsets that differs from at least one of the test-inoculum-density proportions. “Target-inoculum-density profile” refers to a particular target inoculum profile determined from the test-inoculum-density proportions and the corresponding test-inoculum-density profiles. “Target-inoculum-density proportion” refers to an actual or predicted proportion of the microbe subsets at stationary phase in a medium inoculated with the microbe subsets in the target-inoculum-density profile. As outlined below, the determination of the target-inoculum-density profile can occur through selecting a test-inoculum-density profile that yields a favorable test-inoculum-density proportion or through extrapolating from the test-inoculum-density proportions and the corresponding test-inoculum-density profiles to predict a target-inoculum-density profile that yields a favorable predicted target-inoculum-density proportion.

For the purposes of obtaining a suitable target-inoculum-density profile, it is preferred that the target-inoculum-density proportion is a proportion more similar to the target proportion than at least one of the test-inoculum-density proportions. In some versions, the target-inoculum-density proportion is a proportion more similar to the target proportion than each of the test-inoculum-density proportion.

In some versions, determining target-inoculum-density profile comprises selecting a test-inoculum-density profile yielding a suitable test-inoculum-density proportion. In such a case, the target-inoculum-density profile is one of the test-inoculum-density profiles.

In other versions, determining the target-inoculum-density profile comprises using the test-inoculum-density proportions and the corresponding test-inoculum-density profiles to obtain a target-inoculum-density profile that is not one of the test-inoculum-density profiles. Such a version can comprise modeling the test-inoculum-density proportions and the corresponding test-inoculum-density profiles to obtain predicted-steady-state proportions of the microbe subsets and corresponding predicted-inoculum-density profiles. The target-inoculum-density profile can then be selected from one of the predicted-inoculum-density profiles. The modeling in such versions can include regression modeling. Exemplary regression models are provided in the following examples. It is preferred that the target-inoculum-density profile is one of the predicted-inoculum-density profiles corresponding to one of the predicted-steady-state proportions with maximized similarity to the target proportion. In versions in which the target proportion is an equal relative abundance of each microbe subset, the target-inoculum-density profile can be one of the predicted-inoculum-density profiles corresponding to one of the predicted-steady-state proportions with maximized Shannon diversity.

The steps of designing the target-inoculum-density profile outlined above can be performed in an iterative fashion. The target-inoculum-density profile of a prior iteration in such a case can be used to design the test-inoculum-density profiles of a subsequent iteration. In some versions, for example, the test-inoculum-density profiles of a subsequent iteration can be determined as points encompassing in a multivariate design space the target-inoculum-density profile in a prior iteration. In some versions, the target-inoculum-density profile in the prior iteration serves as a center reference point in the multivariate design space. An exemplary iterative performance of steps in designing the target-inoculum-density profile as outlined above is provided as the “design-test-learn” cycle in the following examples.

Regardless of whether the steps of designing the target-inoculum-density profile outlined above are performed in an iterative fashion, initial-test-inoculum-density profiles can be designed. “Initial-test-inoculum-density profile” refers to a test-inoculum-density profile employed in a first performance of designing the target-inoculum-density profile. The design of the initial-test-inoculum-density profiles can comprise growing each of the microbe subsets separately in a set of first inoculum test cultures and measuring kinetic growth of each microbe subset in its first inoculum test culture. “Kinetic growth” in this context refers to the growth as a function of time.

The design of the initial-test-inoculum-density profiles can further comprise growing each of the microbe subsets together in a second inoculum test culture and measuring total growth limit of the microbe subsets in the second inoculum test culture. “Total growth limit” in this context refers to saturated upper limit of growth of total microbes in a given culture.

The design of the initial-test-inoculum-density profiles can further comprise modeling the kinetic growth of each microbe subset and the total growth limit to determine a reference-inoculum-density profile. “Reference-inoculum-density profile” refers to an inoculum-density profile predicted to yield an initial-target-inoculum-density proportion, wherein the initial-target-inoculum-density proportion is a predicted relative abundance of the microbe subsets at steady state when grown together. It is preferred that the initial-target-inoculum-density proportion is a proportion predicted in the modeling of step (3c) as having maximized similarity to the target proportion. In versions in which the target proportion is an equal relative abundance of each microbe subset, the initial-target-inoculum-density proportion can be a proportion predicted in the modeling of step (3c) as having maximized Shannon diversity.

The design of the initial-test-inoculum-density profiles can further comprise determining the initial-test-inoculum-density profiles as points encompassing the reference-inoculum-density profile in a multivariate design space. In some versions, the reference-inoculum-density profile serves as a center reference point in the multivariate design space.

In some versions of the invention, the method of designing the production of the microbial community comprises designing a target medium as well as designing a target-inoculum-density profile. In such cases, its preferred that the designing of the target medium is performed prior to the designing of the target-inoculum-density profile. It is further preferred that some or all of various growth steps in the designing of the target-inoculum-density profile are performed with the target medium. These steps include growing to stationary phase the microbe subsets together in each of the multiple inoculum test cultures inoculated with the different test-inoculum-density profiles of the microbe subsets, the growing of each of the microbe subsets separately in the set of first inoculum test cultures, and/or the growing of each of the microbe subsets together in the second inoculum test culture.

Some versions of the invention comprise designing a target medium as well as designing a target-inoculum-density profile for production of an microbial community comprising an unequal target proportion of community members (e.g., the microbe subsets of the set of microbes). In such versions, the target-medium proportion can be targeted for increased (e.g., maximized) similarity for an equal proportion (e.g., maximizing Shannon diversity) of the microbe subsets to ensure growth, and the target-inoculum-density profile can be targeted for increased (e.g., maximized) similarity to the unequal target proportion as the ultimate target proportion. The target-medium proportion, for example, can be a proportion more similar to an equal target proportion than at least one, some, or each, of the test-medium proportions. Furthermore, the target-inoculum-density proportion can be a proportion more similar to the unequal proportion as the ultimate target proportion than at least one, some or each, of the test-inoculum-density proportions.

The target medium and/or target-inoculum-density profile of the microbe subsets can be used in a method of producing a microbial community. The method can comprise inoculating a microbial community medium with microbe subsets of a set of microbes and growing the microbe subsets. In some versions, the microbial community medium is the target medium. In some versions, the microbial community medium is inoculated with the microbe subsets in the target-inoculum-density profile. In some versions, the microbial community medium is the target medium, and the microbial community medium is inoculated with the microbe subsets in the target-inoculum-density profile. In some versions, the set of microbes comprises from 2 to 1,000 microbe subsets, such as from 2 to 750, from 2 to 500, from 2 to 250, from 2 to 100, from 2 to 50, from 2 to 25, from 2 to 20, or from 2 to 15 microbe subsets. In some versions, the microbe subsets are grown to steady state.

In various versions, the microbe subsets have a proportion with a Shannon diversity value greater than 1.5 at steady state, such as greater than 1.6, greater than 1.7, greater than 1.8, greater than 1.9, greater than 2, greater than 2.1, greater than 2.2, greater than 2.3, greater than 2.4, greater than 2.5, greater than 2.6, greater than 2.7, greater than 2.8, greater than 2.9, greater than 3.0, greater than 3.1, greater than 3.2, greater than 3.3, greater than 3.4, greater than 3.5, greater than 3.6, greater than 3.7, greater than 3.8, greater than 3.9, greater than 4.0, greater than 4.1, greater than 4.2, greater than 4.3, greater than 4.4, greater than 4.5, greater than 4.6, greater than 4.7, greater than 4.8, greater than 4.9, greater than 5.0, greater than 5.1, greater than 5.2, greater than 5.3, greater than 5.4, greater than 5.5, greater than 5.6, greater than 5.7, greater than 5.8, greater than 5.9, greater than 6.0, greater than 6.1, greater than 6.2, greater than 6.3, greater than 6.4, greater than 6.5, greater than 6.6, greater than 6.7, or greater than 6.8 at steady state.

In various versions, the microbe subsets have a proportion with a Shannon diversity value up to 2, up to 2.1, up to 2.2, up to 2.3, up to 2.4, up to 2.5, up to 2.6, up to 2.7, up to 2.8, up to 2.9, up to 3.0, up to 3.1, up to 3.2, up to 3.3, up to 3.4, up to 3.5, up to 3.6, up to 3.7, up to 3.8, up to 3.9, up to 4.0, up to 4.1, up to 4.2, up to 4.3, up to 4.4, up to 4.5, up to 4.6, up to 4.7, up to 4.8, up to 4.9, up to 5.0, up to 5.1, up to 5.2, up to 5.3, up to 5.4, up to 5.5, up to 5.6, up to 5.7, up to 5.8, up to 5.9, up to 6.0, up to 6.1, up to 6.2, up to 6.3, up to 6.4, up to 6.5, up to 6.6, up to 6.7, up to 6.8, or up to 6.9 at steady state.

The invention accordingly also provides microbial communities generated with the methods described herein. The microbial communities can comprise a set of microbes comprising microbe subsets in a defined target proportion. The microbial community can be present in a composition. The composition can comprise the microbial community at a total growth limit. In some versions, the set of microbes comprises from 2 to 1,000 microbe subsets, such as from 2 to 750, from 2 to 500, from 2 to 250, from 2 to 100, from 2 to 50, from 2 to 25, from 2 to 20, or from 2 to 15 microbe subsets. In some versions, the microbe subsets have a proportion with a Shannon diversity value greater than 1.5, such as greater than 1.6, greater than 1.7, greater than 1.8, greater than 1.9, or greater than 2.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Bioprocess Design Strategies for Producing Microbial Consortia of Defined Phylogenetic Composition in Single-Vessel Community Culture

### SUMMARY

Microbial communities have tremendous potential as human therapeutics. However, manufacturing high-diversity microbial communities with desired species compositions is challenging. We develop a two-stage, model-guided framework to culture defined microbial communities at target species compositions. We apply this method to optimize the species diversity of a synthetic human gut community. The first stage exploits media components to optimize the growth of individual species and the second stage uses the initial abundance of each species as a control point to manipulate community composition. Using a design-test-learn cycle, we achieve 91% of the maximum possible community diversity. Leveraging these data, we then construct a dynamic ecological model to guide the design of communities with desired temporal properties. In sum, a deeper understanding of how microbial community assembly responds to changes in media formulation, initial species abundances, and inter-species interactions can enable the predictable design of high-diversity communities with desired compositions.

### Introduction

The potential of microbial communities as human therapeutics is evidenced by the remarkable efficacy of fecal microbiota transplantation (FMT) in treating recurrent C. difficile infection¹. This strategy of modifying a patient's dysbiotic microbiome with live, therapeutic organisms (“bugs-as-drugs”) holds significant promise for treating an ever-lengthening list of microbiome associated health conditions². However, FMT also poses the risk of pathogen transmission and other adverse health outcomes³⁻⁵. Further issues with this procedure include development of regulatory standards, definition of a precise mechanism of action, and scalability of donor material supply chain^{6,7}. A promising alternative is the use of defined microbial community therapeutics⁸. These well-characterized mixtures of cultured strain isolates could be optimized for beneficial properties while avoiding drawbacks of FMT⁹⁻¹¹.

A key challenge towards this goal is the scalable production of defined, therapeutic communities that span the phylogenetic and functional diversity of the healthy adult microbiome¹². Most of the commercially successful “probiotics” that are commonly recommended by physicians have gained traction not because of conclusive clinical indications, but rather because they are relatively easy to produce¹³. “Probiotics” tend to be oxygen-tolerant anaerobes like Lactobacilli and Bifidobacterium, while the healthy adult microbiome tends to be dominated by fastidious, oxygen-sensitive anaerobes such as Bacteroides, Prevotella, Clostridiaceae, Ruminococcacae, and Lachnospiricae¹⁴. “Probiotics” have even been shown to impair post-antibiotic microbiome recovery¹⁵. The challenge of producing therapeutic communities is a barrier to more than just commercial manufacturing; it slows scientific progress by limiting pilot-scale drug supply to clinical trials¹³. It also precludes use of therapeutic communities for low-cost, global health applications^{16,17}. A major contribution to this production challenge is the current strain culturing process, in which each of the many constituent organisms of the community are grown as separate cultures, then subsequently mixed to a desired species composition¹⁷. This process is complicated, costly, and scales poorly for communities with large numbers of organisms¹⁷. New methods to predictably culture microbial communities with desired species compositions could alleviate this manufacturing bottleneck.

Developing model-guided approaches to predict community growth as a function of specific control inputs would greatly enhance our ability to manipulate community composition towards a desired state¹⁸. Design of experiments with statistical modeling (DoE) is increasingly used to optimize complex biological systems, for example modulating protein translation as a function of synthetic DNA sequence or tuning gene expression to improve efficiency of an engineered metabolic pathway.¹⁹⁻²¹ This generalizable framework has also been used to design chemically defined media by optimizing microbial growth as a function of various media components^{22,23}. Statistical modeling, an integral part of the DoE workflow, has been applied to predict microbial community composition as a function of dietary inputs, though it is more commonly used to predict community-level function from species abundance²⁴⁻²⁶. Dynamic ecological models, while generally lacking abiotic control points like resources, are predictive of microbial community assembly in a particular media environment^{27,28}. These population-based studies demonstrate that inter-species interactions and initial species abundances strongly affect transient states of community assembly. We exploit both key media components and initial population densities as control points for a synthetic human gut community. Design of experiments, statistical models, and dynamic ecological models are used to predict how simultaneous adjustments to multiple control inputs affects the resulting community composition.

We develop a two-stage, model-guided approach for systematically tuning these control points towards achieving our target system output: a synthetic human gut community culture with high endpoint species diversity. First, we design a new culture medium that yields similar endpoint abundances of all species in monoculture, and further demonstrate that this monoculture-based optimization improves community diversity. Then, in communities cultured on the new medium, we use a design-test-learn cycle to modulate individual species' initial population densities (i.e. inocula) towards setpoints that yield our target output. In both stages, a substantial degree of community composition (a systems-level property) can be forecasted as the composite behavior of constituent monocultures (parts-level properties)²⁹. Finally, we use our data to build a dynamic ecological model that captures inter-species interactions, and use this model to guide the design of communities with distinct classes of dynamic behaviors. In sum, we demonstrate that computational models coupled to control of a limited set of key media factors and initial species densities is a powerful approach for steering microbial communities to desired states.

### Results

Manipulating Key Media Components to Enhance Community Shannon Diversity We aimed to maximize the Shannon diversity (equation 1) of a synthetic human gut community. Shannon diversity is an ecological metric used to characterize both the number of species in a community and the evenness of their population sizes³⁰. We pre-selected a representative set of 10 human gut species that span the phylogenetic and metabolic diversity of the human gut microbiome. The community included Blautia hydrogenotrophica (BH), Biffidobacterium longum (BL), Bacteroides unformis (BU), Collinsella aerofaciens (CA), Dorea longicatena (DL), Egerthella lenta (EL), Eubacterium rectale (ER), Faecalibacterium prausnitzii (FP), Prevotella copri (PC), and Parabacteroides johnsonii (PJ). Several of these species have been shown to be critical to the recovery of a healthy microbiome after childhood malnutrition³¹. In particular, Faecalibacterium prausnitzii has been extensively associated with therapeutic properties³².

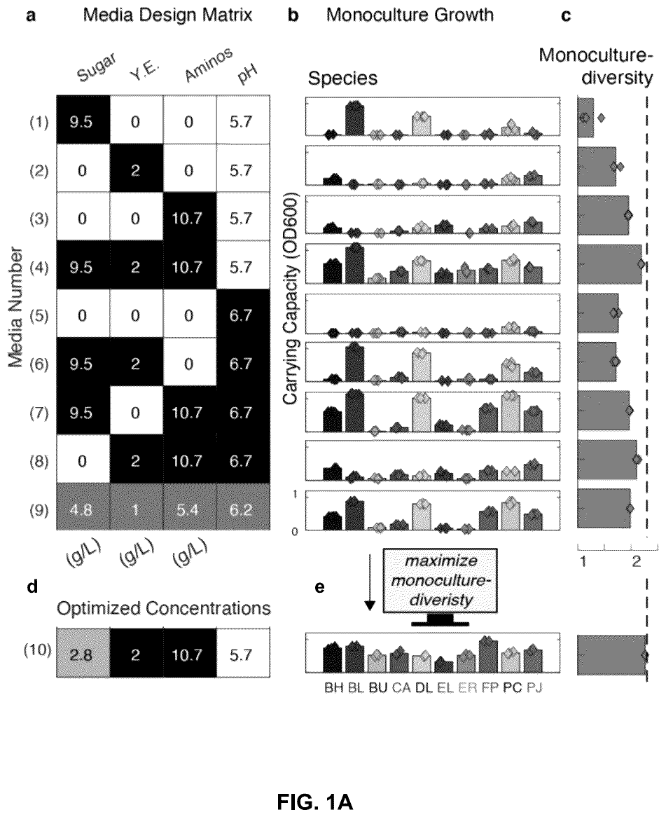

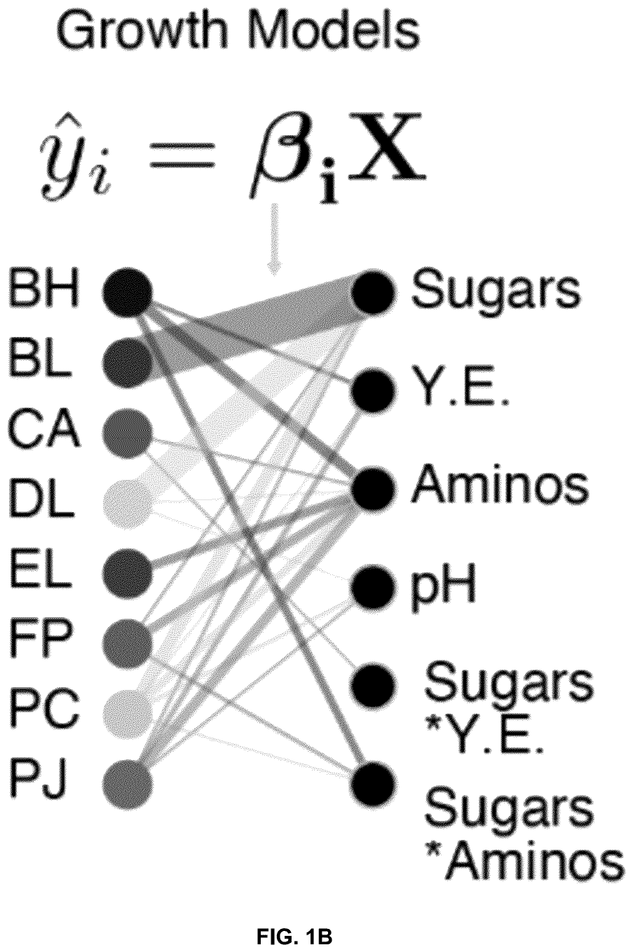

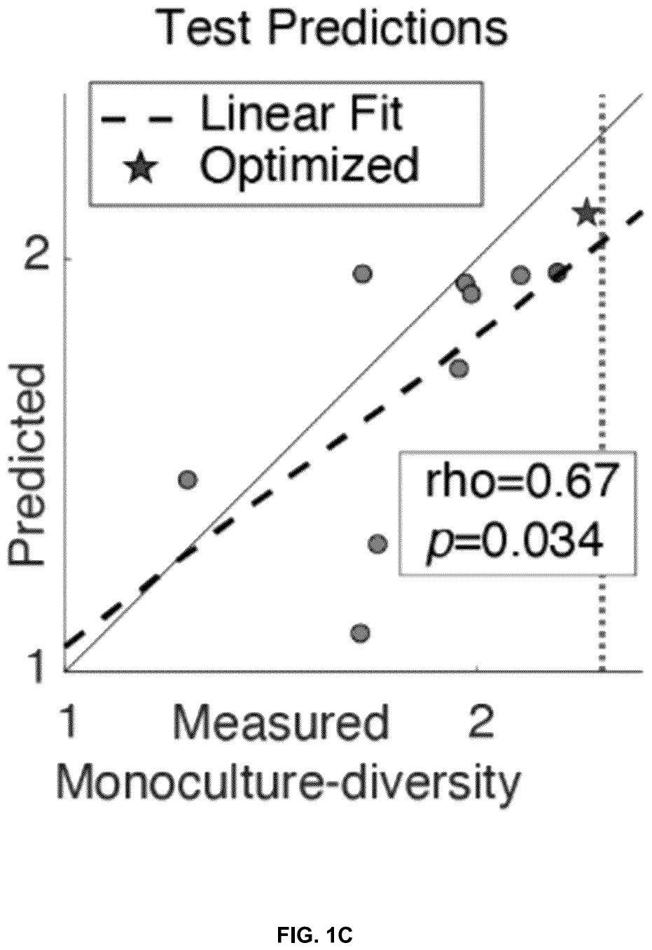

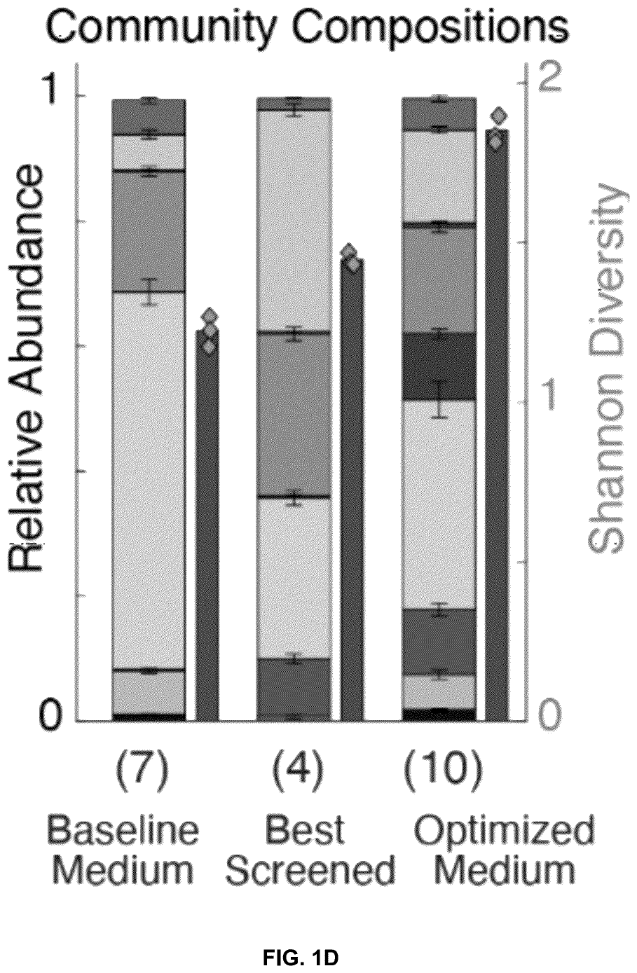

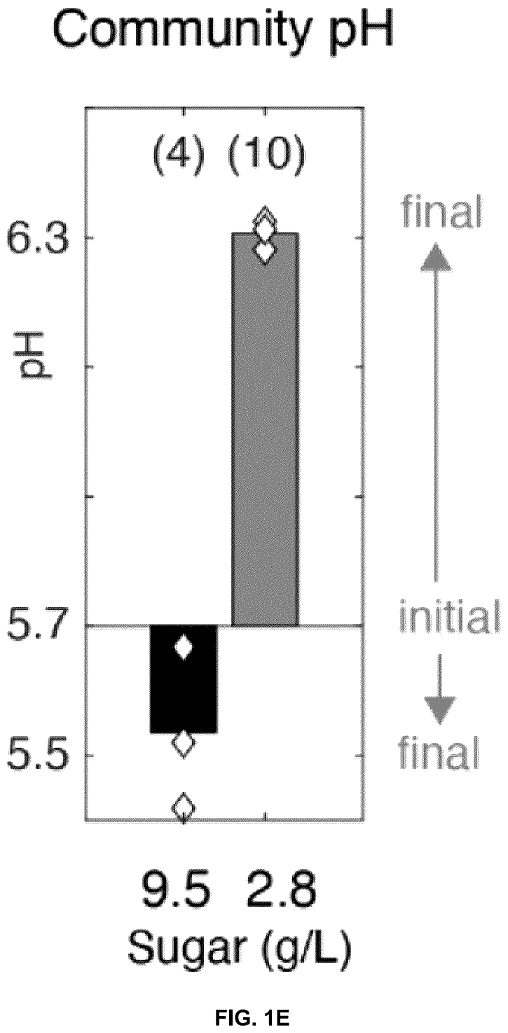

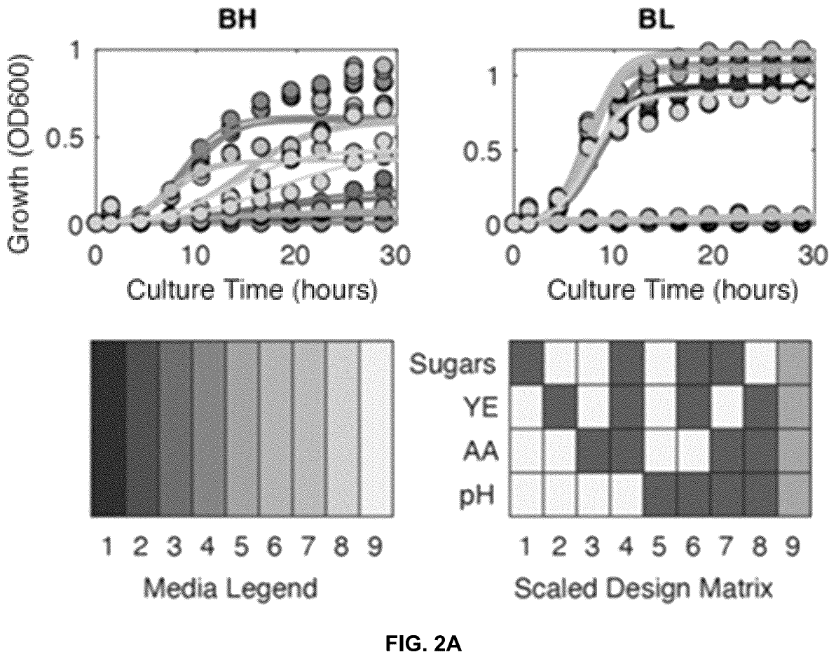

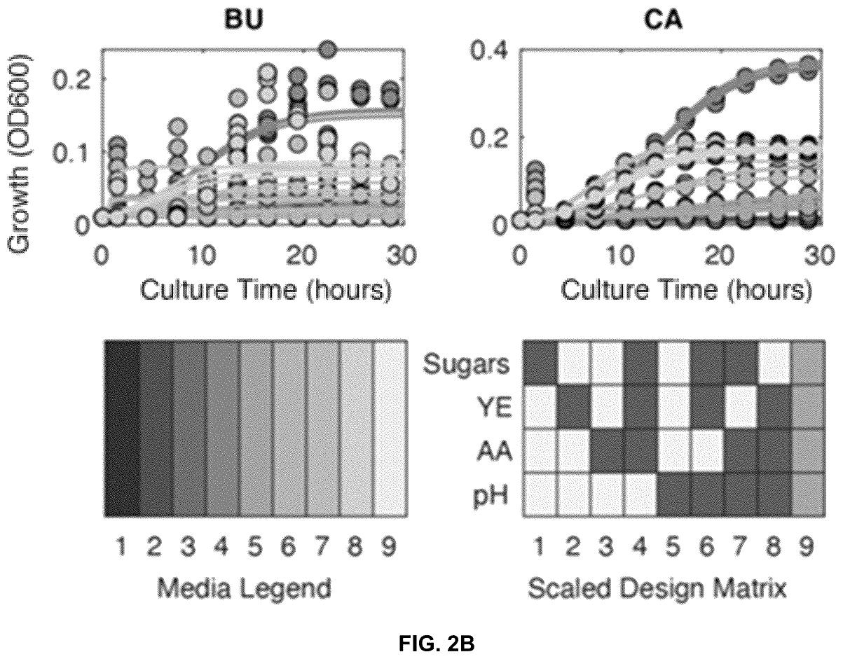

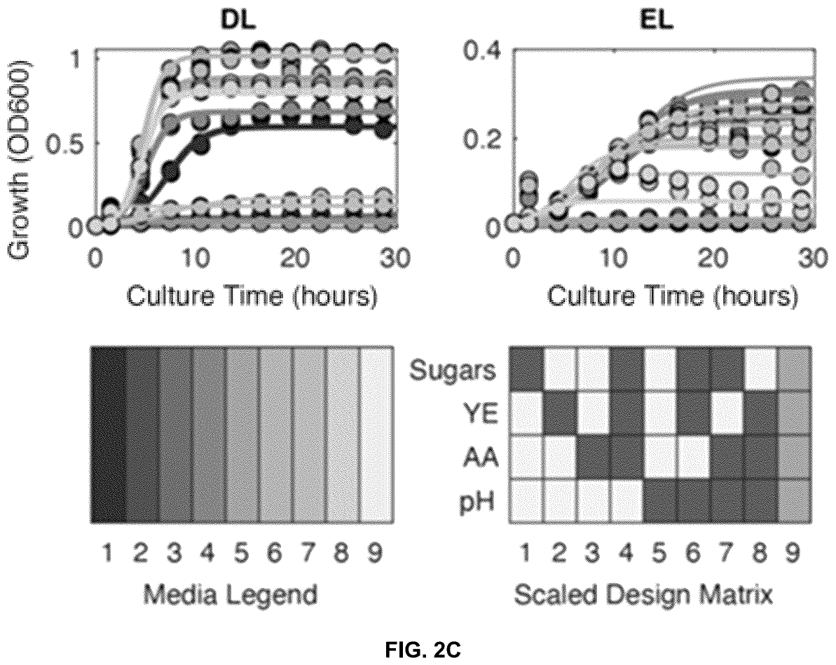

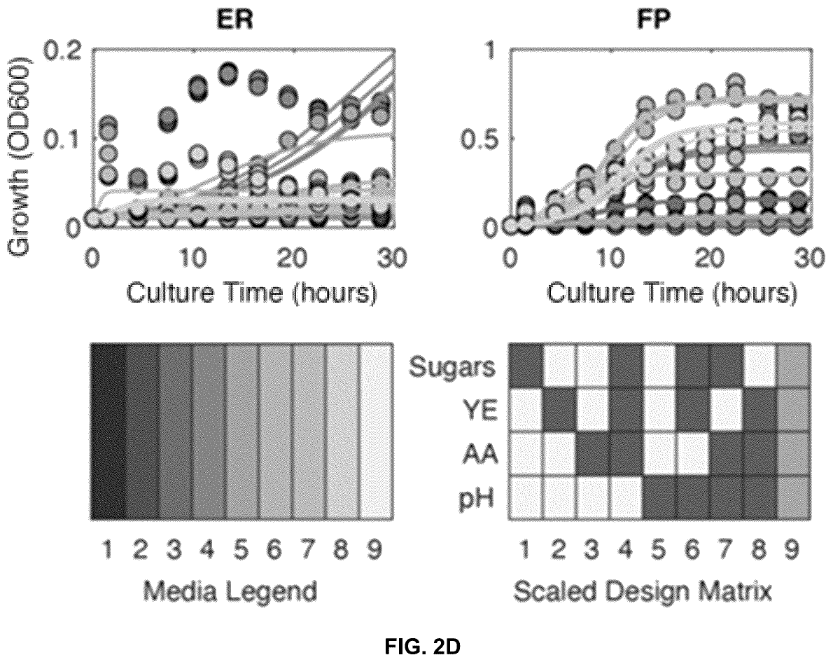

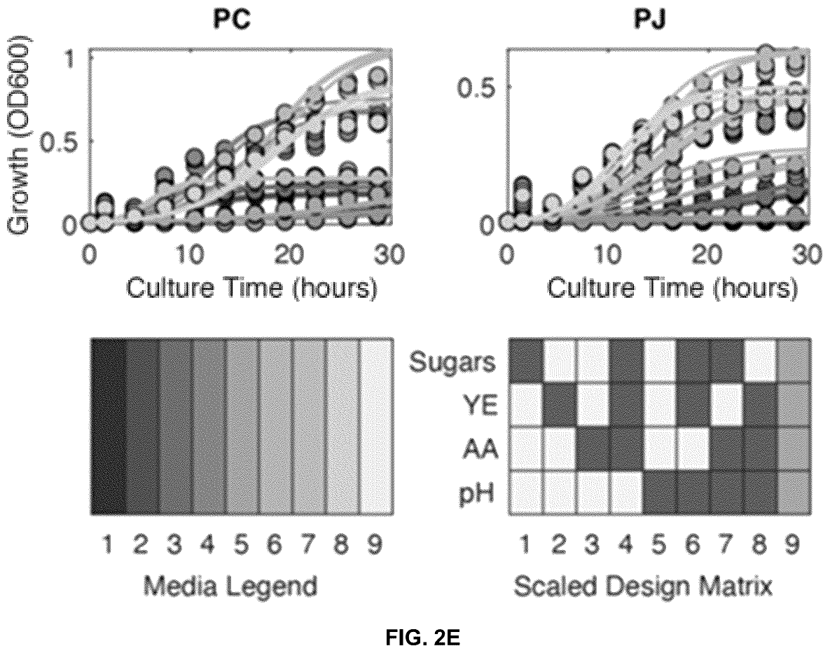

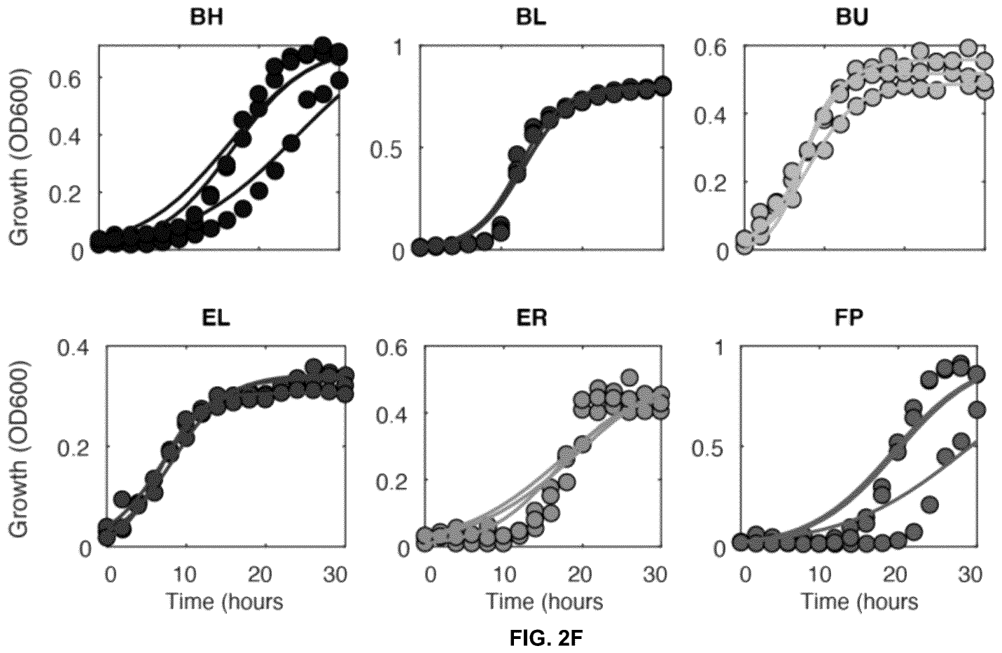

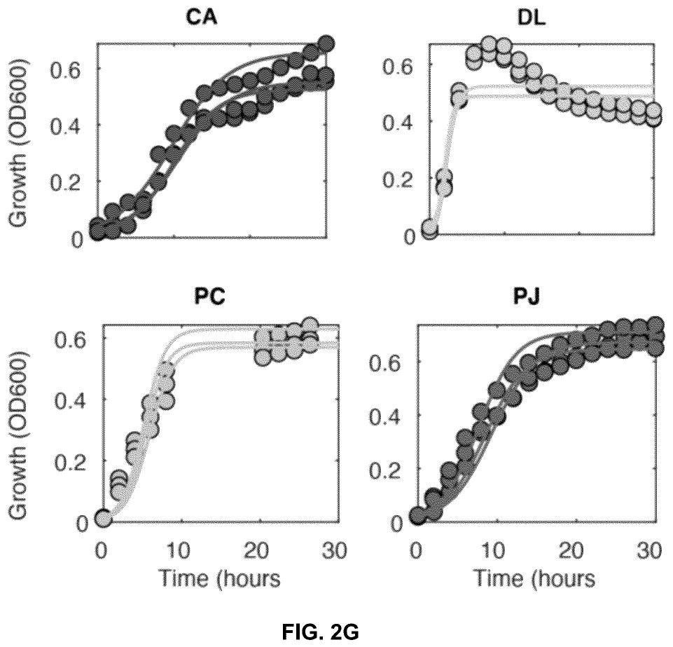

We characterized the growth of each individual species (monoculture) in a baseline defined medium similar to that which was previously used to grow a diverse set of human gut microbiome species (Methods)²⁴. The monocultures displayed a wide range of growth rates and population sizes at steady state (i.e. carrying capacities) (FIGS. 1A and 2A-2G, medium 7). Human gut anaerobes have diverse metabolic strategies^{33,34}. Therefore, we exploited the concentrations of key media components to manipulate individual species growth responses^{33,35}. Sugars and amino acids represented the main fermentable substrates in the baseline medium, and are important resources in the mammalian gut³⁶. Likewise, pH is a major environmental factor, and can distinctly modify the growth of different organisms³⁷. In addition, we selected yeast extract since it comprises a complex digest containing vitamins, peptides, and other resources, and supports the growth of F. prausnitzii³⁸. We used statistical design of experiments (DoE) to identify an optimal concentration profile of these components by manipulating four key variables: (1) a mixture of three sugars, (2) a defined mixture of amino acids, (3) yeast extract, and (4) pH. The optimal media profile was defined as one that maximized similarity between steady-state population sizes of individual species (monocultures), while also supporting sufficient total growth (FIG. 1A, Methods).³⁹

We characterized the monoculture growth responses in each media condition (FIG. 1A) and fit a logistic growth model to time-series optical density measurements at 600 nm (OD600) (FIGS. 2A-2G). The carrying capacity parameter of this model indicates population size at steady state (FIG. 1A, panel b). To quantify the similarity among the growth responses of individual species as a function of media components, we calculated the Shannon diversity of the normalized carrying capacities in a particular medium (equation 4). This quantity, hereafter referred to as “monoculture-diversity,” varied widely as a function of media composition (FIG. 1A, panel c).

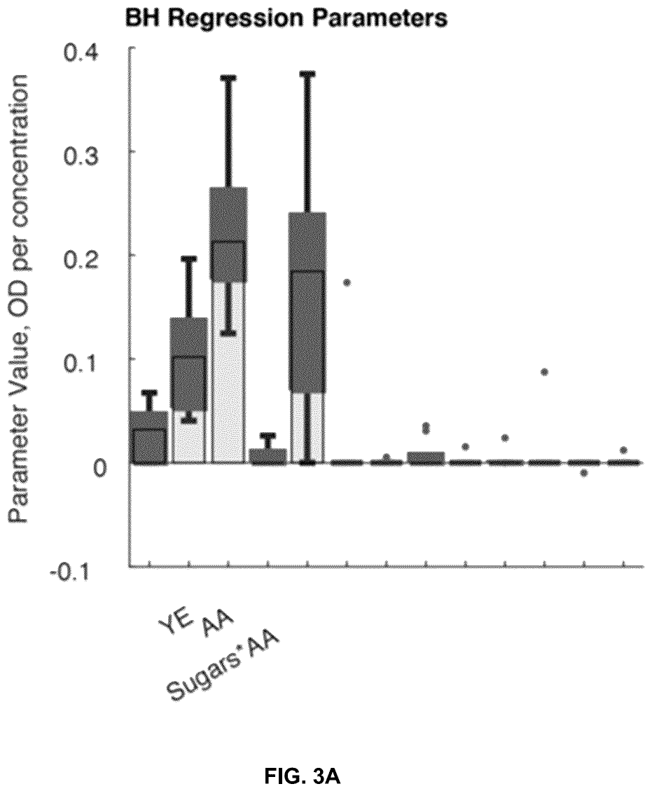

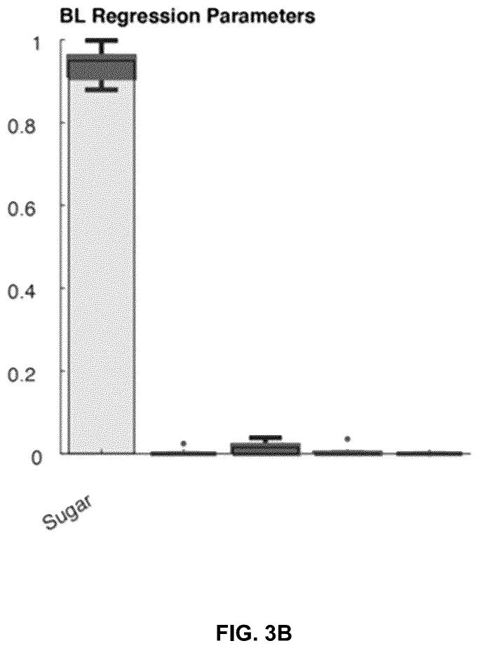

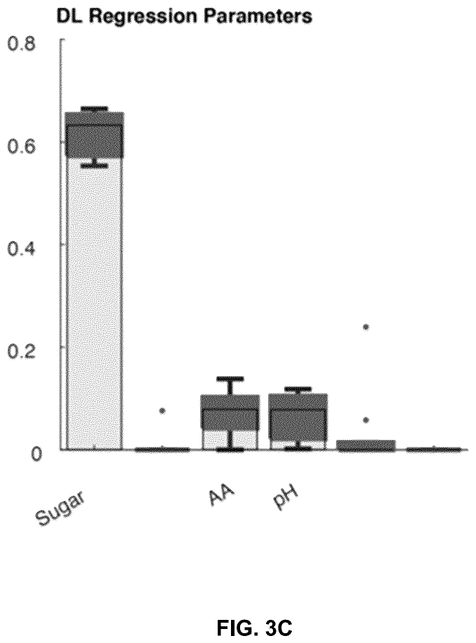

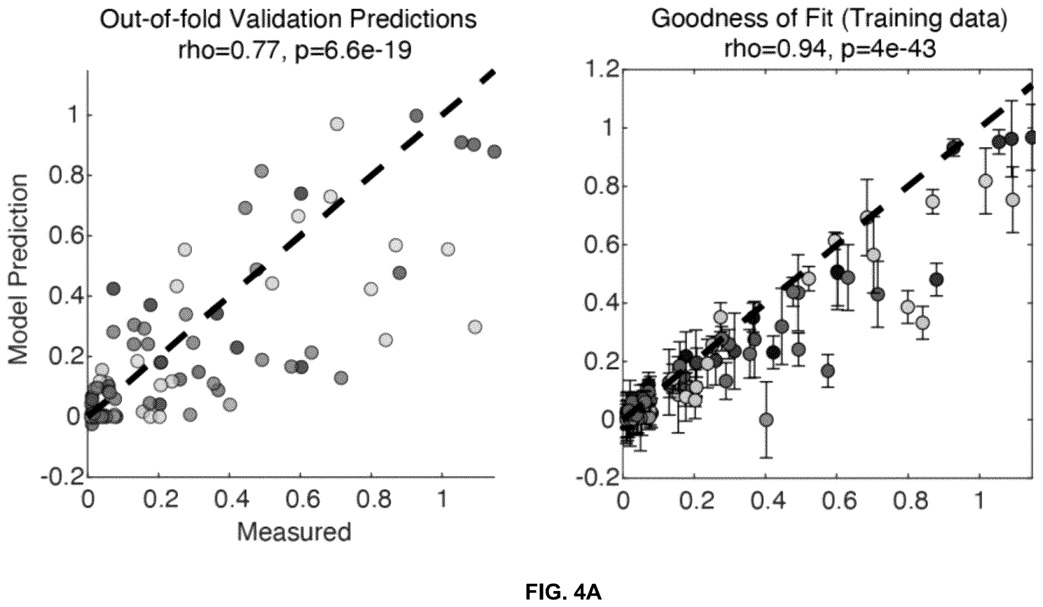

Although we identified a high monoculture-diversity medium in the screening experiment (FIG. 1A, panel c, medium 4), we used model-guided optimization for further improvement. We fit linear regression models with quadratic and interaction terms (multivariate polynomial regression) to predict the carrying capacity of each species from the concentrations of the media component variables (FIG. 1B, equation 6). The media regression model parameters provide an interpretable relationship between the concentration of a given media component and its growth effect on an organism. For instance, the main effects regression parameter corresponding to the “sugars” variable was large for the BL and DL growth models, while all other parameters were small in comparison (FIGS. 1B and 3A-3C). It can be readily observed that these species only grew well in media supplemented with the sugar mixture (FIG. 1A). Interaction parameters in the regression models captured more subtle trends, as these terms indicate a specific combination of independent variables that results in a distinct effect on the measured response. For example, BH had a substantial growth improvement (FIG. 1A, panel b) in media containing both amino acids and sugars (FIG. 1A, panel a). The corresponding interaction parameter (FIGS. 1B and 3A-3C) suggested that the simultaneous presence of these two components enhanced growth more than the sum of their individual contributions alone.

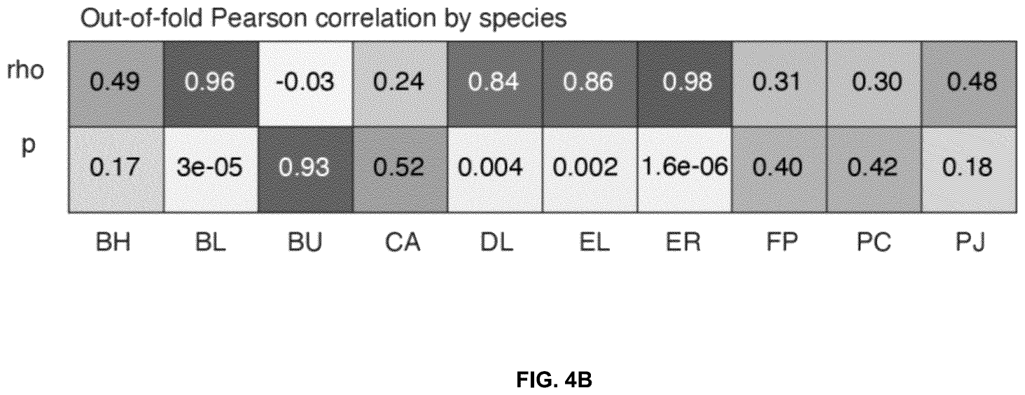

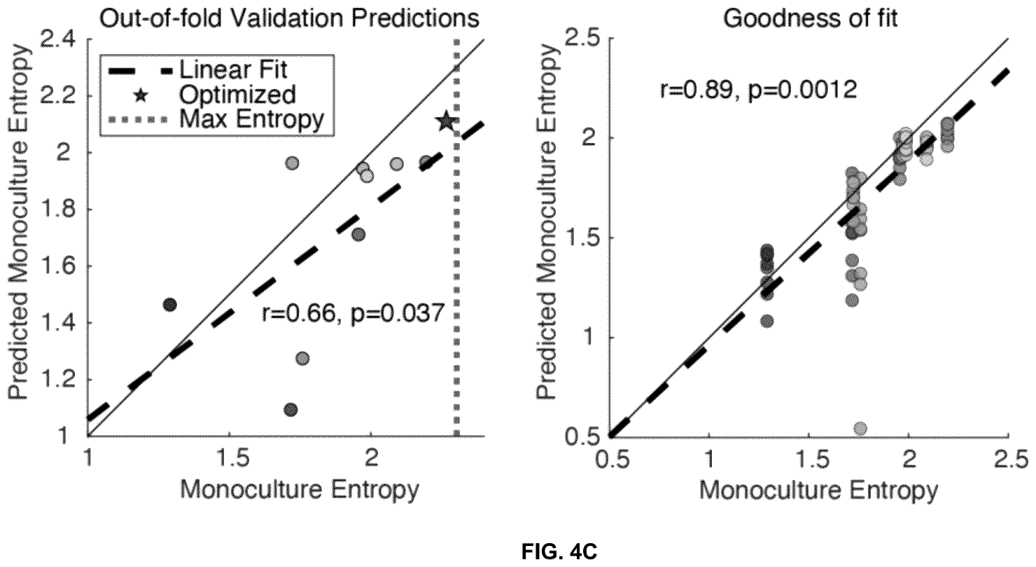

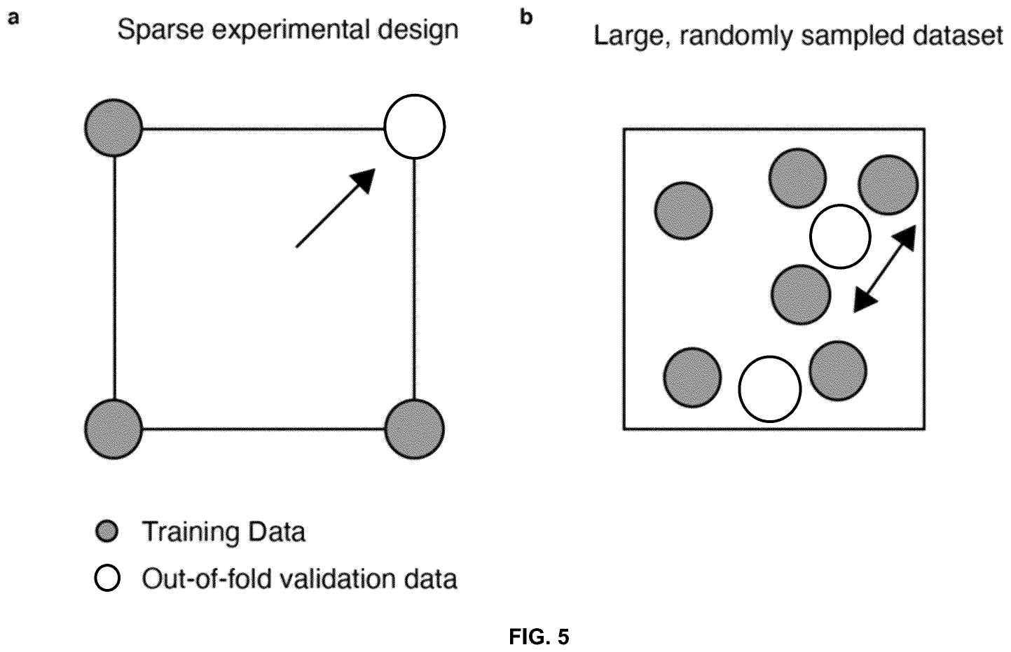

To reduce overfitting and avoid biasing hyperparameters, we implemented elastic net regularization with nested leave-one-out cross validation (Methods). Goodness of fit was high for all species, while validation predictions on out-of-fold data ranged in accuracy (FIGS. 4A-4C). However, we found that the monoculture-diversity calculated from the validation and test predictions of carrying capacity displayed an informative relationship with the measured monoculture-diversity across media conditions (FIG. 1A, Pearson, rho=0.67, 0.034, respectively). Further, we note that such cross validation is not typically used in DoE due to both designed data sparsity and the fact that models are later tested in their ability to predict a new set of inputs that optimizes the system response (FIG. 5). Despite sparse sampling of the design space, the models were predictive of an aggregate property (monoculture-diversity) for new data, even though they were variably predictive of the constituent species.

An optimization procedure (equation 7) was used to identify a profile of media component concentrations that maximized the predicted monoculture-diversity (methods). The predicted concentrations (FIG. 1A, panel d) were similar to medium 4 (FIG. 1A, panel b), but contained 3-fold less sugar. To test this prediction, individual species were grown in the optimized medium. The monoculture-diversity for the optimized medium was close to the maximum possible value, consistent with the model prediction (FIG. 1A, panels e and c). This demonstrates that the model optimization procedure achieved the objective of reducing the variation in individual growth responses while preserving growth of each species.

To determine if monoculture-diversity could inform community Shannon diversity, we cultured the 10-member community from even initial species proportions in the baseline medium 7 (FIG. 1A, panel a), best screened medium 4 (FIG. 1A, panel a), and the optimized medium 10 (FIG. 1A, panel d). The model-guided, monoculture-based optimization process yielded a concomitant improvement in community Shannon diversity (FIG. 1D). Notably, the external pH measured at the endpoint was substantially higher for the community grown in the optimized medium than in medium 4, suggesting that the lower sugar concentration altered community metabolic state (FIG. 1E).

This high-throughput, monoculture-based approach identified a single medium in which all species were capable of similar endpoint growth, and yielded a concomitant increase in the endpoint Shannon diversity of the community. As compared to the baseline medium, Shannon diversity was increased from 53% to 80% of its maximum possible value for a 10 member community. Our approach generalizes to scenarios wherein the desired monoculture-diversity is not identified by an initial experimental screening, as model predictions on simulated media compositions guide optimization of this highly-nonlinear objective.

Predicting Community Composition from Monoculture Growth Kinetics

The initial population density of the constituent members of a microbial community has been shown to impact community assembly. Therefore, we reasoned that we could use a design of experiments approach to further optimize community diversity as a function of inoculum density. However, this constituted a large design space for community experiments, as there are many possible combinations of inoculum proportions for a 10-member community. We first studied the “parts” of our microbial system by characterizing growth kinetics of the monocultures across a wide range of inoculum densities. Monoculture kinetic data were leveraged to build a dynamic community model that was capable of predicting major trends in community assembly as a function of initial population densities (FIG. 6A). This model was used to identify a smaller, more tractable inoculum design space for community experiments in a later stage of this example (FIGS. 9A-9H).

Decreasing inoculum density delayed the time at which a species entered a measurable exponential growth phase (FIG. 6B). BH, ER, and FP tended not to grow (or displayed inconsistent growth between replicates) at lower inoculum densities. The remaining species displayed consistent growth kinetics at most inoculum densities, which spanned several orders of magnitude. A logistic model with a single parameter set (growth rate, carrying capacity) was sufficient to generally represent of a species' growth kinetics across this large range of inoculum densities (FIG. 6B).

We also cultured the full 10-member community from an even inoculum proportion and found that total community growth (optical density of the mixed culture) was substantially less than the sum of the monoculture carrying capacities (FIG. 6C). This observation indicated that negative inter-species interactions dominated the ecological network of the community. Such interactions can arise from competition for limited resources and/or accumulation of inhibitory waste products, especially in a batch culture (a closed system). Supporting this notion, the total growth of microbial communities in batch culture was shown to be a saturating function of the number of species in the community, and plateaued to a relatively constant value across many different 10 to 25-member communities²⁴. Therefore, we reasoned that enforcing an upper limit on total community growth (independent of species composition) could serve as a useful null-hypothesis governing community assembly given unknown, but largely negative, inter-species interactions. Further, we reasoned that competitive fitness should be strongly related to monoculture growth kinetics (i.e. species with faster monoculture growth rates and larger carrying capacities would likely account for a larger fraction of the total growth in the community).

We captured these behaviors in a mathematical model, which we call a “constrained system of logistic equations” (CSLE), and showed that the model's form can be derived from a mass-action type system (Table 1). In this community model, a species grows according to its monoculture logistic kinetics until total community growth is constrained by a “community carrying capacity” (K_{comm}). Thus, a species may cease to grow (dxᵢ/dt→0, the arrow reads as “approaches”) either when its population size approaches its monoculture logistic carrying capacity (xᵢ(t)→Kᵢ) or when total community growth approaches the community carrying capacity (xᵢ(t)→K_{comm}). The CSLE model approximates a “saturated” upper limit on total growth of a large community without defining specific inter-species interactions. We used the measured endpoint optical density of a full 10-member community (of unknown species composition) as the value of K_{comm} (FIG. 6C).

TABLE 1
Description of models.

| Model | Independent | Dependent | Terms/ | Reference | |
|---|---|---|---|---|---|
| Name | Variable(s) | Variable(s) | Parameters | FIGS. | Description |
| Logistic | Single | Monoculture | Mu- | 2A-2G | Fit to timeseries growth |
| models | inoculum | growth over | specific | | measurements for 10 |
| (media | density in | time | exponential | | monocultures on each of nine |
| screening) | multiple media | | growth rate; | | media conditions to determine |
| | | | K-carrying | | kinetic response to media design |
| | | | capacity | | (90 total models). Carrying |
| | | | | | capacity parameter indicates |
| | | | | | steady state growth. |
| Media | Concentrations | Monoculture | Linear and | 1A-1E | Predicts each species' carrying |
| regression | of sugars, | carrying | quadratic | | capacity as a function of media |
| models | amino acids, | capacity | main effects, | | component variables. |
| | yeast extract, | | 2nd and 3rd | | |
| | and pH | | order | | |
| | | | interactions | | |
| Logistic | Range of | Monoculture | Mu- | 6A-6F | Fit to each species' set of |
| models | inoculum | growth over | Specific | | timeseries growth curves across a |
| (inoculum | densities in | time | exponential | | range of inoculum densities. |
| density | optimized | | growth rate | | |
| experiment) | media | | K-carrying | | |
| | | | capacity | | |
| Constrained | Species | Simulated | Mu_i- | 6A-6F | Combines logistic parameters |
| system of | inoculum | community | monoculture | | from above models with a |
| logistic | density in a | growth over | growth rates; | | community level constraint on |
| equations | simulated | time | K_i- | | total growth to account for |
| | community | | monoculture | | negative (likely competitive) |
| | | | carrying | | interactions in batch culture. |
| | | | capacities; | | Estimates community |
| | | | Kcomm- | | composition, trained on |
| | | | community | | monoculture data. Used to |
| | | | level | | estimate a region of inoculum |
| | | | carrying | | design space that is likely to yield |
| | | | capacity | | high Shannon diversity. This |
| | | | | | design space constitutes the |
| | | | | | setpoints of the first DTL cycle. |
| Inoculum | Inoculum | Endpoint | Linear and | 9A-9H | Trained on community data from |
| regression | densities from | species | quadratic | | DTL cycles 1 and 2, predicts |
| models | DTL cycle | growth in the | main effects, | | species abundance in a |
| | 1 + 2 | community | 2^{nd} order | | community from inoculum |
| | | | interactions | | densities of the experimental |
| | | | | | design. Used to design inoculum |
| | | | | | setpoints to maximize endpoint |
| | | | | | diversity for the second and third |
| | | | | | DTL cycles. |
| Generalized | Species | Community | Mu- | 15A-15E | Trained on monoculture kinetic |
| Lotka- | inoculum | growth over | exponential | | data from inoculum density |
| Volterra | densities | time | growth rate, | | experiment (FIGS. 6A-6F), |
| model | | | aij-inter and | | community endpoint data from |
| | | | intra-species | | DTL cycles (FIGS. 9A-9H), and |
| | | | interaction | | several passages of DTL cycles |
| | | | | | (FIG.S 12A-12D). |

The CSLE model, trained only on monoculture kinetic data and the endpoint optical density of a community culture, captured major trends in measured relative species abundances of the community (Pearson correlation, rho=0.71, p=0.021, FIG. 6D). Conversely, modeling community assembly as the sum of independent logistic models (i.e. assuming endpoint community relative abundances are proportional to monoculture carrying capacities) failed to describe community composition (Pearson correlation, rho=0.085, p=0.82, FIG. 6E). This suggests that growth rates and negative interactions are important factors dictating community assembly. The CSLE model correctly predicted that the species with the highest monoculture growth rate (DL, yellow) would occupy a larger fraction of the community than organisms with comparable carrying capacities but much lower growth rates (FIG. 6D).

The CSLE model reaches equilibrium for any community composition in which species' absolute abundances sum to the total growth limit (Σxᵢ(t)=K_{comm}). Thus, in contrast to the logistic model, which has a single positive steady state, the steady state population size of a species in the CSLE model is a continuous function of initial conditions (so long as ΣKᵢ>K_{comm}). This model allowed us to computationally explore how community composition responds to certain inoculum regimes prior to collecting community data (FIG. 6F).

### Tuning Species Inoculum Densities to Optimize Community Shannon Diversity

Microbial community assembly is strongly affected by the initial population sizes of constituent members. We therefore reasoned that adjusting inoculum densities to favor slow growers and delay fast growers would increase endpoint Shannon diversity. A design-test-learn (DTL) cycle was implemented to efficiently search the inoculum design space for setpoints that optimized the endpoint Shannon diversity of the community (FIG. 9A). The iterative DTL approach uses models, trained on community composition data collected throughout previous cycles, to help design a new set of experimental conditions for the subsequent cycle²⁴.

The “design” step comprised constructing various combinations of high, medium, and low levels of inoculum densities for all species. Models were used to guide the assignment of values (setpoints) to the scaled levels of the design. The “test” step used automated liquid handling to array the inocula according to the experimental design (Methods). Community cultures were grown to approximately stationary phase, and species abundances were analyzed (Methods). The “learn” step comprised incorporating the newly collected community abundance data to re-train the models and evaluate predictivity. In cases that model predictivity was insufficient, new design setpoints were systematically assigned based on a qualitative interpretation of growth response (Methods).

The design of the first community inoculum experiment was guided by the CSLE model, which leveraged monoculture kinetics and a total growth constraint to predict community assembly (FIG. 6A). This model captured major trends in community assembly without needing community abundance training data (FIG. 6D). We used optimization to solve for a set of initial conditions that maximized the predicted Shannon diversity (FIG. 6F). This set of initial conditions was used as a central reference point around which the rest of the experimental design was constructed (FIG. 9A). Specifically, we assigned these values to the “center point” of a definitive screening design⁴⁰. The center point of a design refers to a unique condition wherein each independent variable is set at its “medium” level, thus representing the geometric center of the multivariate design space. Throughout the DTL process, we used models to solve for a set of inocula that maximized the predicted diversity, and assigned that set of inocula to the center point condition. The remainder of the experimental designs (combinations of mostly high and low levels) systematically probed various inocula setpoints around the center point. The goal of the designs was both to identify a high diversity condition and collect structured training data to improve model accuracy. The CSLE model was also used to assign the magnitude of the high and low inoculum setpoints, such that the corresponding abundance predictions were normalized to span a 10-fold range for all species (Methods).

Community compositions varied widely as a function of the experimental design conditions (FIG. 9C, DTL 1), confirming that inoculum density was a viable control point for manipulating community assembly. Despite modest monoculture growth kinetics (FIG. 6B), ER overgrew in many conditions (FIG. 9C). We noted that the CSLE model had underpredicted ER in the test community grown from even inoculum proportions (FIG. 6D), likely because the model does not capture positive inter-species interactions, from which this organism appeared to strongly benefit. Since the model underpredicted the abundance of ER, it overpredicted the inoculum proportion of ER needed to achieve high diversity in the first community experiment, resulting in overgrowth.

Regression models with linear, quadratic, and interaction terms were trained to predict the abundance of each species in the community from the inoculum setpoints of the experimental design (Methods). After the first DTL cycle, the inoculum regression models were predictive for half of the species (Pearson correlation, rho>0.7, p-value<1e−6, FIGS. 10A-10B). However, we note that three of the species with accurate models displayed low overall growth (average relative abundance less than 2.5% across design conditions). As such, these models were not practically useful, since predicting maximum diversity (i.e. 10% relative abundance) would result in significant extrapolation.

In the second cycle, for species with inaccurate models or low overall growth, the new center point value was set according to a qualitative interpretation of the growth response. If a species tended to overgrow (ER), the new center point was set at the previous cycle's low level, while if a species tended to undergrow (BL, CA, DL, EL, PC, PJ), its new center point value was set at the previous high value (FIG. 11) (Methods). For species with accurate models that did not display low overall growth, we performed model-guided optimization of the inoculum setpoints to maximize predicted Shannon diversity.

These optimized setpoints were used as the center point values for DTL cycle 2 (Methods). Most species were well-represented in the second experiment, and overgrowth of ER was mitigated (FIG. 9C, “DTL 2”). Inoculum regression models were re-trained on community composition data from both DTL cycles 1 and 2, and predictions were accurate for all species except BL (FIG. 9D) (Pearson correlations greater than 0.70, p-values below 1e−8, FIGS. 10A-10B). Median community diversity was substantially improved over cycle 1 (FIG. 9E).

To identify a high Shannon diversity condition in DTL 3, we repeated the optimization procedure to predict a new center point, using the nine accurate regression models. As we were confident in our model-guided prediction, the high and low levels were set to probe a smaller design space than previous cycles. Since BL consistently undergrew and was poorly predicted by the model, its inoculum density was set to a maximum designated value (Methods). Despite having the largest inoculum and high monoculture fitness, BL remained at low abundance in DTL 3 (FIG. 9C), suggesting it was subjected to strong negative ecological interactions in the community. Notably, the beneficial species FP was well represented in the DTL 3 communities, whereas it was at very low abundance in the even inoculum condition (FIG. 1D)⁴¹⁻⁴³ Overall, the highest Shannon diversity condition was identified in DTL 3, representing 91% of the maximum possible value for a 10-member community (FIG. 9E). This was a substantial improvement from the already high 80% of maximum achieved by medium optimization alone (FIG. 9E).

The set of inoculum densities that yielded the highest Shannon diversity in DTL 3 was correlated to the CLSE optimized inoculum prediction (Pearson correlation between logarithm of inoculum sets, rho=0.66, p=0.039, FIG. 9G). Further, for half of the species, the inoculum density setpoints of the highest diversity condition were within three-fold of the CSLE predicted values (FIG. 9D). These data show that monoculture growth kinetics and a total growth constraint provided sufficient information to predict a useful starting point for the DTL cycle, thereby substantially narrowing the design space that needed to be explored to identify a highly diverse community.

### Robustness, Scale-Up, and Viability

Utility of our process in a manufacturing setting would involve (1) community composition being robust to small variations in inoculum densities that may occur because of technical variability, (2) the microplate process being readily translatable to larger, production-scale equipment, and (3) the endpoint culture containing viable organisms. The conditions of DTL 3 included two-fold inoculum perturbations above and below the center point prediction (i.e. inoculum of each species varied 4-fold across the design). Despite these substantial inoculum perturbations, the coefficient of variation of the endpoint Shannon diversity across design conditions was less than 6% (FIG. 9E). This demonstrated that our process should be quite robust to technical variation. Scale up from 200 μL to 100 mL batch cultures was performed on a sample condition in parallel with DTL 3. Community compositions were similar across this 500-fold difference in scale, suggesting that our process should rapidly translate to production volumes (FIG. 9H, Pearson correlation, rho=0.96, p=8e−6). To evaluate the viability of species in the endpoint community cultures, we passaged (subcultured) a small aliquot of the endpoint community cultures into fresh media, and grew them to approximately stationary phase (Methods). All species were detected in most conditions of the passage, suggesting that the organisms were viable in the original culture (FIGS. 12A-12D).

### Model-Guided Design of Microbial Community Dynamics

Inter-species interactions are major determinants of microbial community assembly^{24,44}. In accordance, we found that the logistic model, which neglects inter-species interactions, was poorly predictive of community assembly from monoculture kinetics (FIG. 6E). By contrast, our CSLE model, which applies a constraint on total growth, predicted a significant degree of community assembly from monoculture (FIG. 6D). To better understand the contributions of inter-species interactions to community assembly, we leveraged the datasets collected during inoculum optimization to train a dynamic computational model. The generalized Lotka-Volterra (gLV) model (equation 13) is a coupled ordinary differential equation model that captures species' community growth dynamics as basal growth rates that are continuously modified by intra and inter-species interactions. This model has successfully predicted complex community dynamics in a number of previous studies, and its interpretable parameter network has been shown to reveal significant inter-species interactions^{24,28,44} We trained a gLV model on our monoculture data (FIG. 6B), community measurements (FIGS. 9C-9E), and a new dataset comprising several “passages” of the community experiments (FIGS. 12A-12D). In passaging experiments, communities were cultured until stationary phase (24-28 hours), aliquots of the communities were transferred into fresh media, and cultured again to stationary phase. This cycle can be repeated indefinitely, allowing the study of community dynamics over longer timescales than a single batch culture, while maintaining the parallelized throughput of the microplate format (Methods)^{1644,45}.

To minimize overfitting of model parameters to the data, we implemented L1 regularization with cross validation (Methods, FIG. 13A). The gLV model was predictive of randomly withheld training data (Pearson correlation, rho=0.91, p=3e−83, FIG. 13B). In the inferred parameter set, BH displayed a large positive effect on ER (FIGS. 13C and 13D). This result is consistent with the underprediction of ER by the CSLE model, in which species interact only via competition (FIG. 6D). The overgrowth of ER in DTL 1 (FIG. 9C) is likely a result of the high inoculum densities of ER and BH in comparison to the relatively low inoculum densities of several species (BU, CA, DL, PC) with which ER competes (mutual negative interactions of magnitude greater than 0.25) (FIG. 13A). BL received negative interactions from all species excluding PJ, and these interactions summed to the largest magnitude of total negative interactions received among all species (FIG. 13D). This suggests that the persistent low community abundance of this organism, despite its robust monoculture growth and high inoculum densities, can be explained as the aggregate effect of many negative interactions in the community.

To test whether our dynamic model could guide the design of sub-communities (three-to-nine-member combinations of our 10 original species) with distinct temporal characteristics, we aimed to design low and high variability of species composition over the timescale of four passages. Communities with low temporal variability are likely needed for dynamic bioprocess strategies, such as fed-batch or continuous cultures, which are commonly used to improve production efficiency⁴⁶⁻⁴⁸. Temporal variability was defined as the sum of the Euclidean distances of the relative species abundance of adjacent passages (e.g. 1-2, 2-3, and 3-4, equation 14). To design sub-communities with low temporal variability, we defined an objective function that considered high ratios of the Shannon diversity to the Euclidean distance across passages (equation 15). The objective function is maximized for an even composition of the full 10-member community which remains constant over four passages. Conversely, it would take on small values for low diversities and/or compositions that vary substantially over the simulated passages. We used optimization techniques to maximize this objective as a function of the initial conditions for all possible (967 total) 3-9 member sub-communities. Notably, among the 967 solutions, we found only 33 unique endpoint species compositions (within a small numerical tolerance) (FIGS. 14A-C). We selected nine of the higher diversity unique solutions for experimental validation, ensuring that each of the ten species were present in at least one of the selected communities. To determine if the model could distinguish between low and high temporal variability, we included three representative communities with predicted high temporal variability (Methods).

The communities designed for low temporal variability had significantly lower Euclidean distances between passages than communities designed for high temporal variability, indicating that their species composition was more constant in time (p-value 8e−6) (FIG. 15B). The model predicted several qualitative characteristics of the high temporal variability communities (FIG. 15C), including the dominant species (highest relative abundance in the final passage). The model also forecasted that FP would be outcompeted when it was a constituent member of high-temporal variability communities (greater than 10-fold lower relative abundance in final passage than initial passage) (FIG. 15C). Notably, the model also identified a low temporal variability subcommunity (FIG. 15D, “BH-EL-FP”) in which FP persisted at a constant abundance over passages two through four. Similarly, BL persisted at a constant abundance across the last three passages when cultured with BU and CA (FIG. 15D, “BU-BL-CA”), despite its low abundance in the first passage (FIGS. 9C-9E) and high abundance in later passages of training data (FIGS. 12A-12D). The model predicted four sub-communities in which at least three species persisted at relatively constant abundance for at least three passages (“BH-EL-FP, BL-BU-CA, CA-EL-ER-PJ, and BU-CA-EL”). These data demonstrate that the gLV model can guide the design of communities that exploit inter-species interactions to support the persistence of low competitive fitness species, as well as mitigate overgrowth of high competitive fitness species. The gLV model trained on monoculture and 10-member community data was moderately predictive of the absolute species abundance in the designed 2-4 member communities (Pearson correlation, rho=0.56, p=9.6e−10, FIG. 16). The unexplained variance in the dataset could be attributed to large differences in species richness in the training (10-species) and test data (2-4 species)²⁴. In sum, the gLV model, informed by variation in inoculum densities of constituent community members, was useful in the prediction and interpretation of community assembly, as well as design of sub-communities with low variability of species composition over time.

### DISCUSSION

We demonstrate that, despite their complexity, microbial community cultures are engineerable systems that respond predictably to multivariate control inputs, including media formulation and inoculum densities. We use model guided approaches for tuning these control inputs to optimize the endpoint Shannon diversity of a synthetic human gut community from 53% to 91% of its maximum possible value (FIG. 9F). Our design of experiments and ecological modeling approaches map control inputs to system response without the need for detailed biological mechanisms. As such, the workflow should be applicable to most any community culture. Our designed set of media and inoculum conditions yields similar community composition at 500-fold higher volumes, suggesting that bench scale process development should translate seamlessly to production scale. Overall, this efficient, scalable blueprint for designing community assembly should help to alleviate the production bottleneck that limits manufacturing of therapeutic communities at clinical, non-profit, and commercial scales. Beyond therapeutic community production, our method should be broadly useful in microbial community bioprocessing. For example, in co-culture metabolic engineering, wherein an engineered pathway is distributed among multiple organisms to exploit division of labor, our method could be applied to tune strain ratios and optimize metabolite product yields^{48,49}.

Our two-stage approach relies on a deeper understanding of community assembly from monoculture, enabling the optimization of a complex microbial system to be reduced to separate, tractable design objectives. We demonstrate that maximizing monoculture-diversity substantially increases community diversity (FIGS. 1A-1E), and that major trends in community composition can be explained by constraining monoculture kinetic models with an upper limit on total growth (FIGS. 6A-6F). We intentionally make use of high-throughput monoculture experiments to first understand the “parts” of our system. Model guided-prediction of community assembly from these well characterized parts allows efficient design of community experiments, which are laborious in comparison to monoculture experiments (due to the DNA sequencing pipeline required to analyze community composition). This aspect of our approach renders it amenable to larger communities and higher resolution experimental designs, as monoculture experiments are almost entirely automated.

We demonstrate that low temporal variability communities can be designed using a dynamic computational model (generalized Lotka-Volterra). Such design capabilities could enable dynamic community bioprocesses (e.g. fed batch or continuous cultures) for improvement of production yield and efficiency. We note that our implementation of the gLV model captures batch culture community growth with flexible, non-equilibrium trajectories. Endpoint community composition is predicted quite accurately (FIG. 13B) as a continuous function of initial conditions, contrary to most ecological implementations of this model, in which many different initial conditions evolve towards a discrete steady-state(s) endpoint. This nuance illustrates that the gLV model can be a practical framework for designing specific community compositions as a function of initial conditions in bioprocessing applications, though it should not be noted that predictions beyond the timeframe of training data collection are likely to be inaccurate. In other words, the model's scalar interaction parameters should be considered a first order Taylor expansion of an unknown higher order function, which, rather than evaluated near an equilibrium point of an open system, as is implied in most theoretical ecological studies, is understood to be evaluated at the endpoint of a batch culture (a closed system)⁵⁰⁻⁵². This dynamic model can be implemented to design time-dependent inoculations and/or net positive inter-species interactions to benefit low fitness organisms. Eventually, dynamic models could be extended to describe resources as well as species, which may enable the design of stable communities as a function of both resources and ecological interactions.

Defined microbial communities hold significant promise for many applications including agriculture, biofuels, and medicine⁵³. Our work contributes to fundamental knowledge of control principles for complex microbial ecosystems, and applies these principles to address the manufacturing bottleneck of therapeutic community production. Eventually, these principles may even be extended to help design strategies for modulating an unhealthy patient's microbiome towards a beneficial state. Diet is well documented to shape gut microbiome composition, while it was recently shown that dosage strength (i.e. inoculum density) was a critical factor in the successful redesign of the first phase three clinical trial of a live microbial therapeutic^{26,54,55}. Overall, initial conditions, environmental resources, and inter-species interactions are likely to be key design parameters for engineering the complex dynamics of microbial community growth and succession, from therapeutic community manufacturing to ecological restoration of a dysbiotic gut microbiome.

### Methods

### Strain Maintenance, Precultures, and Growth Media

The following methods are adapted Hromada 2021, Clark 2021 and Venturelli 2019^{24,27,56}. All anaerobic culturing was carried out in a custom anaerobic chamber (Coy Laboratory Products, Inc) with an atmosphere of 2.5±0.5% H₂, 15±1% CO₂ and balance N₂. All prepared media, stock solutions, and materials were placed in the chamber at least overnight before use to equilibrate with the chamber atmosphere. The strains used in this work were obtained from the sources listed in Table 2 and permanent stocks of each were stored in 25% glycerol at −80° C. Batches of single-use glycerol stocks were produced for each strain by first growing a culture from the permanent stock in anaerobic basal broth (ABB) media (HiMedia or Oxoid) to stationary phase, mixing the culture in an equal volume of 50% glycerol, and aliquoting 400 μL into Matrix Tubes (ThermoFisher) for storage at −80° C. Quality control for each batch of single-use glycerol stocks included (1) plating a sample of the aliquoted mixture onto LB media (Sigma-Aldrich) for incubation at 37° C. in ambient air to detect aerobic contaminants and (2) Illumina sequencing of 16S rDNA isolated from pellets of the aliquoted mixture to verify the identity of the organism. For each experiment, precultures of each species were prepared by thawing a single-use glycerol stock and combining the inoculation volume and media listed in Table 2 to a total volume of 5 mL for stationary incubation at 37° C. Incubation times are also listed in Table 2. Prior to inoculating starter cultures, the workspace and pipettes were cleaned with Spor-klenz, and again with ethanol between strain inoculations. A clean Kim-wipe was held above the workspace to check for “air currents” from equipment fans that could lead to cross contaminations, and equipment was turned off or rearranged as needed. Anaerobic work is conducted in a spatially linear workflow from cleanest to least clean materials (e.g.) tips, clean reagents, cell containing media, then trash, as ordered from dominant to non-dominant hand. Motions above open, sterile containers is restricted to minimum necessary actions.

TABLE 2
Strain information.

| | | | | Inoculum | | |
|---|---|---|---|---|---|---|
| | | | | volume from | Pre- | Pre- |
| Abbre- | | | | glycerol stock | Culture | Culture |
| viation | Species | Strain | Source | (μL) | Media | Time (hr) |
| BH | Blautia | Blautia | DSMZ | 400 | OM | 24 |
| | hydrogenotrophica | hydrogenotrophica | | | | |
| | | S5a33 [DSM | | | | |
| | | 10507] | | | | |
| BL | Bifidobacterium | Bifidobacterium | DSMZ | 400 | 1:1 | 40 |
| | longum subs. | longum subs. | | | OM:ABB | |
| | infantis | Infantis S12 [DSM | | | | |
| | | 20088] | | | | |
| BU | Bacteroides | Bacteroides | DSMZ | 150 | OM | 24 |
| | uniformis | uniformis VPI 0061 | | | | |
| | | [DSM 6597] | | | | |
| CA | Collinsella | Collinsella | DSMZ | 400 | OM | 40 |
| | aerofaciens | aerofaciens VPI | | | | |
| | | 1003 [DSM 3979] | | | | |
| DL | Dorea longicatena | Dorea longicatena | Rey | 150 | 1:1 | 24 |
| | | 111-35 | Lab | | OM:ABB | |
| EL | Eggerthella lenta | Eggerthella lenta | DSMZ | 400 | 1:1 | 24 |
| | | 1899 b [DSM | | | OM:ABB | |
| | | 2243] | | | | |
| ER | Eubacterium | Eubacterium | ATCC | 400 | 1:1 | 40 |
| | rectale | rectale VPI 0990 | | | OM:ABB | |
| | | [ATCC 33656] | | | | |
| FP | Faecalibacterium | Faecalibacterium | DSMZ | 400 | OM | 40 |
| | prausnitzii | prasnitzii A2-165 | | | | |
| | | [DSM 17677] | | | | |
| PC | Prevotella copri | Prevotella copri | DSMZ | 400 | 1:1 | 24 |
| | | CB7 [DSM 18205] | | | OM:ABB | |
| PJ | Parabacteroides | Parabacteroides | DSMZ | 150 | ABB | 24 |
| | johnsonii | johnsonii M-165 | | | | |
| | | [DSM 18315] | | | | |

Abb—5 mL Anaerobic Basal Broth (Oxoid)
OM—5 mL optimized medium at pH 6.7
1:1 OM:ABB—2.5 mL of OM + 2.5 mL of ABB

### Genomic DNA Extraction, DNA Library Preparation, Sequencing, Primer Design, and Data Analysis

DNA extraction, library preparation, and sequencing were performed according to methods described in Hromada 2021 and Clark 2021^{24,16} In brief, cell pellets from about 150 μL of culture were stored at −80° C. following experiments. Genomic DNA was extracted using a 96-well plate adaption of the Qiagen DNeasy protocol. Genomic DNA was normalized to 1 ng/L in molecular grade water, and stored at −20° C. Dual-indexed primers for multiplexed amplicon sequencing of the v3-v4 region of the 16S gene were designed as described previously, and arrayed in 96-well plates using an acoustic liquid handling robot (Echo LabCyte). Genomic DNA and PCR master mix were added to primer plates and amplified prior to sequencing on an Illumina MiSeq platform.

Sequencing data were analyzed as described in Hromada 2021⁵⁶. In brief, basespace Sequencing Hub's FastQ Generation demultiplexed the indices and generated FastQ files. Paired reads were merged using PEAR (Paired-End reAd mergeR) v0.9.0 (Zhang et al, 2014)⁵⁷. Reads were mapped to a reference database of species used in this example, using the mothur vl.40.5, and the Wang method (Wang et al, 2007; Schloss et al, 2009)^{58,59}. Relative abundance was calculated by dividing the read counts mapped to each organism by the total reads in the sample. Absolute abundance was calculated by multiplying the relative abundance of an organism by the OD600 of the sample. Samples were excluded from further analysis if >1% of the reads were assigned to a species not expected to be in the community (indicating contamination).

### Monoculture Media Screening Experiment

The media screening experiment was designed to improve monoculture-diversity (equation 4) on DM38, a chemically defined medium developed, and referenced as the “baseline” medium in the text. Table 3 contains the medium and stock solution recipes referenced in this section. A four-factor, two-level half factorial screening design with appended center point condition was constructed in JMP 15 (SAS institute). “High” absolute design levels for sugar mixture, amino acid mixture, and pH variables (these are key components in DM38) were set at their respective DM38 concentrations. Yeast extract (sterile filtered, not autoclaved) was included to support monoculture growth of F. prausntitzii, as keenly observed by D'Hoe et al³⁸. “Low” design levels were set at 0 g/L for sugars, amino acids, and yeast extract, and 5.7 for pH (according to generally reported ranges for the human large intestine⁶⁰). Stock solutions of sugars, amino acid mixture, and yeast extract were prepared at 20× v/v of their target “high” concentrations, and sterile filtered. The nine media were arrayed according to the experimental design in 2 mL deep-well blocks, using a Tecan Evo liquid handling robot to aliquot the appropriate volume of 20× stocks into 1.4× base medium. The final concentration was brought to 1× using sterile water. The deep well blocks, containing ten sets of the media experimental design, were inoculated from the ten precultures to a 600 nm optical density value of 0.01 (200 μL of sample read Tecan F200 plate reader). Inoculation volumes are calculated as Volume_{(inoc)}=Volume_{(well)}*0.01 OD/(Preculture OD). Inoculation was performed from a sterile trough with a multichannel pipette. Four 200 μL replicates were mixed and aliquoted to sterile, clear bottomed microplates, covered with a transparent seal (Breath EZ), and incubated at 37° C. in Tecan Evo robot. Automated OD600 measurements were recorded every two hours for about 60 hours with a Tecan F200 plate reader.

TABLE 3
Defined media composition

| | Optimized | | Baseline | |
|---|---|---|---|---|
| | Medium | Preculture | Medium | |
| | Concentration | Medium | Concentration | |
| Component | (mM) | Concentration | (mM) | Stock Solution |
| L-alanine | 5.3 | 5.3 | 5.3 | 5x defined amino acid solution |
| | | | | (tyrosine added separately) |
| L-arginine | 22 | 22 | 22 | 5x defined amino acid solution |
| | | | | (tyrosine added separately) |
| L-asparagine | 2.6 | 2.6 | 2.6 | 5x defined amino acid solution |
| | | | | (tyrosine added separately) |
| L-Aspartic | 0.40 | 0.40 | 0.40 | 5x defined amino acid solution |
| acid | | | | (tyrosine added separately) |
| L-Cysteine | 8.4 | 8.4 | 8.4 | 5x defined amino acid solution |
| | | | | (tyrosine added separately) |
| L-Glutamic | 0.66 | 0.66 | 0.66 | 5x defined amino acid solution |
| acid Sodium | | | | (tyrosine added separately) |
| L-glutamine | 2.7 | 2.7 | 2.7 | 5x defined amino acid solution |
| | | | | (tyrosine added separately) |
| L-Glycine | 4.7 | 4.7 | 4.7 | 5x defined amino acid solution |
| | | | | (tyrosine added separately) |
| L-histidine | 1.0 | 1.0 | 1.0 | 5x defined amino acid solution |
| | | | | (tyrosine added separately) |
| L-isoleucine | 1.6 | 1.6 | 1.6 | 5x defined amino acid solution |
| | | | | (tyrosine added separately) |
| L-leucine | 3.6 | 3.6 | 3.6 | 5x defined amino acid solution |
| | | | | (tyrosine added separately) |
| L-lysine | 2.4 | 2.4 | 2.4 | 5x defined amino acid solution |
| | | | | (tyrosine added separately) |
| L- | 0.84 | 0.84 | 0.84 | 5x defined amino acid solution |
| methionine | | | | (tyrosine added separately) |
| L- | 4.5 | 4.5 | 4.5 | 5x defined amino acid solution |
| phenylalanine | | | | (tyrosine added separately) |
| L-proline | 5.9 | 5.9 | 5.9 | 5x defined amino acid solution |
| | | | | (tyrosine added separately) |
| L-serine | 6.4 | 6.4 | 6.4 | 5x defined amino acid solution |
| | | | | (tyrosine added separately) |
| L-threonine | 1.9 | 1.9 | 1.9 | 5x defined amino acid solution |
| | | | | (tyrosine added separately) |
| L-tryptophan | 0.73 | 0.73 | 0.73 | 5x defined amino acid solution |
| | | | | (tyrosine added separately) |
| L-valine | 2.8 | 2.8 | 2.8 | 5x defined amino acid solution |
| | | | | (tyrosine added separately) |
| L-Arabinose | 2.1 | 2.1 | 2.1 | Powder |
| D-Glucose | 6.3 | 6.3 | 6.3 | Powder |
| Maltose | 1.3 | 1.3 | 1.3 | Powder |
| Yeast Extract | 2* | 2* | 0* | Powder |
| (BD bacto) | | | | |
| pH | 5.7 | 6.7 | 6.7 | Titrate with HCL or NaOH prior to |
| | | | | sterile filtering. |
| CuSO4 | 0.021 | 0.021 | 0.021 | 1000x Stock Solution of CaCl₂, |
| | | | | CuSO₄, Pyrixodal, Thymidine |
| Pyrixodal | 9.8E−03 | 9.8E−03 | 9.8E−03 | 1000x Stock Solution of CaCl₂, |
| | | | | CuSO₄, Pyrixodal, Thymidine |
| Thymidine | 0.021 | 0.021 | 0.021 | 1000x Stock Solution of CaCl₂, |
| | | | | CuSO₄, Pyrixodal, Thymidine |
| Xanthine | 0.025 | 0.025 | 0.025 | 1000x Stock Solution of Xanthine |
| | | | | and Folic Acid |
| Folic Acid | 2.4E−03 | 2.4E−03 | 2.4E−03 | 1000x Stock Solution of Xanthine |
| | | | | and Folic Acid |
| Orotic Acid | 0.064 | 0.064 | 0.064 | 100x Stock Solution of Orotic Acid |
| AlK(SO₄)₂ | 3.9E−03 | 3.9E−03 | 3.9E−03 | 100x Stock Solution purchased |
| | | | | from Vendor (ATCC Mineral Mix) |
| Co(NO₃)₂ | 0.055 | 0.055 | 0.055 | 100x Stock Solution purchased |
| | | | | from Vendor (ATCC Mineral Mix) |
| EDTA | 0.17 | 0.17 | 0.17 | 100x Stock Solution purchased |
| | | | | from Vendor (ATCC Mineral Mix) |
| FeSO₄ | 0.066 | 0.066 | 0.066 | 100x Stock Solution purchased |
| | | | | from Vendor (ATCC Mineral Mix) |
| H₃BO₃ | 0.016 | 0.016 | 0.016 | 100x Stock Solution purchased |
| | | | | from Vendor (ATCC Mineral Mix) |
| MgSO₄ | 2.7 | 2.7 | 2.7 | 100x Stock Solution purchased |
| | | | | from Vendor (ATCC Mineral Mix) |
| MnSO₄ | 0.33 | 0.33 | 0.33 | 100x Stock Solution purchased |
| | | | | from Vendor (ATCC Mineral Mix) |
| Na₂MoO₄ | 4.9E−03 | 4.9E−03 | 4.9E−03 | 100x Stock Solution purchased |
| | | | | from Vendor (ATCC Mineral Mix) |
| Na₂SeO₃ | 5.8E−04 | 5.8E−04 | 5.8E−04 | 100x Stock Solution purchased |
| | | | | from Vendor (ATCC Mineral Mix) |
| Na₂WO₄ | 3.4E−03 | 3.4E−03 | 3.4E−03 | 100x Stock Solution purchased |
| | | | | from Vendor (ATCC Mineral Mix) |
| NaCl | 1.7 | 1.7 | 1.7 | 100x Stock Solution purchased |
| | | | | from Vendor (ATCC Mineral Mix) |
| NiCl₂ | 0.015 | 0.015 | 0.015 | 100x Stock Solution purchased |
| | | | | from Vendor (ATCC Mineral Mix) |
| Potassium | 4.5E−04 | 4.5E−04 | 4.5E−04 | 100x Stock Solution purchased |
| Hydroxide | | | | from Vendor (ATCC Mineral Mix) |
| Sodium | 7.0 | 7.0 | 7.0 | 100x Stock Solution purchased |
| Sulfate | | | | from Vendor (ATCC Mineral Mix) |
| ZnSO₄ | 0.062 | 0.062 | 0.062 | 100x Stock Solution purchased |
| | | | | from Vendor (ATCC Mineral Mix) |
| Ca-D- | 4.2E−04 | 4.2E−04 | 4.2E−04 | 100x vitamin mix solution (ANL |
| pantothenate | | | | Vitamin Mix) |
| Cobalamin | 1.5E−06 | 1.5E−06 | 1.5E−06 | 100x vitamin mix solution (ANL |
| (B12) | | | | Vitamin Mix) |
| Pyridoxine | 9.7E−04 | 9.7E−04 | 9.7E−04 | 100x vitamin mix solution (ANL |
| HCl | | | | Vitamin Mix) |
| Riboflavin | 5.3E−04 | 5.3E−04 | 5.3E−04 | 100x vitamin mix solution (ANL |
| | | | | Vitamin Mix) |
| Tetrahydrofolic | 2.8E−06 | 2.8E−06 | 2.8E−06 | 100x vitamin mix solution (ANL |
| Acid | | | | Vitamin Mix) |
| Thiamine | 5.9E−04 | 5.9E−04 | 5.9E−04 | 100x vitamin mix solution (ANL |
| HCl | | | | Vitamin Mix) |
| p- | 0.073 | 0.073 | 0.073 | 250x 4C Stock Solution (contains p- |
| Aminobenzoic | | | | Aminobenzoic Acid and |
| Acid | | | | Pyridoxamine) |
| Pyridoxamine | 0.021 | 0.021 | 0.021 | 250x 4C Stock Solution (contains p- |
| | | | | Aminobenzoic Acid and |
| | | | | Pyridoxamine) |
| Ammonium | 9.3 | 9.3 | 9.3 | 250x RT Stock Solution (contains |
| Chloride | | | | MgCl2, KH2PO4, NH4Cl, |
| | | | | Nicotinamide) |
| Magnesium | 1.7 | 1.7 | 1.7 | 250x RT Stock Solution (contains |
| Chloride | | | | MgCl2, KH2PO4, NH4Cl, |
| | | | | Nicotinamide) |
| Potassium | 6.6 | 6.6 | 6.6 | 250x RT Stock Solution (contains |
| Phosphate | | | | MgC12, KH2PO4, NH4Cl, |
| | | | | Nicotinamide) |
| Nicotinamide | 0.034 | 0.034 | 0.034 | 250x RT Stock Solution (contains |
| | | | | MgCl2, KH2PO4, NH4Cl, |
| | | | | Nicotinamide) |
| Hematin | 0.015 | 0.015 | 0.015 | 250x Stock Solution of Hematin, |
| | | | | solubilized with NaOH |
| Cytosine | 5.9E−05 | 5.9E−05 | 5.9E−05 | 30x Stock Solution purchased from |
| | | | | Vendor (10x ACGU Mix) |
| Guanine | 6.0E−05 | 6.0E−05 | 6.0E−05 | 30x Stock Solution purchased from |
| | | | | Vendor (10x ACGU Mix) |
| L-Adenine | 6.0E−05 | 6.0E−05 | 6.0E−05 | 30x Stock Solution purchased from |
| | | | | Vendor (10x ACGU Mix) |
| Uracil | 5.9E−05 | 5.9E−05 | 5.9E−05 | 30x Stock Solution purchased from |
| | | | | Vendor (10x ACGU Mix) |
| Biotin | 0.041 | 0.041 | 0.041 | 50x Individual Stock Solution |
| | | | | (Stored 4° C.) |
| L-tyrosine | 3.2 | 3.2 | 3.2 | Powder |
| MOPS | 72 | 72 | 72 | Powder |
| Sodium | 48 | 48 | 48 | Powder |
| Bicarbonate | | | | |
| Sodium | 32.9 | 32.9 | 32.9 | Powder |
| Acetate | | | | |
| ABB powder, | 0.354* | 0.354* | 0 | Powder |
| Oxoid | | | | |

*Units of g/L not mM for complex components.

### Modeling Monoculture Growth

Monoculture timeseries growth data was fit with logistic differential equations (equation 3), and the carrying capacity parameter was used as a readout of growth response. Carrying capacity serves as a “smoothed,” time independent maximum growth value. Smoothing is required because raw data may contain outlier values t due to condensation on the transparent plate seal or other mechanical abnormalities. If computational resources or expertise are limited, the growth response could also be taken as the maximum value of a smoothed timeseries (e.g. after applying a running average filter). The baseline of the OD600 timeseries data was computationally “blanked” (e.g. normalized) to the known inoculum density. Each fitting was performed independently using bounded, nonlinear regression with MATLAB's “fmincon” function, which returns the logistic parameter set (μ, K) that minimizes the sum of squared errors between the model predictions and the experimental data. All timeseries were truncated to 30 hours to remove death phases. Outlier detection was performed by comparing the z-score of the mean OD600 across replicates, to omit replicates that did not grow.

Multivariate polynomial regression models were fit to predict each species' carrying capacity parameter (growth response) as a function of the scaled media design matrix (predictors). We offer the clarification that although the model is a multivariate polynomial function of the design variables, the regression problem itself is linear, as the higher order predictors are treated as “new” variables whose value is calculated prior to regression. The polynomial structure (equation 6) contained main effects (X₁), quadratic main effects (X₁²), and both second and third order interaction terms (X₁*X₂ and X₁*X₂*X₃). The double and triple sum terms in this equation represent the upper triangular matrix of unique two-factor interaction parameters and three dimensional upper triangular matrix of third order interaction parameters (e.g. X₁*X₂=X₂*X₁ so only one of these predictor terms should be included). The estimation of quadratic terms is contingent on the inclusion of a center point condition in the otherwise two-level experimental design. Because the models are data limited, elastic net regularization cross validation were performed to reduce overfitting. The elastic net and regularization coefficient hyperparameters were selected using nested leave-one-out cross validation and MATLAB's “lasso” function. For each species, the 9-condition dataset (9×16 predictor matrix and 9×1 growth response vector) is partitioned into all nine possible combinations of eight conditions/rows using MATLAB's “crossvalind” function (first partitioning). The “lasso” function is called with the cross-validation argument, meaning it internally performs a second round of leave-one-out cross validation to identify the regularization and elastic net coefficients (hyperparameters) that minimize the out-of-fold mean sum of squared errors for the “internal” cross validation sets. Only the hyperparameters, but not the regression parameters, are returned at this stage. The Lasso function is then called again without the cross-validation arguments, receiving the previously identified hyperparameters as arguments to find a best fit parameter set for the “first partitioning” of the original dataset. This is performed for each partition of the original dataset, such that each regression model is actually an ensemble model with nine parameter sets, each corresponding to one “leave-one-out” partitioning of the data. Each parameter set has its own, independently identified hyperparameters, such that none of the hyperparameters are biased by training on the entirety of the dataset. The models are validated by making “out-of-fold predictions”, meaning using the parameters trained on each of the nine partitions of eight datapoints to predict the one datapoint that is not contained in that partition. When the models are called to make a new prediction (e.g. for the optimization script), the nine predictions of the “ensemble” are averaged to a scalar value.

### Media Optimization Script

A constrained optimization problem was solved using MATLAB's “fmincon” function to solve for the concentration profile of sugar mixture, amino acid mixture, yeast extract, and pH that maximized the monoculture-diversity (equations 6, 7, and 8). The upper and lower bound arguments to the “fmincon” function are set such to constrain the solution within the original experimental design levels (e.g. sugars between 0 and 9.45 g/L, yeast extract between 0 and 2 g/L, amino acids between 0 and 10.7 g/L, and pH between 5.7 and 6.7). The function is initialized with a guess of the sugars, amino acids, yeast extract, and pH concentrations. The “objective function” references the received concentration inputs and calls the linear regression models to make a prediction of each species' carrying capacity from this set of resource concentrations. From these ten carrying capacity predictions, the predicted monoculture-diversity is calculated. The “fmincon” function then iteratively solves for the single concentration of the resources that maximizes the predicted monoculture diversity, using the default interior point algorithm.

### Monoculture Growth Kinetics Over a Range of Inoculum Densities

Deep well blocks were filled with 1000 μL of the optimized medium. Species were precultured and inoculated into each of the first ten wells of the first row of the block at a density of 0.01 OD600. A multichannel pipet was used to mix and perform six 10-fold volume/volume serial dilutions of the first row down the rows of the plate. Three replicate microtiter plates with 200 μL each well were aliquoted from the deep well block and covered with a transparent seal (Breath EZ). Plates were incubated and timeseries OD600 was recorded as previously described.

The low inoculum densities resulted in growth curves that “appeared” to have a long lag phase, but were much more likely to be undergoing exponential growth far below the limit of detection of the plate reader. Timeseries data from inoculum conditions that did not result in reproducible growth were omitted from the dataset, and data was normalized to a baseline of value of the target inoculum as previously described. The exponential and stationary phase data from each species' set growth curves was isolated as the values greater than the assumed 0.05 lower limit of detection for the plate reader. The true limit of detection of the reader is 0.001, but data below 0.05 has high signal-to-noise ratios for microbial growth. As such, the “measured” initial conditions were omitted from the dataset. Nonlinear regression was used to solve for the single logistic parameter set (μ, K) and the set of initial conditions (one for each growth curve in the set) that minimized the sum of squared errors between the model predictions and the exponential phase data. A vector of two logistic parameters and one-to-six initial conditions (depending on how many dilutions grew reproducibly) was passed as variables to the “fmincon” solver. The objective function then parsed the vector into initial conditions and ODE parameters, then called an ODE solver to generate model predictions. The value of the objective function is the sum of mean squared errors between the model predictions and the exponential phase data for all growth curves in the set. The “fmincon” function returns the vector of parameters and initial conditions that minimize the objective function. The computationally fitted initial conditions were plotted in log-log space against the experimental initial conditions, and a first order linear regression was performed to map the log transformed experimental initial conditions to the log transformed, computationally fitted initial conditions, using sets of values that fell in the linear range.

### Design of the First Community Inoculum Density Experiment

The experimental design chosen for the first inoculum screening was a nine-factor, three-level definitive screening design⁴⁰. These designs have three levels for each variable, improving estimation of the quadratic effects that are likely important for approximating the endpoint of exponential microbial growth with a polynomial function. The scaled design matrix was constructed in JMP 15. Inoculum concentrations were assigned to the scaled experimental design levels using solutions from the constrained system of logistic equations model. The constrained system of logistic equations was simulated in MATLAB, using the growth rate and carrying capacity parameters as fitted to monoculture data (described in the previous section). The community carrying capacity parameter K_{comm} was taken as the maximum OD600 of a full community culture inoculated from an even inoculum (all species inoculated to 0.001 OD600). To find the set of initial conditions that maximized the Shannon diversity of the CSLE model at steady state, a constrained optimization problem was solved with MATLAB's “fmincon” function. The variables optimized by the “fmincon” solver comprised the set of all species' initial conditions. The objective function internally maps these initial conditions to the computational space equivalent (using the linear regression functions previously described), and simulates community growth by calling a CSLE ODE function. The “fmincon” solver iteratively solves for the set of initial conditions that minimized the Shannon diversity (equation 1) of the steady state population abundances using the default interior point algorithm. The initial condition solutions are constrained by lower bounds of the experimental inoculum conditions that did not grow, such that the solver does not return initial condition that are too low to use in practice (an issue that can arise when modeling populations of organisms with continuous numerical variables). The total inoculum is constrained using a linear inequality argument such that the sum of all initial conditions did not exceed 0.02 (F. prausnitzii was fixed at 0.01; the sum of the other nine species was constrained to below 0.01). The high inoculum level for each species was solved for by fixing all other species' initial conditions at the maximum diversity solution (center point), then finding the initial condition for that species which yielded a 3.3-fold higher steady state abundance than the center point condition. Specifically, “fmincon” was called to minimize the squared error between the simulation and 3.3 times the steady state abundance of that species' maximum diversity solution as a function of that species initial condition. This was iteratively performed to find all species' “high” initial condition levels for the experimental design. The low levels were set symmetrically to the “high” levels in log space, (e.g. the center point was multiplied and divided by the same x-fold factor), such that a CSLE simulation of the experimental design conditions predicted maximum diversity at the center point, and an approximate a 10-fold range of steady state abundances of each species occurred between “high” and “low” design levels. This approach accounts for the fact that a species with a very fast exponential growth rate will need a much larger perturbation (in comparison to a species with a low exponential growth rate) to its initial condition to achieve a similar change in the endpoint growth.

### Community Inoculum Density Experiments

Experimental designs were arrayed with a Tecan Evoware liquid handling robot. Before inoculation, precultures were centrifuged at 4000 rpm, 7.5 minutes. Anaerobically, the supernatant was decanted, the pellet was dry-vortexed, and resuspended in fresh optimized medium using a serological pipette. Two 24-well blocks were used to array various densities of the precultures. The top row contained a high-density preculture, the second row contained a mid-density preculture, and the third row contained a low-density preculture. The concentration of the high-density preculture well for each species was calculated by finding the number of ten-fold dilutions of the measured preculture OD which resulted in the smallest inoculation volume greater than 7 μL. In other words, we calculated the lowest volume that can be accurately pipetted by the robot to inoculate the deep well block to its target “high” experimental level. For example, if species A grew to a preculture OD of 0.2 and was to be inoculated to a target “high” level of 0.0001 in a volume of 700 μL, then the high-density preculture well would contain a hundred-fold dilution of the preculture (0.002 OD600), such that “high” experimental condition would be inoculated with V=0.0001 OD*700 μL/(0.002)=35 μL. This strategy was implemented because any volume less than 7 μL could not be pipetted accurately, while larger inoculum volumes would quickly accumulate and result in a scenario where the sum of all species' inoculum volumes exceeds the target culture volume. The “mid” and “low” preculture wells were filled by diluting the “high” preculture well by the same x-fold ratio of the high to center point design levels (and equivalently the ratio between the center point and low levels). Two serial dilutions of this ratio were performed from high to mid, and mid to low preculture wells for each species, such that each species' high, center point, and low design levels were inoculated with a constant volume from the high, mid, and low preculture wells, respectively. A 200 μL aliquot of the inoculated deep well block was transferred to a 200 μL microplate, covered with a breathable seal, and incubated in the plater reader at 37° C. Labware and culture conditions were consistent between monospecies and coculture, as it should be noted that differences in labware geometries, particularly surface to volume ratios, can affect anaerobic microbial growth dynamics. Optical density measurements were recorded at 28+/−1 hour in an F200 plate reader. 150 μL of the endpoint culture was transferred to a sterile 1 mL deep well block and centrifuged at 2400×g for 10 minutes. The supernatant was removed, and the pellet was stored at −80° C. Twenty L of the supernatant was used to measure pH using a spectrophotometric phenol red assay²⁴

### Design of Subsequent Inoculum Experiments

Linear regression was used to fit polynomial models (equation 11) to predict each species' community abundance from the inoculum design matrix, using the nested cross validation approach detailed in the media design methods section. The inoculum design matrix was log 10 transformed to scale the values prior to fitting. The models trained on cycles 1+2 community data were evaluated on withheld test data (5 of 59 total conditions) to demonstrate predictivity of the approach (FIG. 9B). Replicates were averaged prior to fitting to avoid biasing test/validation data with conditions contained in training data. Validation predictions and Pearson correlation coefficients for both cycles' models are shown in supplemental materials. Models that were deemed predictive were used in a multi-objective optimization problem (equation 12, details in following paragraph) to predict an updated center point for the new experimental design. Any desired target composition (not only even endpoint, i.e. maximum diversity) can be designed with this approach by updating this target vector with the desired endpoint abundances. Species whose models were not deemed predictive were adjusted using a rational “frameshift” strategy (FIG. 11). The “frameshift” involves selection of new design level absolute setpoints as follows: if a species overgrew (saturated response) in the previous experiment, the new center point level is set at the previous low level. If a species undergrew (non-measurable or very low growth in comparison to other species), its updated center point inoculum level is set at the previous “high” level. These new center point levels were thus equivalent to the extrema of the previous design space, and could be used as inputs to the regression models (without forcing the models to extrapolate beyond the bounds of training data). We note that the DTL process could probably be carried out using only the “frameshift” strategy to approach a design goal. The magnitude of the levels (x-fold of center point) were maintained between cycles one and two, unless the total range between high and low exceeded two orders of magnitude, in which case it was constrained to two orders of magnitude. In cycle three, the experimental design was modified to a twelve-run Placket-Burman screening with center point, with levels set at two-fold above and below center point. This adjustment of the levels initially informed by the CSLE model (cycle 1 levels) is a qualitative decision that reflects the purpose of the designs. Cycle one had large magnitude levels because it was meant to explore a large design space. Cycle two levels were constrained to two orders of magnitude or less to balance searching the design space with the probability of finding a high diversity condition. Cycle three levels were constrained to only two-fold because the purpose of the design was to demonstrate the robustness of a high confidence prediction to small variations, rather than to explore the design space and gather data for further model training.

A constrained multi-objective optimization problem was solved to minimize the error between target abundances and regression model predictions. This objective function is a more strict definition of maximizing Shannon diversity at a particular total species abundance, and was chosen because maximizing the Shannon diversity can return very low total growth solutions. Additionally, it is also a more flexible approach, as it allows the user to define an exact target community composition. We targeted an even endpoint abundance for each organism of magnitude (average community OD)/(# of species), where the average community OD was the average endpoint OD across all the conditions of the previous experiment.

### Passaging Experiments

A serial subculture is performed by mixing well and diluting 20 μL of the endpoint coculture into 500 μL of fresh medium (25-fold v/v). The new culture is then aliquoted (200 L) into a microplate and incubated as previously described. This process was performed three times for the first inoculum design (DLT cycle 1) and once for DTL cycle 2. The data is available in FIGS. 12A-12D.

### Generalized Lotka-Volterra Model Training and Validation

The parameters of a generalized Lotka-Volterra (gLV) model were fit to monoculture timeseries data and 10-member community initial and endpoint data. The training data additionally included three passages of the first inoculum screening and one subculture of the third, as described in the previous paragraph. The passages were treated as independent experiments with initial conditions calculated from the previous culture's endpoint abundances divided by the 20, corresponding to the volumetric dilution. The gLV model was fit to experimental data using MATLAB's “fmincon” solver to minimize a cost function as by finding the best parameter values. The cost function comprised the sum of squared errors between the model predictions and data, plus an L1 regularization penalty to minimize overfitting, as previously described²⁷. The upper bounds for growth rate terms μᵢ, self interaction terms aᵢᵢ, and interspecies interaction terms a_{ij,i≠j}, were 3, 10, and 0, respectively. The lower bounds for these quantities were 0, −10, and −10, respectively. Self-interaction terms must be non-positive and growth rate terms must be non-negative to avoid divergence and maintain biological meaning. The “MaxFunctionEvaluation” and “MaxIterations” arguments for “fmincon” were both set to “Inf” via the “optimoptions” function to allow the solver sufficient time to converge. The solver was initialized with the monoculture growth rates, monoculture derived self-interaction terms, and zeros as respective initial guesses for the gLV growth rates, gLV self-interaction terms, and gLV interspecies interaction terms. Zero is a logical initial guess for unknown parameters subject to L1 regularization, which pushes poorly constrained parameters towards zero. The community data was randomly partitioned into test and training+validation datasets comprising 10% and 90% of the data, respectively, using MATLAB's “randsample” function. Monoculture data was not included in validation or test sets because it is collected at high-resolution time intervals, thus monoculture parameters are very well constrained. The regularization coefficient was found by scanning a logarithmic range of values and identifying the value that corresponded to the lowest averaged sum of squared errors across out-of-fold predictions (5-fold cross validation, training+validation data partitioned using MATLAB's “crossvalind” function). A best-fit parameter set was then re-fitted to the training+validation dataset using the identified regularization coefficient. The model was evaluated for predictivity on the randomly withheld test data. The parameter value heatmap, histogram and, predicted vs. measured scatter plot are shown in supplemental materials.

### Design of Temporal Variability in Subcommunities

The best-fit gLV model was used to design communities with low temporal variability over the course of four simulated passages. For all 967 possible 3-to-9-member subcommunities (i.e. sum of 10 choose k for k=3 to 9), a constrained optimization problem was solved to minimize an objective function as a function of the initial conditions of the species present in the subcommunity. Species absence in subcommunities were simulated by forcing both upper and lower bounds of the omitted species' population sizes to zero. Initial condition solutions were bounded between zero and 0.01 simulated OD600 for species present in a subcommunity. The endpoint compositions of resulting from all initial condition solutions were sorted into unique results using MATLAB's “uniquetol” function (within a numerical tolerance of 0.05 for each species). Nine of these communities were chosen for experimental validation on the qualitative criteria of having all species present in the set of subcommunities. These nine communities were of size 2−4 members. As a comparison, we designed four-member high temporal variability subcommunities by maximizing the product, rather than the ratio, of the diversity and distance terms in equation 15. The nine low temporal variability subcommunities and three high temporal subcommunities were inoculated at densities according to the computational predictions. These inoculum conditions spanned orders of magnitude with no symmetry between conditions. The following strategy was used to inoculate these conditions: an “inoculum” 96-well 2 mL deep well block was prepared in which each species' preculture material was diluted to 0.1 in row one. Tenfold serial dilutions were then performed such that preculture material was available for pipetting at a range of 0.1 to 10⁻⁵ OD600. The liquid handling robot was assigned to aspirate from whichever well would result in the smallest aspiration volume greater than 7 μL, for each species in each condition. The culture was incubated, passaged, and sampled as previously described.

### Equations

### Shannon Diversity:

$$SD = - {\sum }_{i}^{Species} ( {X}_{fr , i} ) ln ( {X}_{fr , i} ) \qquad ( 1 )$$

X_{fr,i}—fractional abundance of species i in a community

### Fractional Abundance:

$${X}_{fr , i} = \frac{{X}_{i}}{\sum {X}_{j}} \qquad ( 2 )$$

Xᵢ—absolute population size of species i in a community

### Logistic Differential Equation:

$$\frac{{dX}_{i}}{dt} = {μ}_{i} ( 1 - \frac{{X}_{i}}{{K}_{i}} ) {X}_{i} \qquad ( 3 )$$

$$\frac{{dX}_{i}}{dt}$$

—rate of change of population size of species i
μᵢ—specific exponential growth rate of species i
Kᵢ—carrying capacity

### Monoculture-Diversity (Effective Shannon Diversity of Normalized Monoculture Carrying Capacities Corresponding to a Particular Medium):

$${SD}_{mono} = - {\sum }_{i}^{species} ( {K}_{fr , i} ) ln ( {K}_{fr , i} ) \qquad ( 4 )$$

K_{fr,i}—normalized (fractional) carrying capacity of species i

### Normalized (Fractional) Carrying Capacity:

$${K}_{fr , i} = \frac{{K}_{i}}{{\sum }_{j} {K}_{j}} \qquad ( 5 )$$

Kᵢ—Logistic carrying capacity parameter, species i
Σⱼ Kⱼ—Sum of carrying capacity parameters for all species on a particular medium condition
   Linear Regression Model Predicting Carrying Capacity from Resources:

$${\hat{K}}_{i} = {\sum }_{l = 1}^{r} {β}_{l}^{ME} {X}_{l} + {\sum }_{l = 1}^{r} {β}_{l}^{QE} {X}_{l}^{2} + {\sum }_{l = 1}^{r - 1} {\sum }_{m = l + 1}^{r} {β}_{lm}^{IX 2} {X}_{l} * {X}_{m} ⋯ + {\sum }_{l = 1}^{r - 2} {\sum }_{m = l + 1}^{r - 1} {\sum }_{n = m + 1}^{r} {β}_{lmn}^{IX 3} {X}_{l} * {X}_{m} * {X}_{n} \qquad ( 6 )$$

\hat{K}ᵢ— predicted carrying capacity of species i
βₗ^{ME}—main effects parameters
Xₗ—main effects parameters (independent variable, e.g. media component concentration)
r—number of media variables, r=4
βₗ^{QE}—quadratic effects parameters
Xₗ²—quadratic main effects predictors
Xₗₘ^{lX2}—Interaction parameters, 2nd order
Xₗₘ^{lX3}—Interaction parameters, 3rd order

### Objective Function for Media Optimization:

$$maximize ( {mono}_{} = - {\sum }_{i}^{species} ( {\hat{K}}_{fr , i} ) ln ( {\hat{K}}_{fr , i} ) ) \qquad ( 7 )$$

[symbol] ₘₒₙₒ—predicted monoculture-diversity

### Predicted Fractional Carrying Capacity:

$${\hat{K}}_{fr , i} = \frac{{\hat{K}}_{i}}{{\sum }_{j} {\hat{K}}_{j}} \qquad ( 8 )$$

### Constrained System of Logistic Equations

$${\hat{X}}_{F , i} = {\int }_{t 0}^{tF} {μ}_{i} ( 1 - \frac{{X}_{i}}{{K}_{i}} ) ( 1 - \frac{\sum {X}_{j}}{{K}_{comm}} ) {X}_{i} dt \qquad ( 9 )$$

\hat{X}_{F,i}—predicted Final (endpoint) abundance of species i in a simulated community
ΣXⱼ—Total community growth
K_{comm}—community carrying capacity
   Objective Function for Maximizing Shannon Diversity of CSLE prediction:

$$maximize ( \hat{D} = - {\sum }_{i}^{species} ( {\hat{X}}_{F , fr , i} ) ln ( {\hat{X}}_{F , fr , i} ) ) \qquad ( 10 )$$

\hat{X}_{F,f,r,i}—Final fractional abundance of species i in CSLE simulation

### Linear Regression Model Predicting Final Community Abundance From Inoculum Densities

$${\hat{X}}_{F , i} = {\sum }_{l = 1}^{s} {β}_{l}^{ME} {X}_{0 , l} + {\sum }_{l = 1}^{s} {β}_{l}^{QE} {X}_{0 , l}^{2} + {\sum }_{l = 1}^{s - 1} {\sum }_{m = l + 1}^{s} {β}_{lm}^{IX} {X}_{0 , l} * {X}_{0 , m} \qquad ( 11 )$$

\hat{X}_{F,i}—predicted final coculture abundance, species i
s=10 (number of species)
remaining terms as defined in 6

### Objective Function Design of New Inoculum Experiment Center Points:

$$minimize ( {\sum }_{i = 1}^{species} {( {X}_{TARG , i} - {\hat{X}}_{F , i} )}^{2} ) \qquad ( 12 )$$

X_{T ARG,i}—target (desired) endpoint abundance of species i

### Generalized Lotka-Volterra Model:

$$\frac{{dx}_{i}}{dt} = ( {μ}_{i} + {\sum }_{j = 1}^{n} {a}_{ij} {x}_{j} ) {x}_{i} \qquad ( 13 )$$

xᵢ—“i'th” species in an “n” member community
μᵢ—exponential growth rate parameter, species i
aᵢᵢ—self interaction parameter, a transformation of the logistic carrying capacity,

$${a}_{ii} = - {μ}_{i} / {K}_{i}$$

a_{ij,i≠j}—inter-species interaction parameters
   In the single species case, the gLV model reduces to the logistic equation. In the n species case with no inter-species interactions, the gLV model reduces to n logistic equations.

### Euclidean Distance (of Adjacent Passages):

$${Eu}_{P} ( X ) = {( {\sum }_{i}^{n} {( {X}_{p , i} - {X}_{( p - 1 ) , i} )}^{2} )}^{1 / 2} \qquad ( 14 )$$

X_{p,i}—fractional abundance of species “i” at the endpoint of passage “p”

### Objective Function for Low Temporal Variability Communities:

$$maximize ( \frac{{\sum }_{p = 1}^{4} {D}_{p}}{{\sum }_{p = 2}^{4} {Eu}_{p}} ) \qquad ( 15 )$$

Dₚ—Shannon Diversity of passage “p”
Euₚ—Euclidean distance between composition vectors of passages p and p−1

### REFERENCES

1. Giles, E. M., D'Adamo, G. L. & Forster, S. C. The future of faecal transplants. Nat. Rev. Microbiol. 17, 719 (2019).
2. Lamousé-Smith, E., Kelly, D. & De Cremoux, I. Designing bugs as drugs: exploiting the gut microbiome. Am. J. Physiol. Liver Physiol. (2020) doi:10.1152/ajpgi.00381.2019.
3. DeFilipp, Z. et al. Drug-Resistant E. coli Bacteremia Transmitted by Fecal Microbiota Transplant. N. Engl. J. Med. 381, 2043-2050 (2019).
4. Sheth, R. U., Cabral, V., Chen, S. P. & Wang, H. H. Manipulating Bacterial Communities by in situ Microbiome Engineering. Trends Genet. 32, 189-200 (2016).
5. Malone, L., Cm, M. B. A., Grigorenko, E. & Stalons, D. Weight Gain After Fecal Microbiota Transplantation. 2, 2633851 (2017).
6. Merrick, B. et al. Regulation, risk and safety of Faecal Microbiota Transplant. Infect. Prev. Pract. 2, 100069 (2020).
7. Olle, B. Medicines from microbiota. Nat. Biotechnol. 31, 309-315 (2013).
8. Ainsworth, C. Engineering the microbiome. Nature 577, 520-22 (2020).
9. Fischbach, M. A., Bluestone, J. A. & Lim, W. A. Cell-based therapeutics: The next pillar of medicine. Sci. Transl. Med. 5, 1-7 (2013).
10. Tanoue, T. et al. A defined commensal consortium elicits CD8 T cells and anti-cancer immunity. Nature 565, 600-605 (2019).
11. Petrof, E. O. et al. Stool substitute transplant therapy for the eradication of Clostridium difficile infection: ‘RePOOPulating’ the gut. Microbiome 1, 1 (2013).
12. Denault, J. Standardization and Opportunities in Manufacturing Microbiome Therapeutics.
13. Toole, P. W. O., Marchesi, J. R. & Hill, C. Next-generation probiotics: the spectrum from probiotics to live biotherapeutics. Nat. Publ. Gr. 2, 1-6 (2017).
14. Rinninella, E. et al. What is the healthy gut microbiota composition?A changing ecosystem across age, environment, diet, and diseases. Microorganisms 7, (2019).
15. Suez, J. et al. Post-Antibiotic Gut Mucosal Microbiome Reconstitution Is Impaired by Probiotics and Improved by Autologous FMT. Cell 174, 1406-1423.e16 (2018).
16. Blanton, L. V., Barratt, M. J., Charbonneau, M. R., Ahmed, T. & Gordon, J. I. Childhood undernutrition, the gut microbiota, and microbiota-directed therapeutics. Science (80-.). 352, (2016).
17. Bill and Melinda Gates Foundation. Microbial Biotherapeutics | Global Grand Challenges. Online Article https://gcgh.grandchallenges.org/challenge/new-approaches-manufacturing-gut-microbial-biotherapeutics-round-22 (2018).
18. Kumar, M., Ji, B., Zengler, K. & Nielsen, J. Modelling approaches for studying the microbiome. Nat. Microbiol. 4, 1253-1267 (2019).
19. Gilman, J., Walls, L., Bandiera, L. & Menolascina, F. Statistical Design of Experiments for Synthetic Biology. ACS Synth. Biol. 10, 1-18 (2021).
20. Cambray, G., Guimaraes, J. C. & Arkin, A. P. Evaluation of 244,000 synthetic sequences reveals design principles to optimize translation in Escherichia coli. Nat. Biotechnol. 36, 1005 (2018).
21. Xu, P., Rizzoni, E. A., Sul, S. Y. & Stephanopoulos, G. Improving metabolic pathway efficiency by statistical model-based multivariate regulatory metabolic engineering. ACS Synth. Biol. 6, 148-158 (2017).
22. Singleton, C. et al. A design of experiments approach for the rapid formulation of a chemically defined medium for metabolic profiling of industrially important microbes. PLoS One 14, 7−11 (2019).
23. Azubuike, C. C., Edwards, M. G., Gatehouse, A. M. R. & Howard, T. P. Applying statistical design of experiments to understanding the effect of growth medium components on cupriavidus necator H16 Growth. Appl. Environ. Microbiol. 86, (2020).
24. Clark, R. L. et al. Design of synthetic human gut microbiome assembly and butyrate production. Nat. Commun. 12, 1-16 (2021).
25. Medlock, G. L. et al. Inferring Metabolic Mechanisms of Interaction within a Defined Gut Microbiota. Cell Syst. 7, 245-257 (2018).
26. Faith, J. J., McNulty, N. P., Rey, F. E. & Gordon, J. I. Predicting a human gut microbiota's response to diet in gnotobiotic mice. Science (80-.). 333, 101-104 (2011).
27. Venturelli, O. S. et al. Deciphering microbial interactions in synthetic human gut microbiome communities. Mol. Syst. Biol. 14, e8157 (2018).
28. De Vos, M. G. J., Zagorski, M., McNally, A. & Bollenbach, T. Interaction networks, ecological stability, and collective antibiotic tolerance in polymicrobial infections. Proc. Natl. Acad. Sci. U.S.A 114, 10666-10671 (2017).
29. Zaramela, L. S. et al. The sum is greater than the parts: exploiting microbial communities to achieve complex functions. Curr. Opin. Biotechnol. 67, 149-157 (2021).
30. Wagner, B. D. et al. On the use of diversity measures in longitudinal sequencing studies of microbial communities. Front. Microbiol. 9, (2018).
31. Raman, A. S. et al. A sparse covarying unit that describes healthy and impaired human gut microbiota development. Science 365, (2019).
32. Lopez-siles, M., Duncan, S. H., Garcia-gil, L. J. & Martinez-medina, M. Faecalibacterium prausnitzii: from microbiology to diagnostics and prognostics. 841-852 (2017) doi:10.1038/ismej.2016.176.
33. Fischbach, M. A. & Sonnenburg, J. L. Review Eating For Two: How Metabolism Establishes Interspecies Interactions in the Gut. CHOM10, 336-347 (2011).
34. Thauer, R. K., Jungermann, K., Decker, K. & Pi, P. P. H. —Energy Conservation in Chemotrophic Anaerobic Bacteria. 41, 100-180 (1977).
35. Carmody, R. N. et al. Article Diet Dominates Host Genotype in Shaping the Murine Gut Microbiota. Cell Host Microbe 17, 72-84 (2015).
36. Smith, E. A. & Macfarlane, G. T. Dissimilatory amino acid metabolism in human colonic bacteria. Anaerobe 3, 327-337 (1997).
37. Cremer, J., Arnoldini, M. & Hwa, T. Effect of water flow and chemical environment on microbiota growth and composition in the human colon. Proc. Natl. Acad. Sci. U.S.A. 114, 6438-6443 (2017).
38. D'hoe, K. et al. Integrated culturing, modeling and transcriptomics uncovers complex interactions and emergent behavior in a three-species synthetic gut community. Elife 7, 1-29 (2018).
39. Goos, P. & Jones, B. Optimal Design of Experiments: A Case Study Approach. (John Wiley & Sons, Ltd, 2011).
40. Jones, B. & Nachtsheim, C. J. A class of three-level designs for definitive screening in the presence of second-order effects. J. Qual. Technol. 43, 1-15 (2011).
41. Rossi, O. et al. Faecalibacterium prausnitzii A2-165 has a high capacity to induce IL-10 in human and murine dendritic cells and modulates T cell responses. Nat. Publ. Gr. 1−12 (2015) doi:10.1038/srep18507.
42. Louis, P. & Flint, H. J. Diversity, metabolism and microbial ecology of butyrate-producing bacteria from the human large intestine. FEMS Microbiol. Lett. 294, 1-8 (2009).
43. Microbe, H. G. Functional Metabolic Map of Faecalibacterium prausnitzii, a Beneficial Human Gut Microbe. 3289-3302 (2014) doi:10.1128/JB.01780-14.
44. Venturelli, O. S. et al. Deciphering microbial interactions in synthetic human gut microbiome communities. 1-35 (2017).
45. Goldford, J. E. et al. Emergent simplicity in microbial community assembly. Science (80-.). 361, 469-474 (2018).
46. Peng, X. “Nick”, Gilmore, S. P. & O'Malley, M. A. Microbial communities for bioprocessing: lessons learned from nature. Curr. Opin. Chem. Eng. 14, 103-109 (2016).
47. Zhang, H. & Wang, X. Modular co-culture engineering, a new approach for metabolic engineering. Metab. Eng. 37, 114-121 (2016).
48. Roell, G. W. et al. Engineering microbial consortia by division of labor. Microb. Cell Fact. 18, 1-11 (2019).
49. Zhou, K., Qiao, K., Edgar, S. & Stephanopoulos, G. Distributing a metabolic pathway among a microbial consortium enhances production of natural products. Nat. Biotechnol. 33, 377-383 (2015).
50. MacArthur, R. Species packing and competitive equilibrium for many species. Theor. Popul. Biol. 1, 1-11 (1970).
51. Coyte, K. Z. & Schluter, J. The ecology of the microbiome: Networks, competition, and stability. 350, (2015).
52. Gonze, D., Coyte, K. Z., Lahti, L. & Faust, K. ScienceDirect Microbial communities as dynamical systems. Curr. Opin. Microbiol. 44, 41-49 (2018).
53. Lawson, C. E. et al. Common principles and best practices for engineering microbiomes. Nat. Rev. Microbiol. 17, 725-741 (2019).
54. David, L. A. et al. Diet Rapidly and Reproducibly Alters the Human Gut Microbiome. Nature 505, 559-563 (2014).
55. Garber, K. First microbiome-based drug clears phase III, in clinical trial turnaround. Nat. Rev. Drug Discov. 19, 655-656 (2020).
56. Hromada, S. et al. Negative interactions determine Clostridioides difficile growth in synthetic human gut communities. Mol. Syst. Biol. 17, (2021).
57. Zhang, J., Kobert, K., Flouri, T. & Stamatakis, A. PEAR: A fast and accurate Illumina Paired-End reAd mergeR. Bioinformatics 30, 614-620 (2014).
58. Wang, Q., Garrity, G. M., Tiedje, J. M. & Cole, J. R. Naïve Bayesian classifier for rapid assignment of rRNA sequences into the new bacterial taxonomy. Appl. Environ. Microbiol. 73, 5261-5267 (2007).
59. Schloss, P. D. et al. Introducing mothur: Open-source, platform-independent, community-supported software for describing and comparing microbial communities. Appl. Environ. Microbiol. 75, 7537-7541 (2009).
60. Cremer, J., Arnoldini, M. & Hwa, T. Effect of water flow and chemical environment on microbiota growth and composition in the human colon. 2-7 (2017) doi:10.1073/pnas.1619598114.

1. A method of designing production of a microbial community comprising different microbe subsets of a set of microbes in a proportion approximating a target proportion and, optionally, producing the microbial community, the method comprising:
(1) designing a target medium, comprising:
(1a) growing each of the microbe subsets separately to carrying capacity in each of multiple test media comprising different test-concentration profiles;
(1b) determining proportion of microbe subset carrying capacities in each test medium to thereby obtain test-medium proportions corresponding to the test-concentration profiles; and
(1c) determining from the test-medium proportions and the corresponding test-concentration profiles a target-concentration profile yielding or predicting to yield a target-medium proportion of microbe subset carrying capacities that differs from at least one of the test-medium proportions, wherein the target medium is defined as comprising the target-concentration profile; and/or
(2) designing a target-inoculum-density profile, comprising:
(2a) growing to stationary phase the microbe subsets together in each of multiple inoculum test cultures inoculated with different test-inoculum-density profiles of the microbe subsets;
(2b) determining proportion of the microbe subsets at stationary phase in each inoculum test culture to thereby obtain test-inoculum-density proportions corresponding to the test-inoculum-density profiles; and
(2c) determining the target-inoculum-density profile from the test-inoculum-density proportions and the corresponding test-inoculum-density profiles, wherein the target-inoculum-density profile yields or is predicted to yield a target-inoculum-density proportion of the microbe subsets that differs from at least one of the test-inoculum-density proportions.

2. (canceled)

3. The method of claim 1, wherein the target-medium proportion is a proportion more similar to the target proportion than each of the test-medium proportions.

4. The method of claim 1, wherein step (1c) comprises modeling the test-medium proportions and the corresponding test-concentration profiles to obtain predicted-carrying-capacity proportions of the microbe subsets and corresponding predicted-concentration profiles, wherein the target-concentration profile is one of the predicted-concentration profiles corresponding to one of the predicted-carrying-capacity proportions with maximized similarity to the target proportion.

5-6. (canceled)

7. The method of claim 1, wherein the test-concentration profiles comprise different concentrations of one or more of a carbohydrate, an amino acid, a polypeptide, a lipid, a salt, and pH.

8. (canceled)

9. The method of claim 1, wherein the target-inoculum-density proportion is a proportion more similar to the target proportion than each of the test-inoculum-density proportions.

10. The method of claim 1, wherein step (2c) comprises modeling the test-inoculum-density proportions and the corresponding test-inoculum-density profiles to obtain predicted-steady-state proportions of the microbe subsets and corresponding predicted-inoculum-density profiles, wherein the target-inoculum-density profile is one of the predicted-inoculum-density profiles corresponding to one of the predicted-steady-state proportions with maximized similarity to the target proportion.

11-12. (canceled)

13. The method of claim 1, wherein steps (2a)-(2c) of step (2) are performed iteratively.

14. The method of claim 13, wherein the iterative performance of steps (2a)-(2c) comprises a prior performance of step (2c) and a subsequent performance of step (2a), and wherein step (2) further comprises, between the prior performance of step (2c) and the subsequent performance of step (2a), (2d) determining the test-inoculum-density profiles of the subsequent performance of step (2a) as points in a multivariate design space encompassing the target-inoculum-density profile in the prior performance of step (2c).

15. The method of claim 1, wherein step (2) further comprises:
(3) designing initial-test-inoculum-density profiles, wherein the initial-test-inoculum-density profiles comprise the test-inoculum-density profiles in a first performance of step (2a), wherein the designing the initial-test-inoculum-density profiles comprises:
(3a) growing each of the microbe subsets separately in a set of first inoculum test cultures and measuring kinetic growth of each microbe subset in its first inoculum test culture;
(3b) growing each of the microbe subsets together in a second inoculum test culture and measuring total growth limit of the microbe subsets in the second inoculum test culture;
(3c) modeling the kinetic growth of each microbe subset and the total growth limit to solve for a reference-inoculum-density profile; and
(3d) determining the initial-test-inoculum-density profiles as points in a multivariate design space encompassing the reference-inoculum-density profile.

16. The method of claim 15, wherein the reference-inoculum-density profile is predicted to yield an initial-target-inoculum-density proportion of microbe subsets at steady state, and wherein the initial-target-inoculum-density proportion is a proportion predicted in the modeling of step (3c) as having maximized similarity to the target proportion.

17. (canceled)

18. The method of claim 15, wherein the growing of steps (3a) and (3b) is performed in the target medium.

19. The method of claim 1, wherein each microbe subset comprises no more than one microbial genus.

20. The method of claim 1, wherein each microbe subset comprises no more than one microbial species.

21. The method of claim 1, wherein the set of microbes comprises from 2 to 50 microbe subsets.

22-23. (canceled)

24. The method of claim 1, wherein the method comprises steps (1) and step (2).

25. The method of claim 24, wherein the growing of step (2a) is performed in the target medium.

26. The method of claim 1, wherein the target proportion is an equal relative abundance of each microbe subset.

27. The method of claim 1, further comprising:
producing the microbial community, comprising inoculating a microbial community medium with the microbe subsets, wherein:
the microbial community medium is the target medium; and/or
the microbial community medium is inoculated with the microbe subsets in the target-inoculum-density profile; and
growing the microbe subsets.

28. (canceled)

29. A microbial community produced by a method as recited in claim 1.
